SEQUENCE LISTING (146 sequences)

SEQ ID NO: 1 - protein, 832 aa, Thermus aquaticus [sequence omitted]

SEQ ID NO: 2 - protein, 832 aa, synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLTLIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPRVRAWIEKTLEEGRRRGYVETLFGRRRYVPDLEARVKSVRKAAERKAFNMPVQGTAADLMRLAMVKLFPRLEEMGARMLLQVHDELVLEAPKERAEAVARLAKEVMEGVYPLAVPLEVEVGIGEDWLSAKE

SEQ ID NO: 3 - protein, 832 aa, synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLTLIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPRVRAWIEKTLEEGRRRGYVETLFGRRRYVPNLEARVKSVREAAERMAFNMPVQGTAADLMRLAMVKLFPRLEEMGARMLLQVHDELVLEAPKERAEAVARLAKEVMEGVYPLAVPLEVEVGIGEDWLSAKE

SEQ ID NO: 4 - protein, 832 aa, synthetic construct
MRGMLPLFEPKGRVLLVDGHHLPYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYPITPAWLWEKYGLRPDQWADYRALTGDESDNLPGVKGIGEKTARKLLEEWGSLEALLKNLDRLKPAVREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSREVAEEIARLEAEVFRLAGHPFNLNSRDQLERVLFDELGLPAIGKTEKTGKRSTSAAVLEVLREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLGARVKSVRKAAERKAINMPVQGTAADLMKLAMVKMFPRLEEMGARMLLQVHDELVLEAPKERAEAVARLAKEVMEGVYPLAVPLEVEVGIGEDWLSAKE

SEQ ID NO: 5 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPNLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRKAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRRAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 8 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRNAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 9 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRKAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 10 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRKAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 11 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRRAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 12 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRRAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 13 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRNAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 14 - protein, 832 aa, synthetic construct
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRNAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 15 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPNLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 16 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRKAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 17 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRRAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 18 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRNAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 19 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRKAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 20 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRKAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 21 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRRAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 22 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRRAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 23 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRNAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 24 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVRMAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 25 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPNLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 26 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRKAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 27 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRRAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 28 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRNAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 29 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRKAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 30 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRKAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 31 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLREGRRRGYVETLFGRRRYVPDLEARVKSVREAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 32 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRRAAERNAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 33 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRNAAERKAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 34 - protein, 832 aa, synthetic construct
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVRNAAERRAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 35 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPNLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 36 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERKAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 37 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERRAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 38 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERNAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 39 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERRAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 40 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERNAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 41 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERKAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 42 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERNAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 43 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERKAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 44 - skipped

SEQ ID NO: 45 - protein, 832 aa, synthetic construct
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 46 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPNLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 47 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERKAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 48 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERRAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 49 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERNAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 50 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERRAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 51 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERNAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 52 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERKAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 53 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERNAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 54 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERKAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 55 - protein, 832 aa, synthetic construct
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 56 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPNLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 57 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERKAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 58 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERRAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 59 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERNAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 60 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERRAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 61 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERNAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 62 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERKAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 63 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERNAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 64 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERKAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 65 - protein, 832 aa, synthetic construct
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 66 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPNLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 67 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERKAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 68 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERRAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 69 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERNAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 70 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERRAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 71 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERNAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 72 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERKAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 73 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERNAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 74 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERKAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 75 - protein, 832 aa, synthetic construct
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKN

SEQ ID NO: 76 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPNLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 77 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERKAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 78 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERRAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 79 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERNAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 80 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERRAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 81 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERNAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 82 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERKAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 83 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRRAAERNAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 84 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERKAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 85 - protein, 832 aa, synthetic construct
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRNAAERRAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 86 - DNA, 40 bp, synthetic construct
  source 1..40: mol_type = other DNA
caggaaacagctatgacaaaaatctagataacgagggcaa

SEQ ID NO: 87 - DNA, 45 bp, synthetic construct
  source 1..45: mol_type = other DNA
gtaaaacgacggccagtagcttagttagatatcagagaccatggt

SEQ ID NO: 88 - DNA, 17 bp, synthetic construct
  source 1..17: mol_type = other DNA
caggaaacagctatgac

SEQ ID NO: 89 - DNA, 17 bp, synthetic construct
  source 1..17: mol_type = other DNA
gtaaaacgacggccagt

SEQ ID NO: 90 - DNA, 41 bp, synthetic construct
  source 1..41: mol_type = other DNA
gaatagttcgacaaaaatctagataacgagggcaaaaaatg

SEQ ID NO: 91 - DNA, 37 bp, synthetic construct
  source 1..37: mol_type = other DNA
cctgcaggtcgacttattctttcgcgctcagccagtc

SEQ ID NO: 92 - DNA, 21 bp, synthetic construct
  source 1..21: mol_type = other DNA
atgactgcaaagatggaaacg

SEQ ID NO: 93 - DNA, 23 bp, synthetic construct
  source 1..23: mol_type = other DNA
tcaaaatgtttgcaactgctgcg

SEQ ID NO: 94 - DNA, 18 bp, synthetic construct
  source 1..18: mol_type = other DNA
tgttctggctgtgcagtt

SEQ ID NO: 95 - DNA, 27 bp, synthetic construct
  source 1..27: mol_type = other DNA
caccaccaccgtggtatgctgccgctg

SEQ ID NO: 96 - DNA, 41 bp, synthetic construct
  source 1..41: mol_type = other DNA
atgatgatgcattttttgccctcgttatctagatttttgtc

SEQ ID NO: 97 - DNA, 52 bp, synthetic construct
  source 1..52: mol_type = other DNA
tcgtggtggtgatgatgatgcattttttgccctcgttatctagatttttgtc

SEQ ID NO: 98 - DNA, 37 bp, synthetic construct
  source 1..37: mol_type = other DNA
gaacctgtacttccagtcccgtggtatgctgccgctg

SEQ ID NO: 99 - DNA, 23 bp, synthetic construct
  source 1..23: mol_type = other DNA
ggtcacccgttcaacctgaacag

SEQ ID NO: 100 - DNA, 22 bp, synthetic construct
  source 1..22: mol_type = other DNA
gtcaaccgccttcacgcggaac

SEQ ID NO: 101 - DNA, 34 bp, synthetic construct
  source 1..34: mol_type = other DNA
  modified_base 1: mod_base = OTHER; note = 5' fluorescein tag
gttttcccagtcacgacgttgtaaaacgacggcc

SEQ ID NO: 102 - DNA, 24 bp, synthetic construct
  source 1..24: mol_type = other DNA
aaatctagataacgagggcaaaaa

SEQ ID NO: 103 - DNA, 18 bp, synthetic construct
  source 1..18: mol_type = other DNA
gtctgcggtcagaatacg

SEQ ID NO: 104 - DNA, 21 bp, synthetic construct
  source 1..21: mol_type = other DNA
gagaaagaaggttacgaggtt

SEQ ID NO: 105 - DNA, 18 bp, synthetic construct
  source 1..18: mol_type = other DNA
accgaactccagacgttc

SEQ ID NO: 106 - DNA, 15 bp, synthetic construct
  source 1..15: mol_type = other DNA
ctgcgtgcgttcctg

SEQ ID NO: 107 - DNA, 15 bp, synthetic construct
  source 1..15: mol_type = other DNA
accccacaggttcgc

SEQ ID NO: 108 - DNA, 18 bp, synthetic construct
  source 1..18: mol_type = other DNA
ctgagcgaacgtctgttc

SEQ ID NO: 109 - DNA, 18 bp, synthetic construct
  source 1..18: mol_type = other DNA
ggtacgcgggtgaatcag

SEQ ID NO: 110 - DNA, 15 bp, synthetic construct
  source 1..15: mol_type = other DNA
gacccgctgccggac

SEQ ID NO: 111 - DNA, 21 bp, synthetic construct
  source 1..21: mol_type = other DNA
gtaacgttcgatgaacgcttg

SEQ ID NO: 112 - DNA, 18 bp, synthetic construct
  source 1..18: mol_type = other DNA
gcgattccgtacgaggaa

SEQ ID NO: 113 - DNA, 15 bp, synthetic construct
  source 1..15: mol_type = other DNA
cccctgcaggtcgac

SEQ ID NO: 114 - DNA, 59 bp, synthetic construct
  source 1..59: mol_type = other DNA
tagcgaaggatgtgaacctaatccctgctcccgcggccgatctgccggccgcgggagca

SEQ ID NO: 115 - DNA, 20 bp, synthetic construct
  source 1..20: mol_type = other DNA
gacgcctacgaaaacctcac

SEQ ID NO: 116 - DNA, 20 bp, synthetic construct
  source 1..20: mol_type = other DNA
ccgagaagaagccccagtag

SEQ ID NO: 117 - DNA, 20 bp, synthetic construct
  source 1..20: mol_type = other DNA
gcgacgcctacgaaaacctc

SEQ ID NO: 118 - DNA, 18 bp, synthetic construct
  source 1..18: mol_type = other DNA
gacgcagcgaccacaagc

SEQ ID NO: 119 - DNA, 19 bp, synthetic construct
  source 1..19: mol_type = other DNA
tctggccaccatgcgaagc

SEQ ID NO: 120 - DNA, 21 bp, synthetic construct
  source 1..21: mol_type = other DNA
accagttgagcaggtactggg

SEQ ID NO: 121 - DNA, 18 bp, synthetic construct
  source 1..18: mol_type = other DNA
ccgcagcacatccacgat

SEQ ID NO: 122 - DNA, 19 bp, synthetic construct
  source 1..19: mol_type = other DNA
cgcagcggcatgtcctgct

SEQ ID NO: 123 - DNA, 18 bp, synthetic construct
  source 1..18: mol_type = other DNA
atccccgagtgcagctgg

SEQ ID NO: 124 - DNA, 21 bp, synthetic construct
  source 1..21: mol_type = other DNA
gtattaaaaggtactggtgga

SEQ ID NO: 125 - DNA, 23 bp, synthetic construct
  source 1..23: mol_type = other DNA
ctattgttggatcatattcgtcc

SEQ ID NO: 126 - DNA, 17 bp, synthetic construct
  source 1..17: mol_type = other DNA
cgtccccacctgcgtca

SEQ ID NO: 127 - DNA, 18 bp, synthetic construct
  source 1..18: mol_type = other DNA
cagcgctcacggggtagg

SEQ ID NO: 128 - DNA, 18 bp, synthetic construct
  source 1..18: mol_type = other DNA
tgcgggccaagccgggga

SEQ ID NO: 129 - DNA, 24 bp, synthetic construct
  source 1..24: mol_type = other DNA
ggtaggagtaggcgccgatgtcct

SEQ ID NO: 130 - DNA, 17 bp, synthetic construct
  source 1..17: mol_type = other DNA
tcgtggagcccgacaag

SEQ ID NO: 131 - DNA, 19 bp, synthetic construct
  source 1..19: mol_type = other DNA
ggcagatggggacacaggt

SEQ ID NO: 132 - DNA, 24 bp, synthetic construct
  source 1..24: mol_type = other DNA
gcttatctactccatcgccttcac

SEQ ID NO: 133 - DNA, 19 bp, synthetic construct
  source 1..19: mol_type = other DNA
gccacagccagggtgttga

SEQ ID NO: 134 - DNA, 22 bp, synthetic construct
  source 1..22: mol_type = other DNA
gctggtatcgaacaagctgaag

SEQ ID NO: 135 - DNA, 18 bp, synthetic construct
  source 1..18: mol_type = other DNA
gacacctcgcagcggacc

SEQ ID NO: 136 - DNA, 18 bp, synthetic construct
  source 1..18: mol_type = other DNA
ccccaaagtggacaaccg

SEQ ID NO: 137 - DNA, 17 bp, synthetic construct
  source 1..17: mol_type = other DNA
gctggagcgggcgttct

SEQ ID NO: 138 - DNA, 17 bp, synthetic construct
  source 1..17: mol_type = other DNA
gcaggctcggaggtggg

SEQ ID NO: 139 - DNA, 18 bp, synthetic construct
  source 1..18: mol_type = other DNA
gtcgatgcggggagttcg

SEQ ID NO: 140 - DNA, 18 bp, synthetic construct
  source 1..18: mol_type = other DNA
agtgcatggaggcggacc

SEQ ID NO: 141 - DNA, 20 bp, synthetic construct
  source 1..20: mol_type = other DNA
gccaagaagccccttgtgag

SEQ ID NO: 142 - DNA, 21 bp, synthetic construct
  source 1..21: mol_type = other DNA
cgtcctctgtcaagatggtcc

SEQ ID NO: 143 - DNA, 20 bp, synthetic construct
  source 1..20: mol_type = other DNA
ttgaactgcttggcgaactg

SEQ ID NO: 144 - DNA, 18 bp, synthetic construct
  source 1..18: mol_type = other DNA
gtcgtcgtgggagtcgga

SEQ ID NO: 145 - DNA, 19 bp, synthetic construct
  source 1..19: mol_type = other DNA
cgctggaggcagagctgaa

SEQ ID NO: 146 - RNA, 100 nt, synthetic construct
  source 1..100: mol_type = other RNA
cctgcgggccaagccggggaccgcccggctccccggggaggacatgaggggccagtggcgtcccctgagcgtggaggacatcggcgcctactcctacccc

(19) United States
(12) Patent Application Publication
(10) Pub. No.: US 2026/0297666 A1
(43) Pub. Date: Oct. 1, 2026

(54) COMPOSITIONS AND METHODS FOR UPREGULATION OF REVERSE TRANSCRIPTION AND REDUCTION OF SEQUENCE BIAS IN RNA SEQUENCING

(71) Applicant: 5PRIME BIOSCIENCES, INC., Mt. Laurel, NJ (US)

(72) Inventors: Raj Chakrabarti, Moorestown, NJ (US); Anisha Ghosh, Mt. Laurel, NJ (US); Xiangying Guan, Mt. Laurel, NJ (US); Gauthier Errasti, Vert le Petit (FR); Thomas Delacroix, Vert le Petit (FR)

(21) Appl. No.: 19/474,961

(22) PCT Filed: Apr. 15, 2024
(86) PCT No.: PCT/US24/24649

§ 371 Date: Oct. 13, 2025

Related U.S. Application Data
(60) Provisional application No. 63/459,012, filed on Apr. 13, 2023.

(51) Int. Cl. C12Q 1/6869; C12Q 1/48; C12Q 1/6813; C12Q 1/6848; C12Q 1/686; C12Q 1/6876

(52) U.S. Cl. CPC C12Q 1/6869; C12Q 1/48; C12Q 1/6813; C12Q 1/6848; C12Q 1/686; C12Q 1/6876; C12Y 207/07007; C12Y 207/07049

(57) ABSTRACT

Compositions and methods for the upregulation of reverse transcription (RT) reactions and the reduction of sequence bias in RNA sequencing are presented. The compositions and methods include the application of certain organic cosolvents to increase the enzymatic activity of RT enzymes above their activity in aqueous media in addition to reducing the secondary structure of RNA, and/or the introduction of amino acid mutations that render the enzymes even more active in the presence of the organic cosolvents.

### BACKGROUND OF THE INVENTION

### Enzymes in Organic Solvents & Aqueous-Organic Media

Enzymes have evolved in nature for catalyzing reactions in water. Use of media that deviate from water and enter into the domain of organic solvents is a human invention to fit special needs of in vitro application of enzymes for research, clinical and industrial applications. Seen from this angle, the study of enzymatic reactions in aqueous-organic media is a field of inquiry unto itself.

Enzymatic reactions in the presence of organic solvents can span an entire spectrum ranging from mostly organic to mostly aqueous. One can also include reactions in biphasic mixtures composed of water and water-immiscible organic solvents where the former is held in suspension in the latter or vice versa (Koskinen and Klibanov, 1996). When it comes to organic media, some amount of water, even if it is in trace concentration, is always necessary for enzymes to work. As Kuntz and Kauzmann put it, water is “enzymes' molecular lubricant” (Kuntz et al., 1974). An important observation from prior extensive studies was that the activity of enzymes was almost universally if not universally reduced in mixed aqueous-organic media. For improvements in activity or selectivity against specified substrates, an enzyme must typically be placed in neat organic media with a residual water layer surrounding the enzyme.

Most of the enzymatic reactions that have been studied so far in the presence of organic solvents are hydrolytic enzymes. Moreover, the reactions have been confined to small organic molecules as substrates as opposed to enzymes that carry out other types of reactions upon biological macromolecules. Polymerase reactions (including but not limited to transcription, reverse transcription and PCR) belong to the latter class, and as such the study of such reactions in the presence of organic solvents represents a significant deviation from the prior art on enzymatic reactions in such media. In addition, only mixed aqueous-organic media (and not neat organic media) can be compatible with polymerase-catalyzed reactions due to the requirement of water to dissolve nucleic acid substrates. Hence it is expected based on all prior art that the application of organic solvents (mixed aqueous-organic media) will reduce the catalytic activity of all polymerase enzymes.

### Polymerases in Aqueous-Organic Media

Elevated temperature and organic cosolvents represent the two primary means for achieving denaturation of macromolecules. DNA polymerases, which catalyze the polymerization of DNA based on either RNA or DNA templates, have been classified into seven families (A, B, C, D, X, Y, and RT) based on their amino acid sequences and specific activities. Thermostable polymerase enzymes, which evolved naturally, revolutionized biotechnology by enabling the denaturation of nucleic acids at elevated temperatures while maintaining polymerase enzyme structure and activity in the context of the polymerase chain reaction (PCR). Recently, thermostable reverse transcriptase (RT) enzymes have been also been discovered. However, many genes cannot be effectively replicated or amplified in water irrespective of temperature, resulting in significant biases in RNA and DNA sequencing that limit the potentially transformative applications of these methods.

In particular, (RT)-PCR amplification of GC-rich nucleotide sequences is often accompanied by inadequate yield of the target DNA sequence and amplification of nonspecific products. A recent analysis of intragenic regions reveals 773 sequences with more than 65% GC in the human genome. Due to high GC content, the amplification of the target DNA requires special (RT)-PCR protocols, organic solvent additives or addition of nucleotide analogs. Organic solvent additives, also known as “PCR-enhancing compounds”, have been used to improve GC-rich gene amplification or reduce GC bias in amplification without target modification, and are components of some of the most commonly used PCR commercial products, because organic cosolvents and temperature represent the two primary means of denaturing macromolecules. Although PCR-enhancing organic solvents are very effective in improving amplification due to their favorable effects on duplex nucleic acid melting and single-stranded nucleic acid secondary structure alleviation—thus complementing the effects of the increased temperatures used in PCR, which alone are insufficient to amplify many GC-rich genes—their application is limited by the fact that they generally deleteriously affect the polymerase stability and activity. Whereas polymerases used in PCR (and in some cases in reverse transcription) have evolved naturally to be thermostable, they have not evolved naturally to be optimally solvent-tolerant.

Despite the advent of methods like unique molecular identifiers (UMIs), the problem of sequence bias in nucleic acid sequencing remains unsolved irrespective of the polymerase enzyme used, since while base calling errors are easily error-corrected by consensus sequencing, differential amplification efficiencies can induce over 100-fold changes in sequence coverage—the effects of which cannot be eliminated by digital barcoding, especially for the most important problems of rare mutation quantification.

In our earlier work on DNA-dependent DNA polymerases in aqueous-organic media, we identified certain organic cosolvents that in admixture with water proved superior for PCR amplification of many substrates, particularly those with high GC-content. These organic cosolvents belonged specifically to four chemical classes that we defined as low molecular weight amides, sulfoxides, sulfones and polyols (particularly diols) (Chakrabarti, 2002, 2004; Chakrabarti et al., 2001 Nucleic Acids Res, 2001 Gene, 2002 Biotechniques; U.S. Pat. Nos. 6,949,368; 7,276,357 B2; and 7,772,358 B2). Earlier, DMF, DMSO and Glycerol were also reported to have some beneficial effects in PCR amplification of high GC targets (Sarker et al., 1990, Pomp et al., 1991, Henkel et al., 1997). A comprehensive list of the more useful members among these low molecular weight organic cosolvents is provided below and the chemical structures of some of them are shown in FIGS. 1A to 1D.

When chosen from low molecular weight amides the members are: formamide, N-methyl formamide, N,N-dimethyl formamide (DMF), acetamide, N-methylacetamide, N,N-dimethylacetamide, propionamide, isobutyramide, 2-pyrrolidone, N-methylpyrrolidone (NMP), N-hydroxyethyl pyrrolidone (HEP), N-formyl pyrrolidine, N-Formyl morpholine; delta-valerolactam, epsilon-caprolactam, 2-azacyclooctanone (16 compounds);

When chosen from low molecular weight sulfoxides the members are: dimethyl sulfoxide (DMSO), n-propyl sulfoxide, n-butyl sulfoxide, methyl sec-butyl sulfoxide, and tetramethylene sulfoxide (5 compounds: FIG. 1B);

When chosen from low molecular weight sulfones the members are: dimethyl sulfone, 10 diethyl sulfone, di (n-propyl) sulfone, tetramethylene sulfone (sulfolane), and 2,4-dimethylsulfolane and butadiene sulfone (sulfolene)—(6 compounds: FIG. 1C);

When chosen from low molecular weight diols the members are: 1,2-propanediol, 1,3-propanediol, 1,2-butanediol, 1,3-butanediol, 1,4-butanediol, 1,2-pentanediol, 2,4-pentanediol, 1,5-pentanediol, 1,2-cyclopentanediol, 1,2-hexanediol, 1,6-hexanediol, and 2-methyl-2,4-pentanediol (13 compounds; FIG. 1D). Other than the diols a triol, namely, glycerol, as mentioned has also been found to help in enhancing amplification of certain high-GC targets. Among the other organic compounds that can belong to the preferred organic component is betaine.

When used as a part of the PCR buffer, these cosolvents provide an aqueous-organic reaction medium that is predominantly aqueous in nature (as opposed to the common use of predominantly organic reaction media for small molecule enzymatic reactions described earlier). They have been found to be especially effective in amplifying high-GC containing polynucleotide targets by providing the following benefits: a) Lowering the melting temperature of double stranded DNA: This meant better and more complete denaturation of even the very high melting DNA targets at temperatures that do not cause DNA damage, namely at or below 95° C. Targets that could not be amplified in standard aqueous buffers could now be amplified in these modified buffers. b) Better specificity of the products especially in view of the facts that these mixed aqueous-organic buffers readily opened up secondary structures in the ssDNA strands that are primary causes of pauses in the extension reactions and as such also of nonspecific product formation.

The properties of a cosolvent in terms of its overall impact on a PCR reaction can be expressed in terms of effective range, potency, and specificity of each cosolvent that are different for different compounds (Chakrabarti R., 2004). The effective range of a cosolvent is defined as the range of concentration starting at the concentration at which amplification of a given target improved PCR yield and ending at the concentration above which amplification began to be inhibited. Put in a different way, the effective range of a cosolvent is the range of concentration outside which it does not exhibit any beneficial effect. This range was different for different compounds but also for the same compound for different targets. The potency of a cosolvent is defined as the maximum densitometric volume of the target band amplification that could be obtained for any target amplification within the effective range of that cosolvent. It is the maximum effectiveness of the cosolvent at the most effective concentration within its effective range. The specificity of a cosolvent at a particular concentration is defined as the ratio of the volume of the target band amplification to the total volume of all bands, including the undesired non-specific bands, expressed as a percent. False positives and false negatives in PCR-based disease diagnosis, for instance, are the result of poor reaction specificity. Use of cosolvent-based PCR is of significant value in this area.

There are, however, important limitations of these solvent systems that have thwarted their more widespread application. Though most of the polymerase-compatible cosolvents, as defined above, provided superior PCR amplification in terms of extent of amplification and specificity of the amplified product for DNA targets—especially those DNA targets that had high GC content and defied amplification under standard conditions—their performance in many cases was severely limited by the narrow concentration ranges within which they were effective. Further investigation revealed that these deficiencies resulted from decreased polymerase stability (lower half-lives) and polymerase activity of the enzymes in the presence of these cosolvents (Chakrabarti, 2002, 2004). Both polymerase half-lives and specific enzymatic activities (primer extension rates) decreased rapidly with increasing cosolvent concentration. For example, the thermostabilities of DNA polymerases between 92° C. and 95° C.—the range within which the denaturation step of the PCR reaction is usually carried out—were greatly lowered by addition of the most potent and most specific cosolvents.

Notwithstanding the above results on DNA-dependent DNA polymerization and PCR in aqueous-organic media, reverse transcription activity (RNA-dependent DNA polymerization) has never been demonstrated to be improved by or even compatible with the aforementioned polar organic cosolvents. In fact, in the context of reverse transcription PCR (RT-PCR), which produces DNA from RNA using an RNA-dependent DNA polymerase followed by amplification of DNA by a DNA-dependent DNA polymerase, even if the aforementioned organic cosolvents are applied in the PCR reaction, they are typically excluded from the reverse transcription reaction. Historically, reverse transcriptases (RTs) employed in biotechnology were exclusively from mesophilic organisms and were not thermostable. RTs that are not thermostable generally cannot tolerate the presence of organic cosolvents, or even if they do, their activities are greatly diminished, rendering the inclusion of organic cosolvents in such reactions deleterious. Finally, the foregoing primary reason for use of organic solvents in PCR is inapplicable to reverse transcriptase reactions, since reverse transcription does not involve double-stranded nucleic acid denaturation prior to polymerization.

### SUMMARY

Compositions and methods for the upregulation of reverse transcription (RT) reactions and the reduction of sequence bias in RNA sequencing are presented. Notwithstanding previously understood principles regarding the function of enzymes in aqueous-organic media, and specifically the function of polymerase enzymes in aqueous-organic media, it has been found that inclusion of certain polar organic cosolvents in the reaction media of RT enzymes can significantly increase the activity of RT enzymes above their activity in aqueous media, in addition to reducing the secondary structure of RNA. Additionally, as described further herein, introduction of amino acid alterations can render the reverse transcriptase enzymes even more active and/or stable in the presence of such organic cosolvents.

In one aspect, a composition for performing a reverse transcriptase reaction comprises a thermostable reverse transcriptase or a fragment thereof, a reverse transcriptase (RT) buffer, one or more template RNAs and deoxyribonucleoside triphosphates (dNTPs), and one or more of the low molecular weight polar organic solvents. As described further herein, low molecular weight polar organic solvents, in some embodiments, are selected from the group consisting of an amide, a sulfoxide, a sulfone, and a diol. Low molecular weight polar organic solvents, in some embodiments, can have a molecular weight less than or equal to 150 g/mol. Additionally, the low molecular weight polar organics can be present at a concentration ranging between 0.05 molar and 7.5 molar, in some embodiments. In some embodiments, the thermostable reverse transcriptase or a fragment thereof has an optimal reverse transcriptase (RT) above 37° C. and preferably above 48° C. As shown and described further herein, the presence of the one or more of the organic cosolvents, in some embodiments, increases RT activity (rate of nucleotide incorporation) of the thermostable reverse transcriptase or the fragment thereof. For example, the RT activity rate can be increased by at least 5%, in some embodiments.

The reverse transcriptase of compositions described herein, in some embodiments, comprises one or more amino acids alterations conferring stability and/or activity in the one or more polar organic solvents. For example, the reverse transcriptase is a modified or mutant Taq polymerase bearing at least 90% sequence similarity to wild-type Taq polymerase (SEQ ID NO: 1), wherein the amino acid modifications confer stability and/or activity in the one or more polar organic solvents.

In another aspect, a composition comprises a thermostable reverse transcriptase or a fragment thereof, a reverse transcriptase polymerase chain reaction (RT-PCR) buffer, one or more template RNAs and deoxyribonucleoside triphosphates (dNTPs), DNA-dependent DNA polymerase enzyme of a fragment thereof, and one or more low molecular weight polar organic solvents. The thermostable reverse transcriptase of the composition can be any thermostable reverse transcriptase described herein. Additionally, the one or more low molecular weight solvents can have any identity described herein.

In another aspect, a composition for performing a reverse transcriptase reaction comprises a thermostable reverse transcriptase, a reverse transcriptase (RT) buffer, one or more template RNAs and deoxyribonucleoside triphosphates (dNTPs), and one or more low molecular weight polar organic solvents, wherein the thermostable reverse transcriptase is a mutant of Taq polymerase bearing at least 90% sequence similarity to wild-type Taq polymerase of SEQ ID NO:1 and comprises one or more amino acid alterations enhancing stability and/or activity of the reverse transcriptase in the one or more low molecular weight polar organic solvents.

In another aspect, modified Taq DNA polymerases are described herein. In some embodiments, a modified Taq DNA polymerase having an amino acid sequence that is at least 90% identical to an amino acid sequence comprised of the sequence of wild-type Taq DNA polymerase of SEQ ID NO:1, wherein the amino acid sequence comprises a first set of non-natural amino acid alterations stabilizing or improving the activity of the modified Taq DNA polymerase in an aqueous-organic medium, and a second set of non-natural amino acid alterations conferring reverse transcriptase activity to the modified Taq DNA polymerase. Any non-natural amino acid alterations consistent with the objectives of the first and second sets can be employed.

In another aspect, a composition comprises a modified Taq DNA polymerase suitable for RT or RT-PCR reactions in an aqueous-organic medium, wherein the aqueous-organic medium comprises one or more low molecular weight organic solvents selected from the group consisting of an amide, a sulfoxide, a sulfone, and a diol, and wherein the amino acid sequence of the modified Taq DNA polymerase is at least 90% identical to an amino acid sequence comprised of the sequence of wild-type Taq DNA polymerase of SEQ ID NO:1 with a first set of amino acid alterations selected to confer reverse transcriptase activity, and a second set of amino acid alterations selected from the group consisting of
L30P, A54V, E434D, K206Q, S612R, V7301, and F749V of SEQ ID NO:1;
P10S, A61V, T186I, D244V, K314R, E520G, V586A, S612R, V7301, and F749V of SEQ ID NO:1;
G12T, A54V, T186I, D244V, F667Y, and F749V of SEQ ID NO:1;
P10S, A61V, F73S, T186I, R205K, K219E, M236T, A608V, S612R, and 2494AG of SEQ ID NO:1;
P10S, L30P, A61V, L365P, V586A, S612R, and E832K of SEQ ID NO:1;
P10S, A61V, D244V, S612R, and E832 of SEQ ID NO:1;
L30P and 2494AG of SEQ ID NO:1; and
A29T, G200S, D237G, and F749I of SEQ ID NO:1.
   In some embodiments, the amino acid alterations conferring RT activity include one or more of E732N, E742K, E742R, M747K, M747R, E742N, E742N, E742Q, E742Y, E742M, E742A and M747N of SEQ ID NO:1. For example, in some embodiments, the second set of amino acids above are paired with one of the E742 alterations and one of the E747 alterations of SEQ ID NO:1. Any desired pairing can be made. Non-limiting examples of such pairing are provided in Table 15 below.

In another aspect, kits are provided herein. In some embodiments, a kit comprises a composition for reverse transcription or a composition for reverse transcription-PCR described herein. In some embodiments a kit comprises a composition for target enrichment for RNA sequencing by a reverse transcription or reverse transcription-PCR composition described herein.

Methods of reverse transcribing RNA into DNA and methods of administering reverse transcription-PCR are also described herein. In some embodiments, a method of reverse transcribing RNA into DNA comprises incubating a thermostable reverse transcriptase with a reverse transcriptase buffer, a RNA template, deoxyribonucleoside triphosphates (dNTPs), DNA primers, and at least one low molecular weight organic cosolvent. The one or more low molecular weight organic cosolvents can have any identity described herein. In some embodiments, the reverse transcriptase is a modified Taq DNA polymerase bearing at least 90% sequence similarity to wild-type Taq polymerase of SEQ ID NO:1. Moreover, in some embodiments, the temperature of reverse transcription is higher than its optimal value in the absence of the polar organic cosolvent, and less than or equal to the melting temperature of the enzyme at that cosolvent concentration, minus 5° C. As detailed further herein, reaction yield of reaction efficiency can be enhanced in the presence of the at least one low molecular weight solvents. Additionally, reverse transcription and/or DNA amplification, in some embodiments, is administered for target enrichment in next-generation sequencing of RNA, including wherein the copy number of RNA sequences are determined by next-generation sequencing. In some embodiments, the RNA sequencing is carried out with incorporation of unique molecular identifiers (UMI) in the adapter sequences.

In another aspect, methods of detecting bacterial or viral pathogens without sample preparation, such as directly from clinical samples, are described herein. In some embodiments, a method comprises
a) incubating a clinical sample containing a virus or bacteria with RT-PCR reagents including a thermostable or solvostable reverse transcriptase (RT), one or more low molecular weight polar organic cosolvents, optionally a thermostable or solvostable DNA-dependent DNA polymerase enzyme, RT-PCR buffer, and primers complementary to the nucleic acid sequence to be detected, at a temperature exceeding 70° C. and preferably below 80° C., to lyse the virus or bacteria and release RNA without degrading the RNA;
b) incubating the lysed clinical sample at or near the optimal temperature for reverse transcription of the RT enzyme;
c) thermal cycling of the reaction mixture to PCR amplify the resulting complementary DNA (cDNA); and
d) quantifying of the PCR product.
   In some embodiments, quantifying the PCR product can be administered by qPCR. The virus of the clinical sample, for example, can be SARS-CoV virus or other respiratory virus.

In another aspect, kits for detection via RT-PCR of viral or bacterial pathogen RNA directly from clinical samples without sample preparation are provided. In some embodiments, such a kit comprises a thermostable or solvostable reverse transcriptase (RT) enzyme, one or more of the low molecular weight polar organic cosolvents, optionally a thermostable or solvostable DNA-dependent DNA polymerase enzyme, RT-PCR buffer, and primers complementary to the nucleic acid sequence to be detected. Components of the kit, including the solvostable reverse transcriptase (RT) enzyme and low molecular weight organic solvent can have any composition and/or properties described herein.

In another aspect, methods for droplet digital reverse transcription PCR (ddRT-PCR) with enhanced RT and PCR efficiency are described herein. In some embodiments, a method for droplet digital reverse transcription PCR (ddRT-PCR) with enhanced RT and PCR efficiency, the method comprising:
a) incubating RT-PCR reagents including a RT-PCR buffer, a thermostable or solvostable reverse transcriptase, a thermostable or solvostable DNA-dependent DNA polymerase, primers and/or probes for a sequence or mutation of interest, a polar organic cosolvent within emulsion droplets, and one or more RNA templates, at or near the optimal temperature for reverse transcription, such that the polar organic cosolvent at least doubles the reverse transcription yield compared to buffer lacking the cosolvent;
b) thermal cycling of the reaction mixture to PCR amplify the resulting cDNA; and
c) counting or sorting of the resulting positive droplets using a fluorescence-based counting or sorting device.

In another aspect, methods for accelerating the in vitro evolution of reverse transcriptase (RT) enzymes are provided. In some embodiments, A method for accelerating the in vitro evolution of reverse transcriptase (RT) enzymes, the method comprises:
a) preparation of a library of thermostable polymerase enzyme gene variants;
b) expression of the enzymes corresponding to these gene variants, for example through bacterial transformation and expression or in vitro transcription/translation of the library, in individual containers, the containers preferably being either droplets or microplate wells;
c) incubation of the library of enzyme variants within individual containers, with one or more polar organic cosolvents at specified concentrations, RT-PCR buffer, primers and/or probes for an RNA sequence of interest, optionally a thermostable or solvostable DNA-dependent DNA polymerase enzyme, and the RNA template, at a temperature suitable for reverse transcription, the temperature preferably being between 48° C. and 80° C.;
d) thermal cycling of the reaction mixtures to PCR amplify the resulting cDNAs;
e) ranking of the resulting enzyme variants based on RT-PCR yield, either by screening or sorting, the screening or sorting preferably being done based on fluorescence; and
f) sequencing of the resulting top enzyme variants to identify the best RT enzymes, wherein the presence of organic cosolvent(s) at the specified concentration(s) increase the RT activity of at least one enzyme variant at least twofold above the activity in their absence.
   In some embodiments, at least one of the enzyme variants is a rare variant whose corresponding gene is present in the library with less than or equal to 1% frequency.

These and other embodiments are further described in the following detailed description.

### BRIEF DESCRIPTION OF THE DRAWINGS

FIGS. 1A-1D provide various low molecular weight solvents for use in compositions and methods described herein.

FIGS. 2A-2D provide effects of organic solvents on reverse transcriptase (RT) activities of polymerases. RT activity was measured using enzymes of interest (WT, SFM 4-6, SFM 4-3, N-7-3-B07-RT, L-5-2-F01-RT1, L-5-2-F01-RT2) in the presence of different concentrations of 1,4-butanediol, 2-pyrrolidone, sulfolane, TMSO. At extension temperature (55° C.) conducive to the reduction of RNA secondary structure (A), equal activities of each enzyme were applied, and no heat pre-treatment was applied on tested enzymes; At extension temperature (68° C.) (B), the same amount of each enzyme as in (A) was applied, and no heat pre-treatment was applied on tested enzymes; (C) The enzymes were pre-heated at 95° C. for 2 min prior RT activity assay at 68° C.; (D) At extension temperature (76° C.) no heat pre-treatment was applied on tested enzymes. Normalized fluorescence was applied for (A) and (B) and relative AFU in (C, D).

FIGS. 3A-3B address reverse transcriptase activity measurement under low template concentration and on GC-rich templates. FIG. 3(A)—Comparison of RT activity for commercial RT enzyme (50 U [62.5 ng] of ProtoScript II RT) with mutants of interest (including 25 ng of WT, N-7-3-B07-RT, L-5-2-F01-RT1, L-5-2-F01-RT2) under extension temperatures 55° C. and 68° C. The Poly(A) template concentration is 0.5 ng/ul (2.82 nM). FIG. 3(B)—The contribution of 5% BD to fluorescence readout was also assessed. The addition of 5% BD does not change the RFU. The green curve corresponds to addition of 5% BD after the reaction and before fluorescence readout.

FIG. 4 illustrates BEGAIN RNA fragment folding into robust secondary structure. Sequence of the 100-base synthesized.

FIGS. 5A-5L details performance of engineered polymerases in PCR and RT-qPCR of GC-rich templates. Panels A-H: amplification of GC-rich genomic DNA targets by L-5-2-F01 and WT-Taq were evaluated in presence of 7% and 10% BD, using two PCR cycling protocols (see Materials and Methods). High denaturation temperature—WT: (A) 0% BD (B) 7% BD; L-5-2-F01: (C) 0% BD (D) 10% BD. Moderate denaturation temperature—WT: (E) 0% BD (F) 7% BD; L-5-2-F01: (G) 0% BD (H) 7% BD. Expected amplicon sizes are mentioned (in base pair) in the Figure. M=1 kb DNA ladder, numbers 0.5 and 1 are in kbp. Target properties are described in Table 13. Panels I-L: Effects of BD and temperature on GC-rich 100 base BEGAIN RNA fragment RT-qPCR by N-7-3-R07-RT=N-7-3-B07+E742K+M747K and L-5-2-F01-RT1=L-5-2-F01+E742K+M747K. RT-qPCR efficiency of these two mutants were evaluated along with the efficiency of SFM4-6 and a thermostable MMLV enzyme, ProtoScript II, in absence and presence of BD (7%) at 55° C. (I) or 68° C. (J). RT-qPCR was carried out in 3 steps as described in Materials and Methods. (K,L) represent the melt peak traces of the respective qPCR products from 55° C. to 95° C. *PSII—ProtoScript II. Table 2 herein presents quantitative peak area and T_{M} results for panels K,L since Cq values can be affected by nonspecific amplification.

FIG. 6 illustrates distribution of selected mutations involved in enhancement of polymerase activity and induction of reverse transcriptase activity in the 3D structure of Taq polymerase with nucleic acid (DNA/RNA) in open conformation. As reverse transcriptase (RT) activity inducing mutations in our study were also discovered, it is suggested that RNA might bind in a similar conformation as DNA for catalytic activity. Individual subdomains of Taq polymerase ternary complex with DNA in open conformation (generated using Modeller based on PDBs—1TAU, 6Q4V, 1NK4, 3KTQ, 1BGX) are shaded magenta, grey, yellow, blue and red to highlight the 5′→3′ exonuclease, inactive 3′→5′ endonuclease, palm, thumb, and finger domains respectively. Bound DNA molecule shown as cartoon ribbon (orange). Mutations identified in five of our top performing mutants (N-7-3-C08, N-7-2-E02, N-7-3-B07, L-5-2-F01, and L-5-3-D04) and fastest enriching mutations from NGS convergence analysis are depicted. Mutations such as P10S, A23P, L30P, A54V, A61V, F73S, A97T, A118V, L162P, T186I, K206Q, I228V, D244V, K314R, L365Q, E434D, L461R, F482I, E507K, A521V, Q534R, V586A, A608V, S612R, F667Y, Q680R, K702R, E742K, V7301, E734G, F749I, F749V, K762R, L768M, and E832K are represented as cyan spheres. The RT activity inducing mutations identified in our study such as E742K, M747K are represented as purple spheres. The two images are rotated by 180 degrees to enable visualization of all mutations. Predicted effects of subset combinations of these mutations on polymerase folding free energy are reported in Table 7.

FIGS. 7A-7J: Microfluidic preparation of double emulsions and FACS sorting: We prepared primary water-in-oil emulsions FIG. 7(A) followed by PCR. A fraction of primary emulsion was used to isolate DNA and ran on 1% Agarose gel FIG. 7(B). Panel B: Lane 1 DNA marker, Lane 2 negative control, and lane 3 is positive control. Post-PCR on primary emulsions containing WT and the SPC9 engineered polymerase respectively, primary emulsions were collected to prepare double emulsions FIG. 7(C) as described in Materials and Methods section. Double emulsion (DE) is depicted in FIG. 7(D). Post-PCR, positive control was stained with SYBR Green I and visualized under a fluorescent microscope FIG. 7(E). DEs from WT and SPC9 were mixed in 90:10 proportion for sorting. The mixed double emulsion was subjected to FACS sorting. Pre-sort and post-sort images are shown in panel FIG. 7F and FIG. 7G respectively. A total 1.6 million events were randomly captured; a threshold of 5000 was applied to gate the parental DE (FIG. 7H, and FIG. 7I), followed by sorting SYBR positive double emulsion (J). Panel H depicts the total stained DEs and gating of the P1 population (black=SYBR negative DEs); FIG. 7I depicts the gated population from P1 that is identified for further analysis as P2; FIG. 7J depicts the sorted SYBR positive DEs that were collected. Orange=SYBR^{HIGH} (SPC9), purple=SYBR^{MEDIUM} (mixture of WT and SPC9), and blue=SYBR^{LOW} (WT). SSC: Side-scattered light; FSC: Forward scattered light; A: Area; H: Height.

FIGS. 7K-7L: (K) FACS sorting of the L5 library; (L) Direct fluorescence detection of reverse transcription products within emulsion droplets. 5 μg of brain total RNA was mixed with a BEGAIN gene-specific primer and processed through a dolomite microfluidic device to generate ~3 million droplets. Half of the droplets underwent RT-PCR amplification, then were stained with Picogreen and imaged under a fluorescence microscope (upper right). Pre-PCR droplets are shown for comparison (upper left). A negative control with no RNA added is also provided (lower).

FIGS. 8A-8C: FIG. 8(A) Mapping of selected mutations in top polymerase variants. FIG. 8(B) Proximity of mutated residues relevant to reverse transcriptase activity to RNA and depiction of H-bonds formed with RNA in minimized Taq polymerase ternary complex. Distances are in angstroms. Top) E742K: The mutation K742 (right) forms one hydrogen bond (cutoff—2.7 to 3.3 angstroms; represented as dashed lines) with one ribonucleotide (G5-794) of the bound RNA, whereas E742 (left) does not form any hydrogen bond with RNA. The binding affinity difference calculated using MM-GBSA was determined to be −52.86 kcal/mol for the E742K mutation. Bottom) M747K: The mutation K747 (right) forms five hydrogen bonds between the Lys residue and two ribonucleotides (C-795 and G-796) of the bound RNA and forms a salt bridge whereas M747 (left) does not form any hydrogen bond with the RNA. The binding affinity difference calculated using MM-GBSA was determined to be −56.19 kcal/mole for the M747K mutation. The numbers depict the average distance between the donor-acceptor atom pairs forming the hydrogen bonds as observed in the production run of MD simulation. FIG. 8(C) Proximity of mutated residues relevant to solvent tolerance to DNA and depiction of H-bonds in minimized Taq polymerase ternary complex. Distances are in angstroms. Top) E507K: The mutation K507 (right) forms a salt bridge and five hydrogen bonds (cutoff—2.7 to 3.3 angstroms; represented as dashed lines) with two nucleotides (DA846, DT848) of the bound DNA, whereas E507 (left) forms only one hydrogen bond, between the backbone N and one nucleotide (DG839). The binding affinity difference calculated using MM-GBSA was determined to be −62.80 kcal/mol for the E507K mutation. Bottom) S515N: The mutation N515 (right) forms two hydrogen bonds (one shown) between the amide side chain N and one nucleotide (DA840) of the bound DNA, whereas S515 (left) forms only one hydrogen bond, with one nucleotide (DA845). The binding affinity difference calculated using MM-GBSA was determined to be −22.71 kcal/mole for the S515N mutation. The numbers depict the average distance between the donor-acceptor atom pairs forming the hydrogen bonds as observed in the production run of MD simulation. (Wild type binding affinity difference for RNA vs DNA in the same MM-GBSA units was determined to be +278.37 kcal/mol.)

FIGS. 9A-9C: GC bias in coverage of GC-rich and poor genes in target enrichment for next-generation sequencing with engineered polymerases. PCR was performed with the enzymes N-7-3-B07, N-7-3-C08 and WT with and without 5% BD to amplify four templates with different GC content using gene-specific PCR primers (GSP). The pooled libraries were sequenced on an Illumina iSeq100 using a 2×150 bp paired-end sequencing protocol as described in Methods. The template mean coverage data are presented as percent. Template coverage of libraries made with the engineered enzymes in FIG. 9A) absence of BD; FIG. 9B) presence of 5% BD, compared to WT enzyme with 0% BD (WT does not detectably amplify these genes from genomic DNA in the presence of 5% BD). FIG. 9C—Melting curves and peak traces of four templates in the absence of BD with WT-Taq and presence of 5% BD with 5 selected clones N-7-3-B07, N-7-3-C08, N-7-2-E02, L3-D04-26 and L-5-2-F01.

FIGS. 10A-10C: GC bias in coverage of GC-rich and poor genes in target enrichment for next-generation sequencing with engineered polymerases. FIG. 10(A) GC-rich templates cloned in plasmids were PCR amplified together with either WT or L-5-2-F01 or N-7-3-B07 enzyme. Gene-specific primers (one set of FWD and REV for each template) were added in the PCR reactions. The PCR mix contained 7.25 U of each enzyme and 5 ng of each template. The reaction mixture contained 0.75 mM dNTP, 1 mg/mL BSA, 3.5 mM MgCl₂, 0.5 μM of each primer and BD as specified. FIG. 10(B) The PCR mix contained 1.25 U of each enzyme and 5 ng of each template. The reaction mixture contained 0.25 mM dNTP, 1 mg/mL BSA, 3.5 mM MgCl₂, 0.5 μM of each primer and BD as specified. The following PCR programs were used for both A and B: 98.3° C. for 1 min and 95° C. for 6 min, 25 cycles of 94° C. for 30 sec, 57.8° C. for 30 sec, 72° C. for 50 sec. PCR products were isolated from 1% agarose gel electrophoresis and subjected to NGS analysis. The template mean coverage data are presented as percent. FIG. 10(C)—Melt curve traces of Taq and c-Jun in the absence and presence of 4% BD with WT and two selected clones L-5-2-F01 and N-7-3-B07.

FIG. 11. Details of GC content by template region for GC-rich templates employed herein.

FIGS. 12A-12E: Amplification efficiency of engineered polymerases in the presence of cosolvent. Selected clones were used to assess the amplification efficiency of the Taq variants in varying cosolvent concentrations with multiple different templates. Equal activities (1.25 U) of each polymerase were tested in identical conditions to assess the efficiency. Representative qPCR traces of the clones (A) or Cqs (B-E) used in a real-time PCR assay are depicted. FIG. 12(A)—Taq template in 5% BD; FIG. 12(B)—c-Jun template in 0-8% BD with select clones from early screening rounds; FIG. 12(C)—c-Jun template in 0-10% BD with select clones from later screening rounds (higher denaturation temperature was applied to the later round clones). FIG. 12(D)—Representative Cqs of templates with different GC contents (Taq, c-Jun) were used to assess GC bias in the presence of 4% BD of WT and N-7-3-B07 enzymes. FIG. 12(E)—Representative Cq values of three templates with different GC content (CDN1C 78%, EGFR 60% and KRAS 40%) were used to assess GC bias in the presence of 5% BD with 2 selected clones N-7-3-B07 and L-5-2-F01. The genes were first amplified with gene specific primers and then equimolar quantity of the PCR products were amplified in qPCR with universal primers (Table 14). No bars illustrate that Cq values were not detected. See Materials and Methods for respective PCR protocols.

FIGS. 13A-13E: Effect of 1,4-butanediol (BD) on DNA melting, polymerase stability and PCR efficiency. FIG. 13(A) GC content plot of c-Jun template flanked by primers J1/J3 (376 bp) was generated by online tool (http://www.endmemo.com/bio/gcdraw.php), FIG. 13(B) In triplicate reaction, 5 μg of purified c-Jun amplicon was used to assess the effect of 0-10% BD on T_{M} of the template in a 1×PCR buffer and FIG. 13(C) change in T_{M} of DNA was plotted against BD concentration, FIG. 13(D) effect of BD on denaturation (melt curve) of engineered polymerase, and FIG. 13(E) purified polymerases after 7^{th} round of CSR were employed to assess the amplification efficiency. After qPCR, the Cq values were plotted against the BD concentration. In 0% BD, the observed Tₘ of the DNA template was 91.53±0.058° C. whereas it reduced to 84.40±0.40° C. in 10% BD. One molar BD is equivalent to 8.86%.

FIG. 14A-14C: Enhancement of RT directly from cells using solvophilic reverse transcriptases in organic cosolvent 1,4-butanediol (BD). A,B) Fluorescence-based detection of RT products from lysed cells with and without organic cosolvent, for enzymes SFM 4-6 and L5-RT1. C) Gel analysis of RT-PCR products from lysed cells with varying amounts of organic cosolvent. Synthesized partial KRAS RNA (119 nucleotides) was mixed with the following components: a—SFM4-6 (40 ng, positive control), B, C, D—5 million bacterial cells of b (WT Taq), c (SFM4-6), and d (L-5-2-F01-RT1), respectively. Four sets of these mixtures were assigned different concentrations of BD (0%, 5%, 10%, 20%). The reverse transcription (RT) reaction volume was 20 μL for each sample. The RT reaction was performed at 80° C. for 10 minutes followed by 55° C. for 60 minutes. PCR was carried out using the NEB LUNA PCR mix on 0.5 μL of the completed RT reaction mixture. The PCR results were analyzed on a 2.5% agarose gel. Notice that the bands in WT lanes are non-specific as demonstrated by their wrong molecular weight.

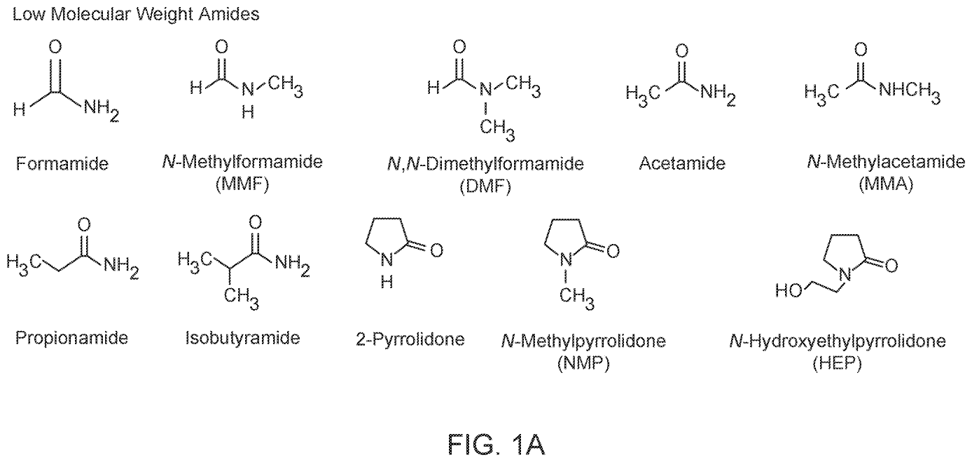

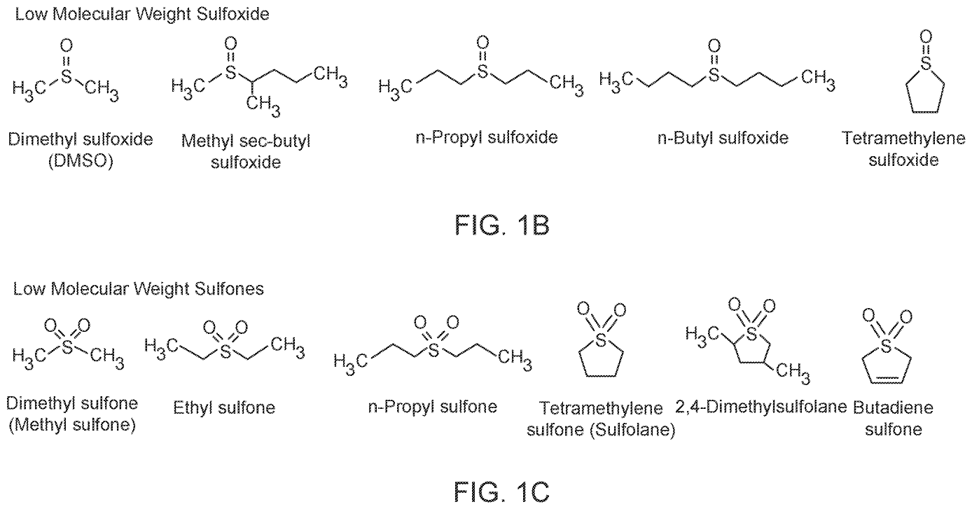

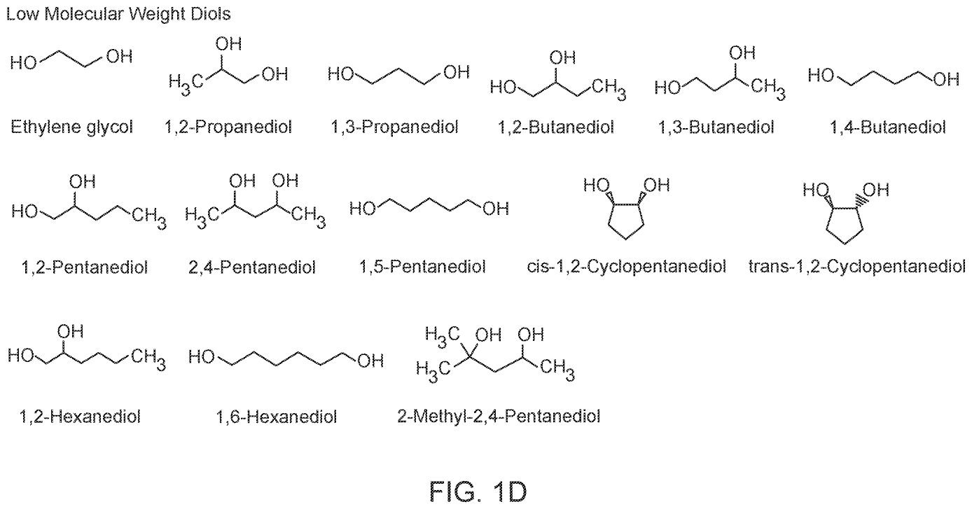

### DETAILED DESCRIPTION OF THE INVENTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

Compositions and methods for the upregulation of reverse transcription (RT) reactions and the reduction of sequence bias in RNA sequencing are presented. In one aspect, a composition for performing a reverse transcriptase reaction comprises a thermostable reverse transcriptase or a fragment thereof, a reverse transcriptase (RT) buffer, one or more template RNAs and deoxyribonucleoside triphosphates (dNTPs), and one or more low molecular weight polar organic solvents. In another aspect, a composition comprises a thermostable reverse transcriptase or a fragment thereof, a reverse transcriptase polymerase chain reaction (RT-PCR) buffer, one or more template RNAs and deoxyribonucleoside triphosphates (dNTPs), DNA dependent DNA polymerase enzyme of a fragment thereof, and one or more low molecular weight polar organic solvents. In some embodiments of compositions described herein, the low molecular weight polar organic solvents are employed as cosolvents with water or aqueous solvent.

Turning now to specific components, low molecular weight polar organic solvents, in some embodiments, are selected from the group consisting of an amide, a sulfoxide, a sulfone, and a diol. Low molecular weight polar organic solvents, in some embodiments, can have a molecular weight less than or equal to 150 g/mol. Embodiments herein are not limited to a particular organic co-solvent. Examples include but are not limited to, a low molecular weight amide, a low molecular weight sulfoxide, a low molecular weight sulfone, or low molecular weight diol. In some embodiments, the amide is selected from, for example, formamide, N-methyl formamide, N,N dimethyl formamide (DMF), acetamide, N-methylacetamide, N,N-dimethylacetamide, propionamide, isobutyramide, 2-pyrrolidone, N-methylpyrrolidone (NMP), N-hydroxyethyl pyrrolidone (HEP), N-formyl pyrrolidine, N-Formyl morpholine; delta-valerolactam, epsilon-caprolactam, or 2-azacyclooctanone; the sulfoxide is selected from, for example, dimethyl sulfoxide (DMSO), n-propyl sulfoxide, n-butyl sulfoxide, methyl sec-butyl sulfoxide, or tetramethylene sulfoxide; the sulfone is selected from, for example, dimethyl sulfone, diethylsulfone, di(n-isopropyl) sulfone, tetramethylene sulfone (sulfolane), 2,4-dimethylsulfolane, or butadienesulfone (sulfolene); and the diol is selected from, for example, 1,2-propanediol, 1,3-propanediol, 1,2-butanediol, 1,3-butanediol, 1,4-butanediol, 1,2-pentanediol, 2,4-pentanediol, 1,5-pentanediol, 1,2-cyclopetanediol, 1,2-hexanediol, 1,6-hexanediol, or 2-methyl-2,4-pentanediol. In some embodiments, the amide solvent for RT-PCR reactions is N,N-Dimethylformamide (DMF) at a concentration of about 0.5 to about 1.5 molar concentration; isobutyramide at a concentration of about 0.1 to about 1.0 molar concentration; 2-pyrrolidone at a concentration of about 0.1 to about 1.0 molar concentration; or N-methylpyrrolidone at a concentration of about 0.1 to about 1.0 molar. Alternatively, for RT reactions, the organic solvent is N,N-Dimethylformamide (DMF) at a concentration of about 0.5 to about 7.0 molar concentration; isobutyramide at a concentration of about 0.1 to about 4.5 molar concentration; 2-pyrrolidone at a concentration of about 0.1 to about 4.5 molar concentration; or N-methylpyrrolidone at a concentration of about 0.1 to about 4.5 molar.

In some embodiments, the sulfoxide for RT-PCR reactions is dimethylsulfoxide (DMSO) at a concentration of about 0.5 to about 3.0 molar concentration or tetramethylenesulfoxide at a concentration of about 0.1 to about 1.0 molar. In some embodiments, the sulfone is tetramethylenesulfone (sulfolane) at a concentration of about 0.1 to about 1.0 molar. Additionally, in some embodiments, for RT reactions, the sulfoxide is dimethylsulfoxide (DMSO) at a concentration of about 0.5 to about 7.5 molar concentration or tetramethylenesulfoxide at a concentration of about 0.1 to about 4.0 molar. In some embodiments, the sulfone is tetramethylenesulfone (sulfolane) at a concentration of about 0.1 to about 3.0 molar.

In some embodiments, the diol for RT-PCR reactions is 1,3-propanediol at a concentration of about 0.5 to about 3.0 molar concentration; 1,4-butanediol at a concentration of about 0.5 to about 2.0 molar concentration; or 1,5-pentanediol at a concentration of about 0.5 to about 1.0 molar concentration. In some embodiments, the diol for RT reactions is 1,3-propanediol at a concentration of about 0.5 to about 7.5 molar concentration; 1,4-butanediol at a concentration of about 0.5 to about 5.0 molar concentration; or 1,5-pentanediol at a concentration of about 0.5 to about 2.5 molar concentration.

In some embodiments, the one or more polar organic solvents display a rate of change of duplex DNA, DNA secondary structure, or RNA secondary structure melting temperature with respect to cosolvent concentration (dTm/d[solvent]) between −1 K/M and −15 K/M. In such embodiments, the duplex DNA corresponds to the c-jun DNA segment flanked by primers with SEQ ID NOS: 92 and 94, and where the DNA and RNA secondary structures correspond to the most stable secondary structures in the single-stranded BEGAIN DNA and RNA fragments flanked by primers with SEQ ID NOS: 126 and 127, respectively. Moreover, the one or more low molecular weight polar organic cosolvents may also display rates of change of wild-type Taq polymerase melting temperature with respect to cosolvent concentration (dT_{M}/d[solvent]) between −1 K/M and −15 K/M.

Low molecular weight cosolvents of compositions and methods described herein can be of the formula

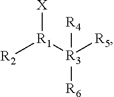

wherein:
R₁ is C or S; and
when R₁ is C, X is ═O, R₃ is N and R₆ is absent;
when R₁ is S, X is ═O or [symbol]
and R₃ is C;
R₂ is H or CH₃ only when one or more of R₄, R₅ and R₆ is not H, and otherwise R₂ is an unsubstituted or halogen-, hydroxy- or alkoxy-substituted alkyl or cycloalkyl of length m, wherein m is selected such that the total number of carbons in the compound is between 3 and 8 when R₁ is C and between 2 and 8 when R₁ is S; wherein any two of R₂, R₃, R₄, R₅ and R₆ optionally form a cyclic structure in which cyclization is effected through a bond between them; and R₄, R₅ and R₆ each is H, alkyl, cycloalkyl or halogen, hydroxy- or alkoxy substituted alkyl or cycloalkyl of length n, wherein n is selected such that the total number of carbons in the compound is between 3 and 8 when R₁ is C and between 2 and 8 when R₁ is S and when R₄ and R₅ are CH₃, R₂ cannot be H or CH₃.

In some embodiments, the one or more polar organic solvents comprises a cyclic compound, wherein the cyclization is effected through a bond between any two of R₂, R₃, R₄, R₅ and R₆. The cyclic portion, for example, can comprises five, six or seven members. In some embodiments, cyclic structure of the compound is a five, six, or seven-membered ring formed by a bond between R₂ and either R₄, R₅ or R₆. In such cyclic embodiments, R₁ can be S and remainder of the compound is unsubstituted. In some embodiments, the low molecular weight polar organic solvent comprises a compound in which R₁ is S, X is ═O or [symbol] , and R₃ is C. In some embodiments, the low molecular weight polar organic solvent is is selected from the group consisting of tetramethylene sulfone and tetramethylene sulfoxide.

Alternatively, the low molecular weight polar organic solvent is acyclic. In such embodiments, R₂ or R₃ of the compound is lower alkyl or substituted lower alkyl. In some embodiments, the polar organic solvent is selected from the group consisting of methyl sulfone, ethyl sulfone, n-propyl sulfone, n-propyl sulfoxide and methyl sec-butyl sulfoxide.

As described herein, embodiments are not limited to a particular organic co-solvent. Examples include but are not limited to, a low molecular weight amide, a low molecular weight sulfoxide, a low molecular weight sulfone, or low molecular weight diol. In some embodiments, the amide is is selected from the group consisting of formamide, N-methyl formamide, N,N-dimethyl formamide (DMF), acetamide, N-methylacetamide, N,N-dimethylacetamide, propionamide, isobutyramide, 2-pyrrolidone, N-methylpyrrolidone (NMP), N-hydroxyethyl pyrrolidone (HEP), N-formyl pyrrolidine, and N-Formyl morpholine. For example, the organic solvent can be selected from the group consisting of N,N-Dimethylformamide (DMF) at a concentration of about 0.5 to about 1.5 molar concentration, isobutyramide at a concentration of about 0.1 to about 1.0 molar concentration, 2-pyrrolidone at a concentration of about 0.1 to about 1.0 molar concentration, and N-methylpyrrolidone at a concentration of about 0.1 to about 1.0 molar.

In some embodiments, the amide solvent is N,N-Dimethylformamide (DMF) at a concentration of about 0.5 molar to about the concentration at which the melting temperature (T_{M}) of the reverse transcriptase is 5° C. higher than the optimal activity temperature of the reverse transcriptase in the absence of DMF; isobutyramide at a concentration of about 0.1 molar to about the concentration at which the melting temperature (T_{M}) of the reverse transcriptase is 5° C. higher than the optimal activity temperature of the reverse transcriptase in the absence of isobutyramide; 2-pyrrolidone at a concentration of about 0.1 molar to about the concentration at which the melting temperature (T_{M}) of the reverse transcriptase is 5° C. higher than the optimal activity temperature of the reverse transcriptase in the absence of 2-pyrrolidone; or N-methylpyrrolidone at a concentration of about 0.1 molar to about the concentration at which the melting temperature (T_{M}) of the reverse transcriptase is 5° C. higher than the optimal activity temperature of the reverse transcriptase in the absence of N-methylpyrrolidone.

In some embodiments, sulfoxides are selected from the group consisting of dimethyl sulfoxide (DMSO), n-propyl sulfoxide, n-butyl sulfoxide, methyl sec-butyl sulfoxide, and tetramethylene sulfoxide; the sulfone is selected from the group consisting of dimethyl sulfone, diethylsulfone, di(n-isopropyl) sulfone, tetramethylene sulfone (sulfolane), 2,4-dimethylsulfolane, and butadienesulfone (sulfolene). In one embodiment, the organic solvent is selected from the group consisting of dimethylsulfoxide (DMSO) at a concentration of about 0.5 to about 3.0 molar concentration and tetramethylenesulfoxide at a concentration of about 0.1 to about 1.0 molar.

In some embodiments, the organic solvent is selected from the group consisting of dimethylsulfoxide (DMSO) at a concentration range of about 0.5 molar to about the concentration at which the melting temperature (T_{M}) of the reverse transcriptase is 5° C. higher than the optimal activity temperature of the reverse transcriptase in the absence of DMSO, and tetramethylene sulfoxide at a concentration range of about 0.1 molar to about the concentration at which the melting temperature (T_{M}) of the reverse transcriptase is 5° C. higher than the optimal activity temperature of the reverse transcriptase in the absence of tetramethylene sulfoxide. In some embodiments, the organic solvent is tetramethylenesulfone (sulfolane) at a concentration of about 0.1 molar to about the concentration at which the melting temperature (T_{M}) of the reverse transcriptase is 5° C. higher than the optimal activity temperature of the reverse transcriptase in the absence of sulfolane.

Diols, in some embodiments, are selected from the group consisting of 1,2-propanediol, 1,3-propanediol, 1,2-butanediol, 1,3-butanediol, 1,4-butanediol, 1,2-pentanediol, 2,4-pentanediol, 1,5-pentanediol, 1,2-cyclopentanediol, 1,2-hexanediol, 1,6-hexanediol, and 2-methyl-2,4-pentanediol. In some embodiments, for example, the organic cosolvent is selected from the group consisting of 1,3-propanediol at a concentration of about 0.5 to about 3.0 molar concentration, 1,4-butanediol at a concentration of about 0.5 to about 2.0 molar concentration, and 1,5-pentanediol at a concentration of about 0.5 to about 1.0 molar concentration.

In some embodiments, the organic solvent is selected from the group consisting of 1,3-propanediol at a concentration of about 0.5 molar to about the concentration at which the melting temperature (T_{M}) of the reverse transcriptase is 5° C. higher than the optimal activity temperature of the reverse transcriptase in the absence of 1,3-propanediol, 1,4-butanediol at a concentration of about 0.5 molar to about the concentration at which the melting temperature (T_{M}) of the reverse transcriptase is 5° C. higher than the optimal activity temperature of the reverse transcriptase in the absence of 1,4-butanediol, and 1,5-pentanediol at a concentration of about 0.5 molar to about the concentration at which the melting temperature (T_{M}) of the reverse transcriptase is 5° C. higher than the optimal activity temperature of the reverse transcriptase in the absence of 1,5-pentanediol.

Compositions described herein also comprise a thermostable reverse transcriptase or a fragment thereof. Any thermostable reverse transcriptase consistent with the technical objectives described herein can be employed. In being thermostable, the reverse transcriptase can have an optimal RT temperature above 37° C. and preferably above 48° C. The thermostable reverse transcriptase, in some embodiments, comprises one or more non-natural amino acid alterations conferring stability and/or activity in the one or more polar organic solvents. In some embodiments, the thermostable reverse transcriptase is a mutant or modified Taq polymerase bearing at least 90% sequence similarity to wild-type Taq polymerase (SEQ ID NO: 1). In some embodiments, the amino acid sequence comprises a first set of non-natural amino acid alterations stabilizing the modified Taq DNA polymerase in an aqueous-organic medium, and a second set of non-natural amino acid alterations conferring confer reverse transcriptase activity to the modified Taq DNA polymerase.

In some embodiments, non-natural amino acid alterations of the first set stabilizing the modified Taq DNA polymerase in the low molecular weight polar organic solvents or aqueous-organic media comprising the low molecular weight polar organic solvents are selected from the group consisting of G3D, M41, L5Q, F8L, E9V, P10S, V14A, L16P, H21R, A23P, L22M, F27S, A29T, G32D, G38D, K53N, A54V, L55P, A61V, D67G, P71L, R74L, R74H, R74C, K82N, G84D, A86V, P87Q, P89S, E90D, A97T, V103A, D104G, A109V, R110Q, P114S, G115D, E117D, A118V, A118T, K128R, V136A, L149P, L162P, K171T, A180V, R183H, T186I, G187S, D191N, L193R, G195S, G200S, E201K, K202R, R205H, K206Q, G212D, S213N, S213G, N220D, L224Q, I228V, H235Y, D237G, W243R, D244E, D244V, L254P, K260N, F258S, R261H, P264S, E267K, E277G, L287Q, S290G, K292N, P302L, P302S, V310L, L311M, D320N, A326V, R328H, H333R, K346R, L351M, E363D, L365Q, P382T, N384D, E388D, T399A, A414S, A454E, A454L, A454V, A458V, L461Q, F482I, L461R, V474I, G499D A502T, 1503T, E507K, S515N, S515G, A516G, E520G, A521V, 1528T, K531R, Q534R, T539A, S543G, D551N, D551G, V586A, V586M, Q592R, L606M, A608T, S612R, 1665V, F667Y, H676L, H676R, H676Y, Q680R, E681K, K702R, A705V, V720L, V7301, D732G, D732N, E734G, V737D, V737A, S739G, V740A, V7401, E742K, F749V, F749I, F749L, K762R, K767R, L768M, E773K, L781P, E797G, E797Q, V799A, P812Q, Q782H, A814V, L813M, E825Q, and E832K of SEQ ID NO: 1.

In some embodiments, the solvostable first set of non-natural amino acid alterations are selected from the group consisting of L5Q, F8L, P10S, L16P, A23P, A29T, K31R, G38D, A61V, P89S, A97T, A118V, L162P, K171T, T186I, E201K, R205K, K206Q, G208S, K219E, N220D, I228V, M236T, D244E, D244V, R261H, D273G, L287Q, S290G, V310L, H333R, K346R, L351M, P382T, E388D, E434D, A454E, L461Q, L461R, V474I, F482I, I503T, E507K, S515N, A521V, Q534R, S543G, D551G, D551N, Q592R, L606M, A608V, S612R, H676L, Q680R, K702R, D732N, E734G, S739G, E742K, F749I, F749V, F749L, K762R, K767R, L768M, Q782H, and E832K of SEQ ID NO: 1.

In some embodiments, the solvostable first set of non-natural amino acid alterations are selected from the group consisting of P10S, L16P, A29T, K31R, G38D, A61V, A118V, L162P, T186I, G208S, N220D, I228V, D244V, D273G, S290G, K346R, L351M, E388D, A454E, L461Q, L461R, F482I, I503T, S515N, A521V, Q534R, D551G, L606M, A608V, S612R, Q680R, E734G, S739G, F749V, F749I, L768M, and E832K of SEQ ID NO: 1.

In some embodiments, the solvostable first set of non-natural amino acid alterations are selected from the group consisting of L5Q, P10S, A23P, A29T, T186I, L461R, E507K, A608V, S612R, E742K, F749L, F749I, K762R, K767R, and E832K of SEQ ID NO: 1.

In some embodiments, the solvostable first set of non-natural amino acid alterations are selected from the group consisting of P10S, L16P, A29T, K31R, G38D, A61V, A118V, L162P, T186I, G208S, N220D, I228V, D244V, D273G, S290G, K346R, L351M, E388D, A454E, L461Q, L461R, F482I, I503T, S515N, A521V, Q534R, D551G, L606M, A608V, S612R, Q680R, E734G, S739G, F749V, F749I, L768M, and E832K of SEQ ID NO: 1.

In some embodiments, the solvostable first set of non-natural amino acid alterations are selected from the group consisting of F8L, P10S, L16P, A29T, K31R, G38D, A61V, A97T, and L162P of SEQ ID NO: 1.

In some embodiments, the solvostable first set of non-natural amino acid alterations are selected from the group consisting of A186I, D244V, R205K, G208S, K219E, N220D, I228V, D273G, S290G, K346R, P382T, E388D, E434D, A454E, L461Q, L461R, V474I, F482I, I503T, E507K, S515N, A521V, Q534R, D551G, and L606M of SED ID NO: 1.

In some embodiments, the solvostable first set of non-natural amino acid alterations are selected from the group consisting of S612R, Q680R, K702R, S739G, E742K, L768M, F749I, F749V, K762R, K767R, and Q782H.

In some embodiments, the solvostable first set of non-natural amino acid alterations are selected from the group consisting of:
L30P, A54V, E434D, K206Q, S612R, V7301, and F749V of SEQ ID NO: 1;
P10S, A61V, T186I, D244V, K314R, E520G, V586A, S612R, V7301, and F749V of SEQ ID NO:1;
G12T, A54V, T186I, D244V, F667Y, and F749V of SEQ ID NO: 1;
P10S, A61V, F73S, T186I, R205K, K219E, M236T, A608V, S612R, and 2494AG of SEQ ID NO: 1;
P10S, L30P, A61V, L365P, V586A, S612R, and E832K of SEQ ID NO: 1;
P10S, A61V, D244V, S612R, and E832K of SEQ ID NO: 1;
L30P and 2494ΔG of SEQ ID NO: 1; and
A29T, G200S, D237G, and F749I of SEQ ID NO: 1.

In some embodiments, non-natural amino acid alterations of the second set conferring reverse transcriptase activity to the modified Taq DNA polymerase are selected from the group consisting of E732N, E742K, E742R, M747K, M747R, E742N, E742N, E742Q, E742Y, E742M, E742A and M747N of SED ID NO: 1.

Any combination of non-natural amino acid alterations selected from the first set stabilizing the modified Taq DNA polymerase in the low molecular weight polar organic solvents or aqueous-organic media comprising the low molecular weight polar organic solvents and the second set conferring reverse transcriptase activity to the modified Taq DNA polymerase are contemplated herein. For example, the thermostable reverse transcriptase has non-natural amino acid alterations A97T, A608V, K702R, K762R, E742K, and M747K. of SEQ ID NO:1. In some embodiments, the thermostable reverse transcriptase has non-natural amino acid alterations A97T, A608V, K702R, K762R, and E732N of SEQ ID NO:1. In some embodiments, the thermostable reverse transcriptase has non-natural amino acid alterations A23P, L162P, 228V, L461R, A521V, E734G, F749I, L768M, E742K, and M747K of SEQ ID NO:1. Additional combinations of non-natural alterations from the first and second sets are provided in Table 15 herein.

In some embodiments, the one or more non-natural amino-acid alterations can confer an increase in half-life of the thermostable reverse transcriptase of at least 50 percent at 95° C. and/or an increase in reverse transcriptase activity of at least 50% at 72° C. Moreover, presence of the one or more low molecular weight polar organic solvents can increase reverse transcriptase (RT) activity (rate of nucleotide incorporation) of the thermostable reverse transcriptase or the fragment thereof.

In another aspect, kits are provided herein. In some embodiments, a kit comprises a composition for reverse transcriptase or a composition for reverse transcriptase-PCR described herein. Low molecular weight organic solvents and reverse transcriptases of the kit can have any composition and/or properties described herein. Kits can further comprise one or more adjuvants, instructions for using components of the kit to optimize polynucleotide replication of a selected polynucleotide, oligonucleotide primers, a known template for use as a control, and one or more reaction vessels for performing the plurality of polynucleotide replication reactions. In some embodiments, the kit prescribes use of reverse transcription temperatures above 48° C. and below the melting temperature of the RNA:DNA heteroduplex in the presence of the employed concentration of the cosolvent. Moreover, the kit can prescribe use of reverse transcription temperatures above 48° C. and below the melting temperature of the reverse transcriptase protein in the presence of the employed concentration of the cosolvent. A kit, in some embodiments, prescribes preincubation at a temperature more than 5° C. below the melting temperature of the primer:template complex.

Methods of reverse transcribing RNA into DNA and methods of administering reverse transcriptase-PCR are also described herein. In some embodiments, a method of reverse transcribing RNA into DNA comprises incubating a thermostable reverse transcriptase with a reverse transcriptase buffer, a RNA template, deoxyribonucleoside triphosphates (dNTPs), DNA primers, and at least one low molecular weight organic cosolvent. The one or more low molecular weight organic cosolvents can have any identity described herein. In some embodiments, the reverse transcriptase is a modified Taq DNA polymerase bearing at least 90% sequence similarity to wild-type Taq polymerase of SEQ ID NO:1. Moreover, in some embodiments, the temperature of reverse transcription is higher than its optimal value in the absence of the polar organic cosolvent, and less than or equal to the melting temperature of the enzyme at that cosolvent concentration, minus 5° C. As detailed further herein, reaction yield or reaction efficiency can be enhanced in the presence of the at least one low molecular weight polar organic cosolvents. Additionally, reverse transcription and/or DNA amplification, in some embodiments, is administered for target enrichment in next-generation sequencing of RNA, including wherein the copy number of RNA sequences are determined by next-generation sequencing. In some embodiments, the RNA sequencing is carried out with incorporation of unique molecular identifiers (UMI) in the adapter sequences.

In another aspect, methods of detecting bacterial or viral pathogens without sample preparation, such as directly from clinical samples, are described herein. In some embodiments, a method comprises
a) incubating a clinical sample containing a virus or bacteria with RT-PCR reagents including a thermostable or solvostable reverse transcriptase (RT), one or more low molecular weight polar organic cosolvents, optionally a thermostable or solvostable DNA-dependent DNA polymerase enzyme, RT-PCR buffer, and primers complementary to the nucleic acid sequence to be detected, at a temperature exceeding 70° C. and preferably below 80° C., to lyse the virus or bacteria and release RNA without degrading the RNA;
b) incubating the lysed clinical sample at or near the optimal temperature for reverse transcription of the RT enzyme;
c) thermal cycling of the reaction mixture to PCR amplify the resulting complementary DNA (cDNA); and
d) quantification of the PCR product.
   In some embodiments, quantifying the PCR product can be administered by qPCR. The virus of the clinical sample, for example, can be SARS-CoV virus or other respiratory virus.

In another aspect, kits for detection via RT-PCR of viral or bacterial pathogen RNA directly from clinical samples without sample preparation are provided. In some embodiments, such a kit comprises a thermostable or solvostable reverse transcriptase (RT) enzyme, one or more low molecular weight polar organic cosolvents, optionally a thermostable or solvostable DNA-dependent DNA polymerase enzyme, RT-PCR buffer, and primers complementary to the nucleic acid sequence to be detected. Components of the kit, including the solvostable reverse transcriptase (RT) enzyme and low molecular weight organic solvent can have any composition and/or properties described herein. In another aspect, methods for droplet digital reverse transcription PCR (ddRT-PCR) with enhanced RT and PCR efficiency are described herein. In some embodiments, a method for droplet digital reverse transcription PCR (ddRT-PCR) with enhanced RT and PCR efficiency, the method comprising:
a) incubating RT-PCR reagents including a RT-PCR buffer, a thermostable or solvostable reverse transcriptase, a thermostable or solvostable DNA-dependent DNA polymerase, primers and/or probes for a sequence or mutation of interest, a polar organic cosolvent within emulsion droplets, and one or more RNA templates, at or near the optimal temperature for reverse transcription, such that the polar organic cosolvent at least doubles the reverse transcription yield compared to buffer lacking the cosolvent;
b) thermal cycling of the reaction mixture to PCR amplify the resulting cDNA; and
c) counting or sorting of the resulting positive droplets using a fluorescence-based counting or sorting device.

In another aspect, methods for accelerating the in vitro evolution of reverse transcriptase (RT) enzymes are provided. In some embodiments, a method for accelerating the in vitro evolution of reverse transcriptase (RT) enzymes comprising:
a) preparation of a library of thermostable polymerase enzyme gene variants;
b) expression of the enzymes corresponding to these gene variants, for example through bacterial transformation and expression or in vitro transcription/translation of the library, in individual containers, the containers preferably being either droplets or microplate wells;
c) incubation of the library of enzyme variants within individual containers, with one or more polar organic cosolvents at specified concentrations, RT-PCR buffer, primers and/or probes for an RNA sequence of interest, optionally a thermostable or solvostable DNA-dependent DNA polymerase enzyme, and the RNA template, at a temperature suitable for reverse transcription, the temperature preferably being between 48° C. and 80° C.;
d) thermal cycling of the reaction mixtures to PCR amplify the resulting cDNAs;
e) ranking of the resulting enzyme variants based on RT-PCR yield, either by screening or sorting, the screening or sorting preferably being done based on fluorescence; and
f) sequencing of the resulting top enzyme variants to identify the best RT enzymes, wherein the presence of organic cosolvent(s) at the specified concentration(s) increase the RT activity of at least one enzyme variant at least twofold above the activity in their absence. In some embodiments, at least one of the enzyme variants is a rare variant whose corresponding gene is present in the library with less than or equal to 1% frequency.

Any of the methods described herein can employ any low molecular weight polar organic solvent and reverse transcriptase enzyme described hereinabove, including modified Taq polymerases bearing at least 90% sequence similarity to wild-type Taq polymerase (SEQ ID NO: 1) and including one or more non-natural amino acids alterations conferring stability and/or activity in the one or more polar organic solvents. In some embodiments, the amino acid sequence comprises a first set of non-natural amino acid alterations stabilizing the modified Taq DNA polymerase in an aqueous-organic medium, and a second set of non-natural amino acid alterations conferring confer reverse transcriptase activity to the modified Taq DNA polymerase. Additionally, in some embodiments of methods described herein, the temperature of reverse transcription is higher than its optimal value in the absence of the polar organic cosolvent, and less than or equal to the melting temperature of the fully extended RNA:DNA heteroduplex at that cosolvent concentration, minus 5° C.

### Reverse Transcriptases in Aqueous-Organic Media

Reverse transcription (RT)—a critical step in the amplification of RNA for diagnostic (e.g., RNA sequencing) applications—is particularly prone to GC bias due to secondary structure, because the chemical instability of RNA restricts polymerization at elevated temperatures that can facilitate the reduction of secondary structures in single-stranded DNA. While next-generation RNA sequencing (RNA-seq) provides a powerful means to quantify differential gene expression patterns in high-throughput, thus playing a central role in systems biology, its accuracy is fundamentally limited by sequence bias for which the primary recourse to date has been normalization of NGS frequencies with spike-in standards and/or prior sequencing datasets. Polymerases with reverse transcriptase ability as well as organic solvent resistance could therefore be transformative in RNA-seq by enabling significantly reduced sequence bias in reverse transcription. Although retroviral reverse transcriptases have been supplanted by thermostable enzymes for many RT-PCR applications (including, e.g. TaqMan RT-qPCR with Taq polymerase for COVID testing), to date no solvent-resistant reverse transcriptases have been reported. We refer to DNA polymerases into which artificial mutations have been engineered to increase solvent resistance—by counteracting the deleterious effects of organic cosolvent—as solvostable DNA polymerases. (These polymerases preferably display rates of decline of polymerase melting temperature (T_{M}) and enzyme activity with respect to cosolvent concentration that are less than 50% of those rates for the corresponding wild-type enzyme without any solvent-resistant mutations, with enzyme activity persisting at cosolvent concentrations at least twice as high as those tolerated by the wild-type enzyme, for at least one of the aforementioned organic cosolvents. They also preferably display a greater margin of improvement in thermostability and/or activity to the parent enzyme in the presence of at least one organic cosolvent compared to its absence.)

For the above reasons, we sought to study the effects of the aforementioned organic cosolvents on thermostable reverse transcriptase enzymes and to introduce reverse transcriptase activity in solvostable DNA polymerase enzymes. Thereupon we made the completely unanticipated discovery that reverse transcriptases (especially those enzymes whose optimal temperatures for RNA-dependent DNA polymerization are significantly above 37° C., preferably above 48° C., which is not the case for the vast majority of RTs) can be solvophilic—with polar organic cosolvents increasing rather than decreasing their catalytic activity, and often increasing the activity of engineered solvostable reverse transcriptases over 5-fold and in some cases over 15-fold under conditions conducive to the elimination of secondary structure while maintaining the chemical integrity of RNA. This is the first study on organic solvent-resistant reverse transcriptases, thus expanding the repertoire of RTs to include those that are resistant to both high temperature and organic media. More generally, it is the first to demonstrate the unanticipated result that the activity of even thermostable polymerases that have not been engineered for solvostability can be greatly upregulated by polar organic cosolvents—i.e., that a broad spectrum of thermostable reverse transcriptases are in fact solvophilic and that the use of them in polymerase-compatible mixed aqueous-organic media can greatly improve RT, RT-PCR and RNA sequencing. Upregulation of polymerase activity at or above the optimal extension temperature—such that both elevated temperature and cosolvent concentration can have beneficial effects—has never been reported and is in fact contrary to previously understood principles of thermostable protein biophysics. Even more generally, it is one of the first studies to demonstrate that catalytic activity of an enzyme can be significantly upregulated, rather than reduced, by mixed aqueous-organic media compared to aqueous media. Additionally, compositions containing both RT enzyme(s) and certain polar organic cosolvent(s) within certain preferred ranges have never been anticipated nor reported to result in upregulation of the RT enzyme(s), and as such they constitute novel compositions.

In addition to RT reactions, we conducted both RT and PCR steps of RT-PCR reactions in mixed aqueous-organic media using either a thermostable or engineered solvostable RT enzyme and either a thermostable of engineered solvostable DNA-dependent DNA polymerase enzyme. We observed further improvements compared to conventional RT-PCR using thermostable RT and PCR enzymes in aqueous media.

Our findings demonstrate high performance of reverse transcription—either in isolation or together with DNA polymerization/amplification in the context of RT-PCR—in water-miscible solvents through improvement of the enzyme stability and activity, enabling dramatic reduction of sequence bias in nucleic acid polymerization not achievable through thermal resistance alone, and showing that significant sequence biases in target enrichment for nucleic acid sequencing—which have been considered chemically unavoidable and typically addressed primarily through normalization of sequencing data—can be almost entirely eliminated by the use of such organic cosolvents (instead of only temperature), with either thermostable or engineered solvostable polymerases, as a means of nucleic acid denaturation and secondary structure reduction. In addition, it was found that since the rate of change in RT activity with respect to cosolvent concentration is positive over typically a broad range (in many cases exceeding 20% cosolvent, significantly higher than for DNA-dependent DNA polymerases), such that activity increases while RNA secondary structure decreases, the stability of the enzyme in organic cosolvents at the extension temperature becomes more important in determining the maximal cosolvent concentration for such reverse transcriptases. Moreover, since thermostable RTs generally have melting temperatures significantly above their temperatures of optimal activity, and since reverse transcription alone does not require high-temperature denaturation of duplex DNA, this allows high cosolvent concentrations to be used for maximal upregulation as well as secondary structure denaturation.

Engineered solvophilic reverse transcriptases are introduced that display activity at temperatures approaching 80° C. and over 400% upregulation of activity in the presence of organic cosolvents (up to 2000%), and overcome sequence-dependent RNA secondary structure more effectively than any other tested enzyme. Preferred engineered solvophilic reverse transcriptase compositions display optimal temperatures for activity between 48° C. and 76° C. and at least 100% upregulation of activity in the presence of appropriate concentrations of at least one polar organic cosolvent. In addition, some of these enzymes are also solvostable DNA-dependent DNA polymerases, enabling their use in one enzyme, one pot RT-PCR reactions in mixed aqueous-organic media comprising certain polar organic solvents within specific concentration ranges. By expanding the scope of solvent systems compatible with nucleic acid polymerization, these inventions enable a dramatic reduction of sequence bias, with significant implications for a wide range of applications including NGS sequencing, differential gene expression analysis, synthetic biology and molecular information processing.

We used ultrahigh-throughput droplet-based selection and deep sequencing along with computational free energy and binding affinity calculations to evolve wild-type (WT) Taq polymerase (SEQ ID NO: 1) into solvophilic RNA-dependent DNA polymerases that are highly active in the presence of polar organic cosolvents, resulting in over 20% solvent resistance and over 100-fold higher stability (half-life) in the presence of 1,4-butanediol (BD), as well as tolerance to 10 times higher concentrations of the potent cosolvents sulfolane (sulfone family) and 2-pyrrolidone (amide family). DNA-dependent DNA polymerase activity was used as a screening tool to identify solvostable mutations that would be compatible with RT activity. These engineered solvostable polymerases can successfully RT-PCR a broad spectrum of recalcitrant GC-rich templates containing regions with over 90% GC content and demonstrate dramatically reduced sequence bias in the amplification of genes with varying GC content in target enrichment for next-generation sequencing (RNA-Seq). RT-PCR can be carried out in one pot using a separate solvostable DNA-dependent DNA polymerase, or even in some cases with a single enzyme that is both a solvophilic RT and solvostable DNA-dependent DNA polymerase.

We identified polymerase variants which can tolerate 30+% v/v of the most potent organic cosolvents including BD, thus enabling the use of higher concentrations of organic cosolvents to destabilize RNA secondary structures (as well as duplex DNA and single-stranded DNA) while minimizing the negative effects on the polymerase's thermostability and DNA-dependent DNA polymerase activity, and in fact dramatically improving the polymerase's RNA-dependent DNA polymerase activity by over 5-fold and in some cases between 15-20 fold. These variants can be used for amplification of GC-rich templates that routinely arise in biotechnological and diagnostic applications including next-generation DNA sequencing, and RNA-seq (where carrying out reverse transcription in the presence of organic media can reduce secondary structure). We report herein engineered polymerases that are solvostable (resistant to polar organic cosolvents) in DNA amplification reactions and solvophilic (preferring polar organic cosolvents) in reverse transcriptase reactions, with RT activities at temperatures near 70° C. in 10-20% organic cosolvent that are equal to or greater than those of state-of-the-art thermostable RTs at 55° C. in water, even on unstructured poly-A templates. Moreover, unlike all other RTs tested herein—including previously reported, highly thermostable Taq variants with RT activity—our engineered solvophilic RTs are able to synthesize cDNA from GC-rich templates in these media at elevated temperatures.

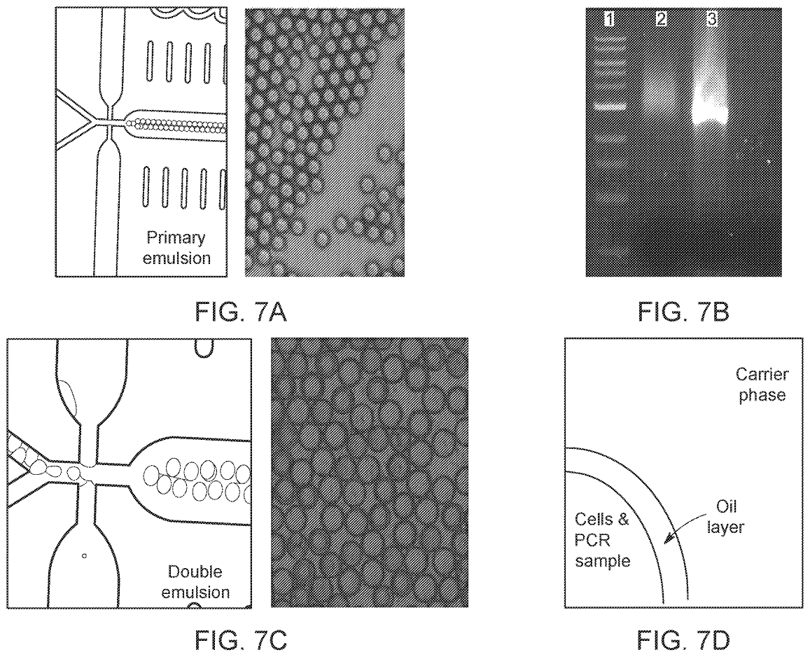

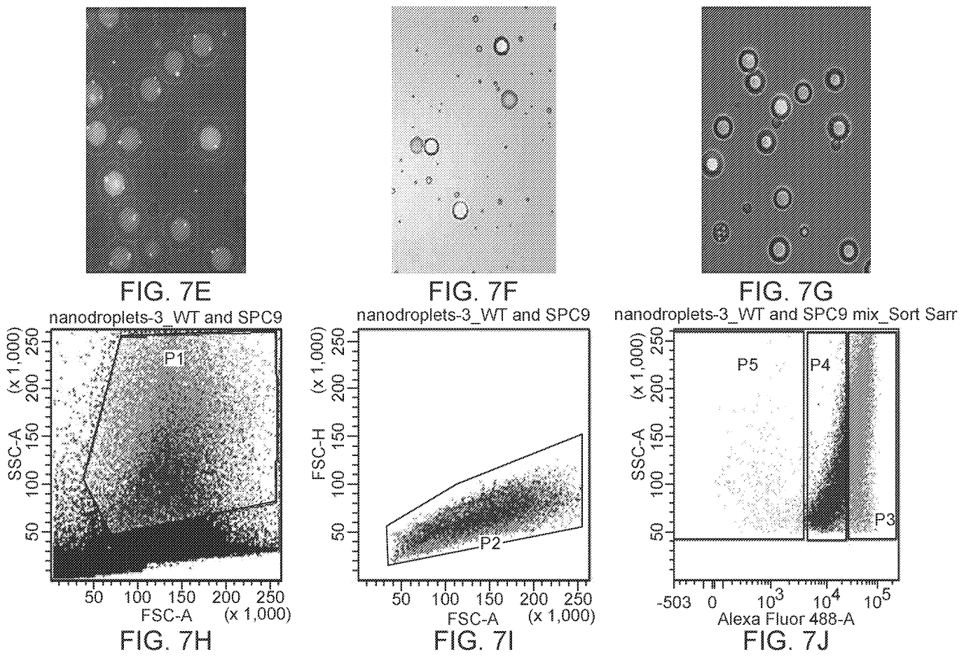

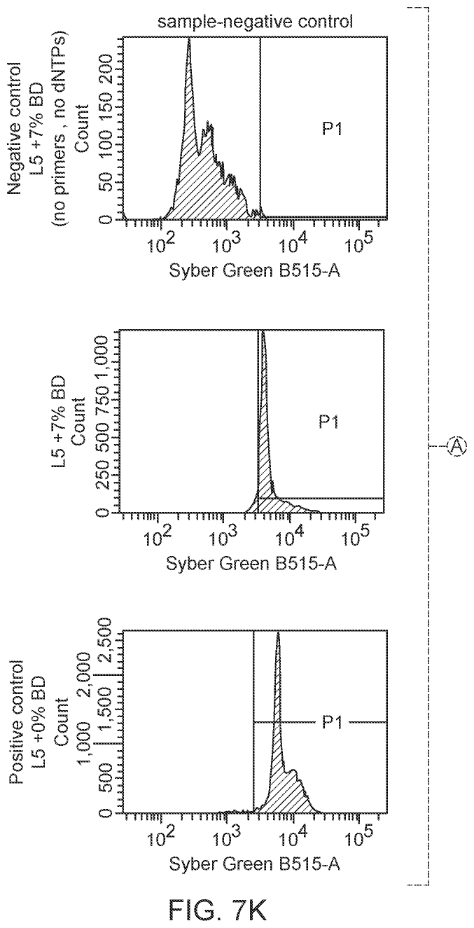

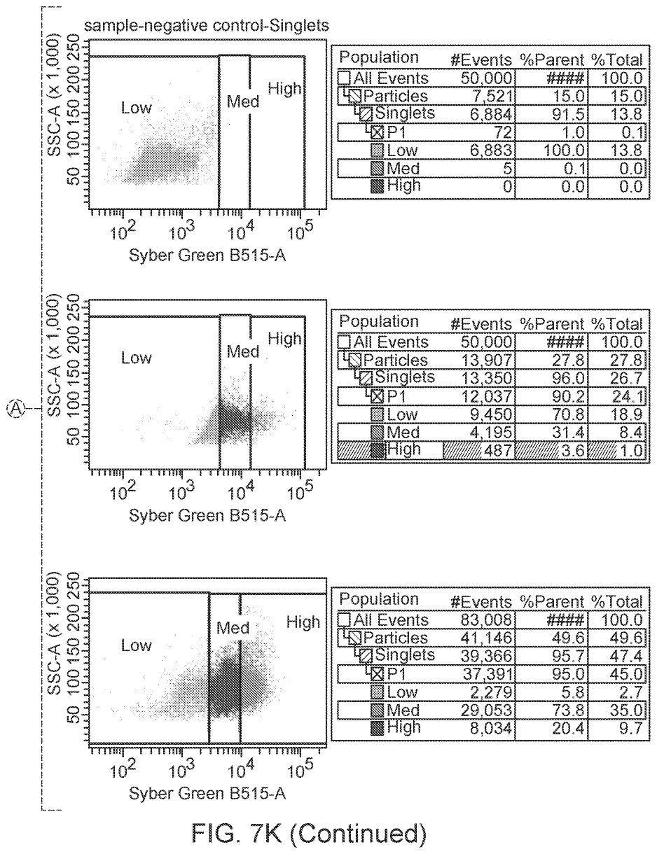

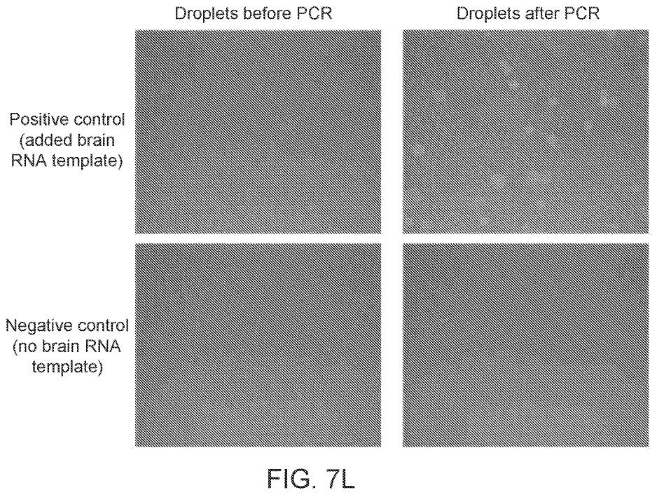

We employed microfluidics to ensure highly monodisperse water-in-oil microemulsions and to create water-in-oil-in-water microemulsions in the presence of organic cosolvents, for fluorescence-activated droplet/cell (FACS) sorting of polymerase variant libraries and for a secondary FACS-based screen confirming the superior performance of the best polymerases vs WT (FIG. 7), prior to purification and characterization. We conducted seven rounds of enrichment PCR within such droplets and followed each round by NGS library sequencing to assess the enrichment of functional clones.

Additional applications of solvophilic RT technology include: a) infectious disease detection without sample preparation steps by RT-PCR using thermostable and/or solvostable RT enzymes in the presence of organic cosolvents, due to the ability to lyse bacteria and viruses at lower temperatures conducive to the chemical stability of RNA through the destabilizing effects of organic cosolvents on these microorganisms; b) droplet digital RT-PCR (ddRT-PCR) with improved detection of rare RNA mutations using thermostable and/or solvostable RT enzymes within droplets in the presence of organic cosolvents (exploiting the greatly enhanced RT enzyme activity in these cosolvents) and droplet sorting/counting by methods such as FACS; and c) ultrahigh-throughput RT enzyme engineering with greatly enhance signal enhancement from rare active library variants due to the ability of organic cosolvents to multiply RT activity many fold. The present data demonstrate that the polymerases and/or cosolvents display the necessary and sufficient properties to enable these applications.

### Organic Cosolvent-Induced Upregulation of Reverse Transcription (RT) and RT-PCR by Thermostable Reverse Transcriptases

Cosolvent-induced activity enhancement of thermostable reverse transcriptases (i.e., reverse transcriptases whose optimal temperature for RNA-dependent DNA polymerization is above 37° C. and preferably significantly above that temperature—preferably above 48° C.) is a powerful means of reducing nucleic acid secondary structure without reducing—and instead increasing—enzyme catalytic rates. There are several possible reasons for the dramatic improvement of RT activity in BD, including improved processivity as well as solvent-induced enhancement of enzyme flexibility, but these are entirely unprecedented in the literature.

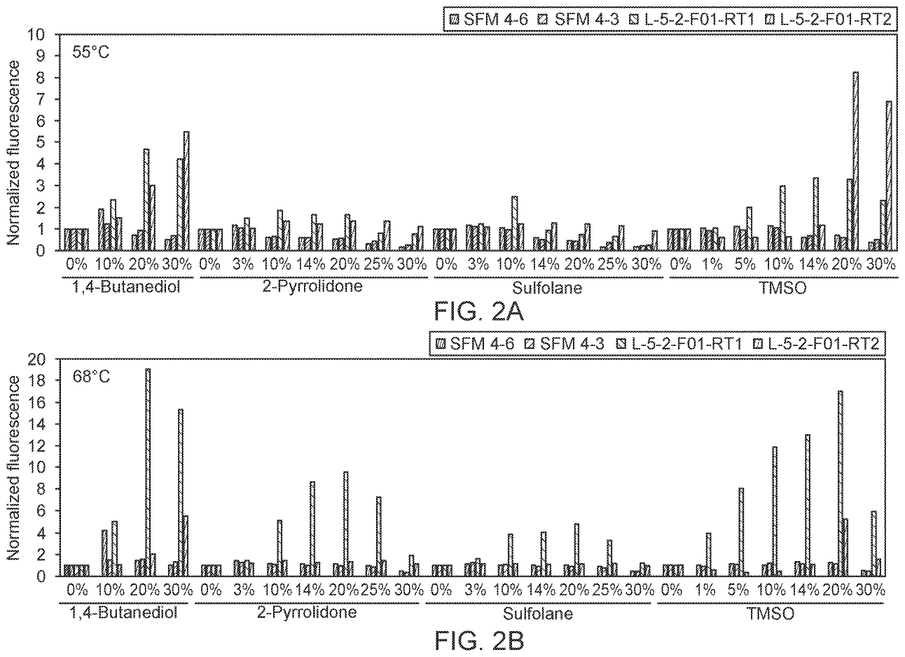

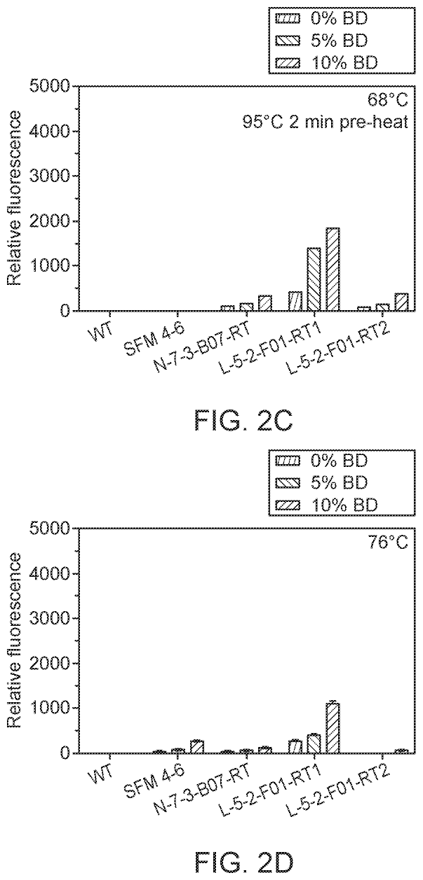

Reverse Transcriptase (RT) activity assay: Stoffel fragment mutants of Taq polymerase (SFM 4-6, SFM 4-3) were previously reported to have RT activity. Reverse transcriptase activities of highly thermostable RTs of interest were tested at different extension temperatures conducive to the reduction of RNA secondary structure (55° C., 68° C., 72° C. and 76° C.). RT activity was observed in SFM 4-6, SFM 4-3 and Taq polymerase RT variants engineered for solvent tolerance [N-7-3-B07-RT (SEQ ID NO: 4), L-5-2-F01-RT1 (SEQ ID NO: 2), and L-5-2-F01-RT2 (SEQ ID NO: 3)], and it was found that RT activities of tested mutants were dramatically enhanced in the presence of cosolvents up to over 20% v/v (FIG. 2).

Polar organic cosolvents from all the major aforementioned families—including 1,4-butanediol for the diol family, sulfolane for the sulfone family, tetramethylene sulfoxide for the sulfoxide family, and 2-pyrrolidone for the amide family—were applied in reverse transcriptase activity assays (FIG. 2, Tables 1,2).

The RT activity decreased with increasing temperature (FIG. 2 (B,D)). At 76° C. (FIG. 2D), SFM4-6 lost its RT activity whereas L-5-2-F01-RT1 was still RT active. Among all the tested enzymes, L-5-2-F01-RT1 provided RT activity which increased the most in cosolvents—almost 20× in 20% BD at 68° C. FIG. 2 (C) depicts how after the pre-heat treatment (95° C. for 2 min), SFM4-6 lost its entire RT activity even in the presence of 10% BD. Our mutants maintained some level of RT activity. Even reverse transcriptases with very limited catalytic activity in water can become highly active in mixed aqueous-organic media (FIG. 2), underscoring the important role of organic cosolvents in inducing optimal RT activity, alongside RT activity-conferring mutations.

The highest RT temperature tested for L-5-2-F01-RT1 (76° C.) exceeds the maximum temperature for activity of almost any other RT reported to date, with the exception of an engineered Tgo RT. Higher temperatures are not as useful for RNA due to chemical instability; as such, RT in organic cosolvents and slightly lower temperatures is optimal for the majority of applications, including RNA-seq. The specific RT activities of our top mutants are summarized in Table 1 with additional details reported in Table 2.

The following important observations are to be noted based on the data in FIG. 2:
Thermostable RTs (SFM 4-3, SFM 4-6):
1,4-butanediol strongly activates SFM enzymes including at high concentrations around 10% v/v, especially SFM 4-6;
3% pyrrolidone and sulfolane activate the SFM enzymes;
The extent of such activation is generally greater at 68° C. than at 55° C.;
Solvostable RTs (L-5-2-F01-RT1 and L-5-F01-RT2):
Enhancement of RT activity by cosolvents is generally significantly greater for solvostable RTs than for RTs that are only thermostable and not solvostable;
Almost 20× activation L5-RT1 is possible at 68° C. (by 20% BD) and almost 10× activation of L5-RT2 is possible at 68° C. (by 20% TMSO). 20% TMSO activates L5-RT2 at 55° C. by almost 14×;
The maximum activations by 2-pyrrolidone and for sulfolane are also much higher for L5-RT1 at 68° C. vs 55° C.;
While activities at 55° C. in the absence of cosolvent are generally higher than at 68° C., in the presence of cosolvent due to the greater extent of activation at higher temperatures, RT activities at 68° C. can routinely exceed those at 55° C. in the absence of cosolvent by a significant margin—thus enabling the use of higher RT activities together with reduced RNA secondary structure.

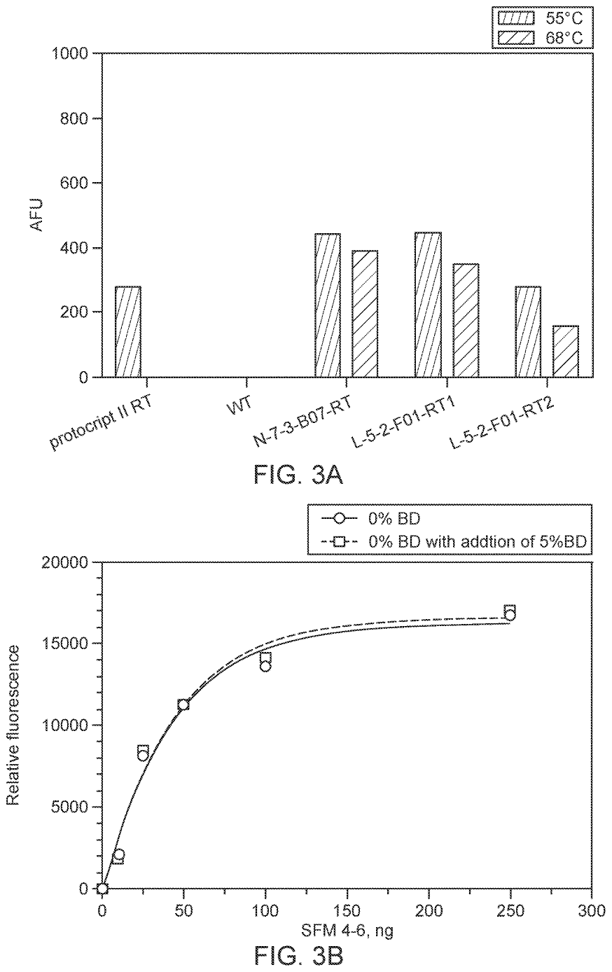

In addition, we found that the RT activity of all engineered polymerases improved dramatically at 0.1× the template concentration used in steady state RT activity assays (FIG. 3A)—a condition relevant to cDNA synthesis from mRNA using gene-specific primers—with N-7-3-B07-RT and L-5-2-F01-RT2 displaying activity nearly as high as L-5-2-F01-RT1. We note that RT activity at <=50° C. (e.g. 48° C. for Protoscript II) is almost entirely unaffected (data not shown) by cosolvent (5% BD); thus only highly thermostable RTs can fully exploit cosolvent-induced activity enhancement.

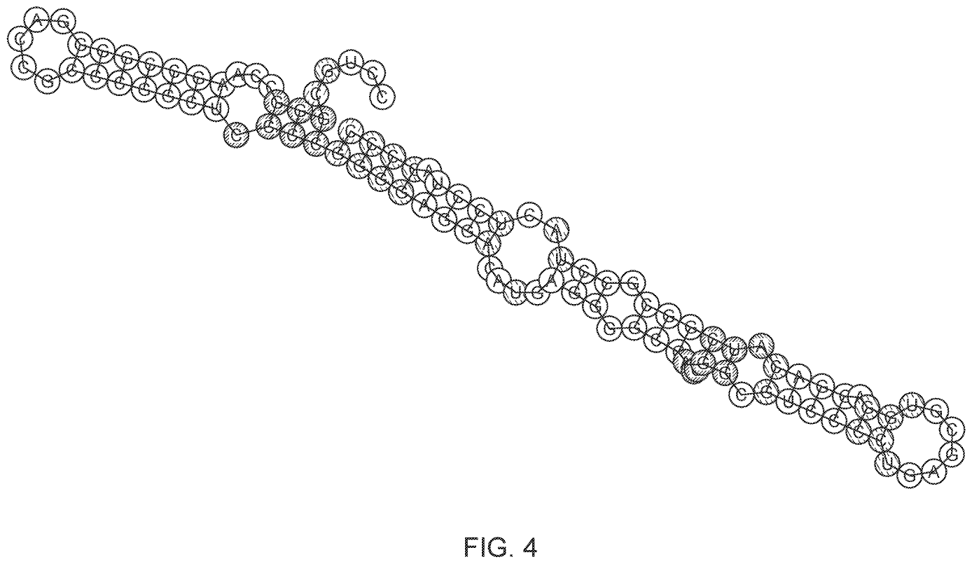

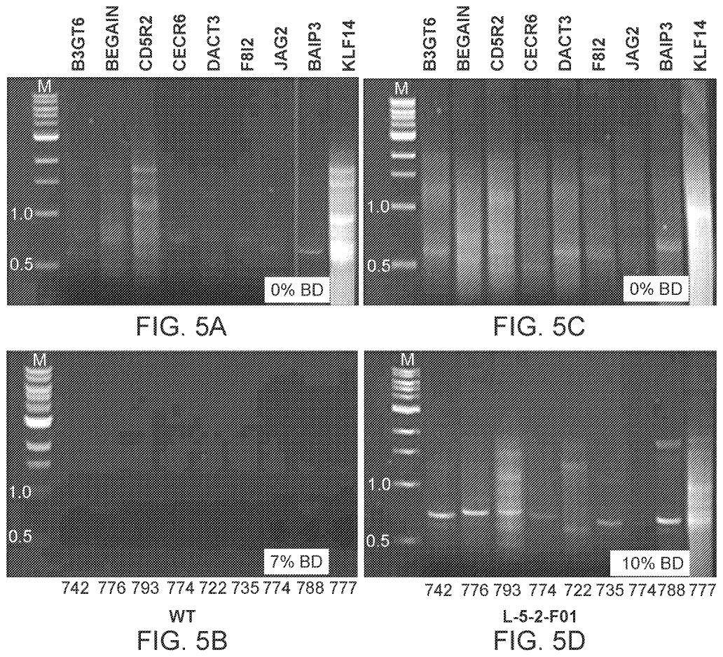

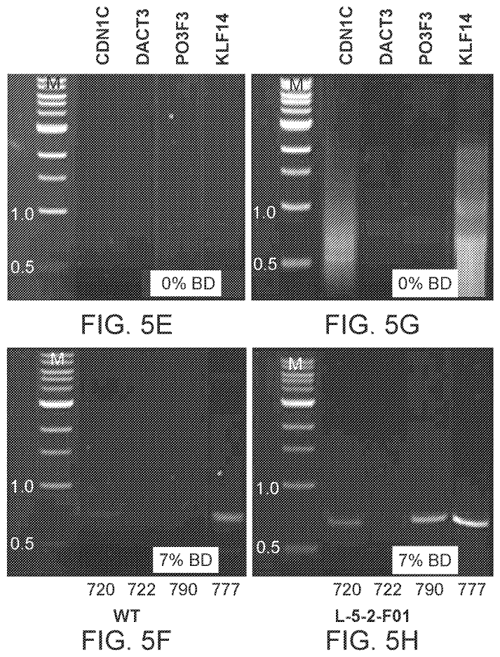

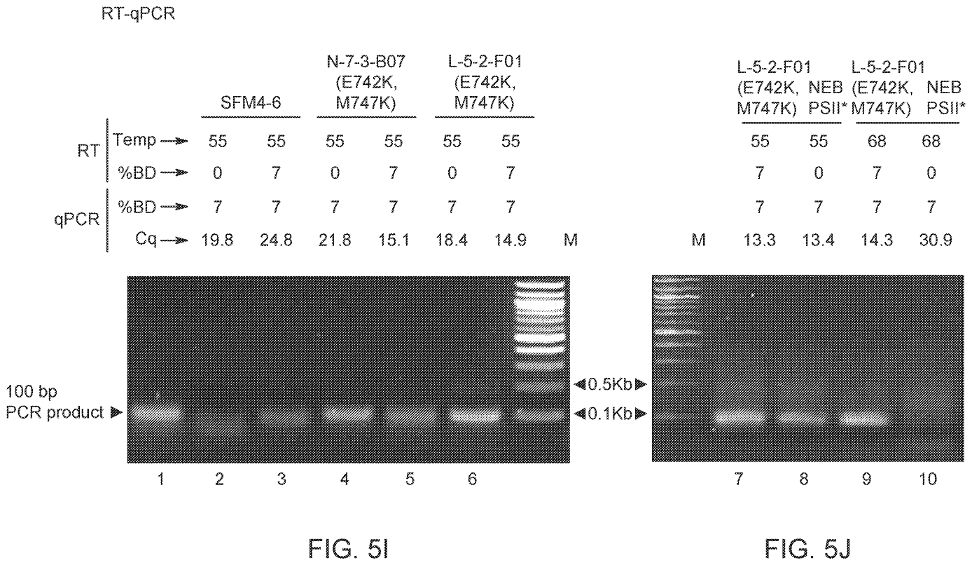

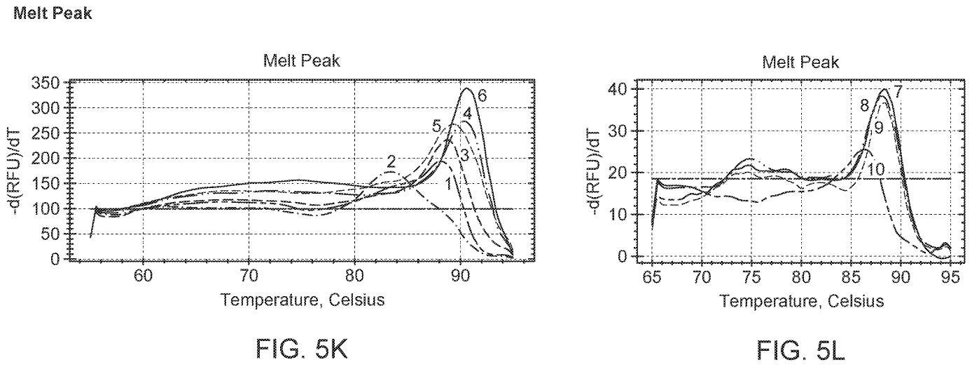

RT-qPCR assay: The most solvent-resistant polymerases displaying RT activity, L-5-2-F01-RT1 and N-7-3-B07-RT, were tested in RT-qPCR assays on a 100 base GC-rich RNA fragment (human gene BEGAIN, 75% GC; FIG. 4) and compared to SFM4-6. First, RT reactions were carried out with the respective enzymes in the presence or absence of BD and second, to directly compare the RT activities, the qPCRs were all carried out by L-5-2-F01 in the presence of 7% BD (FIG. 5 (I,K)). In 0% or 7% BD, the RT activity of SFM4-6 was limited or undetectable on this template, while the RT activities of L-5-2-F01-RT1 and N-7-3-B07-RT were enhanced in 7% BD (Cq ^{~}15; FIG. 5I, lanes 4&6) compared to 0% BD (Cq ^{~}20; FIG. 5I, lanes 3&5). Since the expected PCR product contains 75% GC, the T_{M} is around 90° C. (FIG. 5K, peaks 4&6), while the non-specific bands generated by SFM4-6 led to lower temperature melt peaks in both 0% BD and 7% BD (FIG. 5K, peaks 1&2). A comparable RT-qPCR assay by the top mutant, L-5-2-F01-RT1, was also tested against a commercial RT enzyme, ProtoScript II (a thermostable MMLV variant, like several modern RTs), at 55° C. and at much higher temperature, 68° C. FIG. 5 (J,L) (band intensities, Cq values and melt peaks) clearly show that RT activity of L-5-2-F01-RT1 with 7% BD is comparable (albeit with greater specificity, see Table 2) to ProtoScript II at 55° C. (Cq ^{~}13; lanes 7&8), but while L-5-2-F01-RT1 still retained RT activity at 68° C. (Cq ^{~}14; lane 9), ProtoScript II product was undetectable (Table 2) and led to lower temperature nonspecific melt peaks (Cq ^{~}31; lane 10). This result demonstrates the advantage of the mutant L-5-2-F01-RT1 compared to a state-of-the-art thermostable MMLV enzyme in reverse transcription of a high GC gene RNA at much higher temperature while also exploiting the advantages of cosolvent-induced enzyme upregulation and secondary structure reduction.

The results in FIG. 5, FIG. 2 and Table 2 demonstrate for several tested thermostable reverse transcriptases (L-5-2-F01-RT1, L-5-2-F01-RT2, SFM4-6, SFM4-3 . . . ), based on variants of the thermostable Taq polymerase) that these enzymes not only display resistance in their RT activity to organic cosolvents (including in the context of RT-PCR), but also show for the first time that the activity of highly thermostable RTs can be improved by the presence of organic cosolvents, with the RT activity of L-5-2-F01-RT1 increasing almost 2000% in the presence of 20% BD (FIG. 2) (and even highly thermostable RTs which are not engineered for solvostability, like SFM4-6, also display solvophilicity in their RT activity).

Although the RT activity of L-5-2-F01-RT1 in water drops between 55° C. and 68° C., due to the rate enhancement in BD that increases with temperature the activity at 68° C. in the presence of 10-20% BD is far higher than that at 55° C. in 0% BD, and moreover, is even higher than that at 55° C. in the presence of 10% BD (FIG. 2). The results in FIG. 5, FIG. 2 and Table 2 demonstrate that reverse transcriptases engineered for function in organic cosolvents (using methods described in subsequent sections) display activity up to nearly 80° C. in both aqueous and mixed aqueous-organic media, and even more significant enhancements of RT activity in organic media compared to aqueous media. By contrast, almost none of the current state-of-art RT enzymes display activity above 70° C. even in aqueous media.

Thus, with additional synthetically introduced mutations, solvostable and thermostable DNA polymerases are also solvophilic and thermostable reverse transcriptases. These engineered enzymes were also successfully applied in RT-PCR experiments as the RT enzyme in first step to produce cDNA and as the DNA-dependent DNA polymerase in the second step to complete the amplification (FIG. 5 (I-L)). A fragment of the GC-rich gene BEGAIN (75% GC), which is highly structured (FIG. 4), was chosen for RT-qPCR in mixed aqueous-organic media in order to compare the efficiencies of our engineered polymerases in RT-PCR of such templates to those of others reported in the literature. The results in FIG. 5 (I-L) show that the L-5-2-F01-RT1 mutant (which is also engineered for solvostability) conducts reverse transcription of this template more effectively than any other enzyme tested. Consistent with the RT activity assay results in FIG. 2 and Table 2, the RT activities of all thermostable RTs are significantly upregulated by 7% BD.

The N-7-3-B07-RT mutant (also an engineered solvostable RT) shows reverse transcription of BEGAIN only in 7% BD. Importantly, SFM4-6 cannot carry out reverse transcription of this structured GC-rich template in the presence of 7% BD, even though its activity on unstructured poly-A templates was upregulated by BD (Table 1, Table 2) and despite the fact that its RT activity was optimized through saturation mutagenesis, unlike our enzymes. Thus the advantages of our engineered solvophilic reverse transcriptases—which are the only RTs engineered in cosolvents—are enhanced for GC-rich template reverse transcription in the presence of organic cosolvents. Due to the simultaneous benefits of greatly enhanced activity (instead of reduced activity at high temperatures), reduced secondary structure, and negligible effects of cosolvents on the chemical integrity of RNA, this is a preferred means of reducing sequence bias (compared to increasing temperature alone) that is likely to have widespread implications in RNA-seq as well as RT-PCR diagnostics, and further demonstrates the importance of engineering solvostable DNA polymerases like those reported herein for such reactions.

While the Stoffel fragment of Taq polymerase (which lacks the exonuclease domain) has been reported to be more active and stable than full-length enzyme, this is not always the case in the presence of engineered mutations, and full-length enzyme can importantly be used in probe-based assays like TaqMan assays. Nonetheless, such fragment-based thermostable reverse transcriptases (including the enzymes SFM4-3 and SFM4-6 studied herein) are useful enzymes for organic solvent-enhanced reverse transcription. In particular, any of the engineered Taq polymerase-based RTs studied herein can also be applied as the corresponding Stoffel fragment.

### Engineering of Solvophilic Reverse Transcriptases (RTs)

The unanticipated and unprecedented observations of thermostable RT enzyme activity upregulation by the aforementioned polar organic cosolvents motivated the engineering of such enzymes for further enhanced function in mixed aqueous-organic media. This was achieved by the construction of highly diversified thermostable polymerase mutant libraries wherein both RT and DNA-dependent DNA polymerase activity in organic cosolvents could be introduced and/or improved, through ultrahigh-throughput droplet-based selection, computational modeling and experimental screening and characterization. For this purpose and proof of concept, the thermostable polymerase was chosen to be Taq polymerase, but other thermostable polymerases may be used as well.

Selections were typically carried out using DNA-dependent DNA polymerase activity in droplet-based PCR reactions because of the comparative simplicity of the protocol compared to direct selection for RT activity—including the ability to apply compartmentalized self-replication (CSR) of the polymerase gene—based on the underlying observations that a) polymerase stability in mixed aqueous-organic media is the same irrespective of whether the activity in question is DNA-dependent or RNA-dependent DNA polymerase activity; and b) the native DNA-dependent DNA polymerase catalytic activity in the presence of organic solvents and at elevated is a prerequisite (though not sufficient) for RT activity in organic media as well. Subsequently, the enzymes that displayed suitable properties in mixed aqueous-organic media were subjected to additional mutagenesis guided by NGS data as well as computational biophysics modeling to introduce RT activity in the presence of organic media, and characterized for RT and RT-PCR activity.

Polymerase library preparation, microfluidic droplet encapsulation, selection, and screening: We observed that wild type (WT) Taq polymerase (SEQ ID NO: 1) cannot amplify GC-rich templates, such as c-Jun (64% GC), in the absence of PCR-enhancing additives (organic cosolvents) such as 1,4-butanediol (BD). However, such organic cosolvents deleteriously affect the polymerase itself. We used directed evolution and droplet-based selection to co-evolve reverse transcriptases displaying both enhanced stability and activity relative to Taq DNA polymerase in the presence of organic solvents.

In the initial library, we aimed to generate 3-4 mutations per sequence by error prone PCR (epPCR) to avoid any mutational overload. The epPCR mutant library was subjected to compartmentalized self-replication (CSR) selection in the presence of BD at elevated temperatures. CSR encapsulates polymerase-expressing cells within water-in-oil emulsion droplets and challenges these polymerases to replicate their own genes via PCR. We employed the microfluidic Dolomite pEncapsulator system to improve the monodispersity of emulsion droplets. The Taq epPCR library was subjected to CSR selection in the presence of 5% BD. We pre-treated the emulsions prior to CSR-PCR at 95° C. for 6 min to minimize the WT activity and the background. Under this condition, the activity of the WT polymerase is negligible and cannot produce a CSR background signal. We established that a minimum of 16 PCR cycles are needed for WT-Taq polymerase to show quantifiable amplification of a shorterTaq gene fragment employed for screening. We screened several thousand individual clones by real-time PCR at lower denaturation temperature. We ranked the clones based on their melt curve peak area. We observed a linear correlation between amount of dsDNA and the peak area, thus concluding that the peak area is a good measure of the amount of PCR product produced. Following screening, the mutations were confirmed by Sanger sequencing (generation 1, round 1; Table 3).

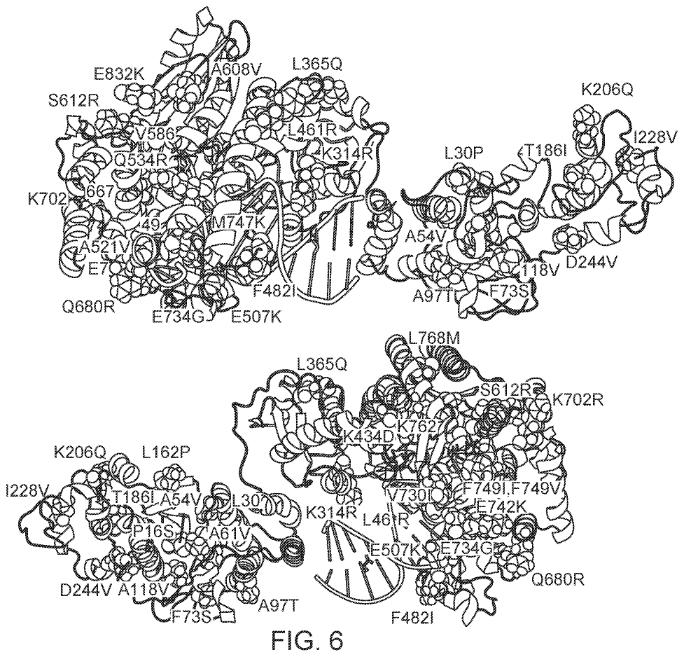

Simultaneously, to identify the best combinations of mutations, the clones selected from the first round of selection on the epPCR library were recombined by StEP PCR (shuffled library) and subjected to increased selection pressure during CSR. First, we established a CSR-PCR condition sufficient for the CSR selection in the presence of 7% BD with higher denaturation temperatures. We then ranked the clones based on melt-curve peak area as described above (Table 3). The screening positive clones were sequenced. As shown in Table 3 and FIG. 6, we found that the mutations are scattered in both 5′→3′ exonuclease and polymerase domains.

A final method of diversity generation was de novo gene synthesis of sequences (denoted SPC1-9) containing several of the top mutants from the epPCR or StEP libraries. We imposed some restrictions on the selection of the residue combinations such as surface residues sufficiently away from each other, coupled to polymerase domain residues.

NGS-guided CSR enrichment: We followed genotype redundancy as a function of CSR round by NGS. We performed seven consecutive rounds of CSR on the epPCR library (generation 1) and five rounds on the shuffled library (generation 2). After the end of each round, we analyzed the sequence diversity using NGS. We found that the frequencies of certain unique amino acid mutations progressively increased across the CSR rounds and that NGS convergence analysis enabled significant reduction in screening effort (Table 4). Convergence in terms of frequency occurred at the 7^{th} round of enrichment for the epPCR library (N-7) and the 5^{th} round of enrichment for the shuffled library (L-5); we thus designated these as the terminal libraries and carried out extensive screening on them. Our NGS data show that in the epPCR library, the mutations are randomly distributed, but after CSR, beneficial mutants are selected with high frequency, e.g., the initial frequency of F749I was 0.050 but it culminated as the most frequent mutation (35.101) (Table 4A). Similar trends were observed in the StEP libraries, e.g., amino acid position E434D has frequency 0.373 and progressively increased to 13.091 at the end of 5^{th} round (Table 4B). This variant was one of the most frequent changes we observed after Sanger sequencing. Similarly, mutations E507K, A608V, F749I, E742K were seen to enrich at each round and were present in the final screening.

We also observed that some amino acid mutants were carried over to the final round but were absent in the initial epPCR library. This phenomenon can be easily identified in the StEP library. Some of the mutants in StEP libraries acquired during enrichment CSR were previously reported, such as R205K, E434D, A608V, and E507K. Some of the mutations were enriched in the later rounds of CSR, e.g., F749I. Possibly, these mutations arose due to the error-prone nature of the Taq polymerase but enriched in the library due to their fitness to counter the selection conditions.

To validate our NGS data toward the goal of establishing NGS-based CSR convergence analysis as a methodology for assessing the optimal number of CSR rounds, we cloned and transformed the library at the end of the 1^{st}, 3^{rd}, 5^{th} and 7^{th} CSR rounds for screening. We randomly selected ^{~}250 clones and subjected them to screening for this analysis. We not only observed a near linear correspondence between NGS frequency and appearance of better performing clones—i.e., mutants which had the highest NGS frequencies in the 5^{th} or 7^{th} CSR rounds appeared most frequently in the screening of the libraries—but we also found that these mutants had higher screening scores. We also compared the performance of 1^{st} round CSR hits to the N-7^{th} round top clones. Comparative data are presented in Table 3. Top clones from enriched CSR have better performance than the 1^{st} round clones. These clones (epPCR-7^{th} CSR) not only performed better in 5% BD, but at least two of these clones (N-7-3-C08, and N-7-2-E02) also showed improvement in PCR activity in 7% BD at higher denaturation temperature (98.3° C.).

FACS-based selection and secondary screening: As a further means of quantification of the superiority of performance of polymerase variants over wild type on a common template, we used the microfluidic pEncapsulator (Dolomite Microfluidics) to prepare double emulsion (DE) droplets containing selected clones followed by sorting based on PCR efficiency using FACS. This enrichment experiment was designed to measure, using highly monodisperse droplets with a consistent number of cells per droplet, the extent to which the higher performing polymerase clones enrich with respect to WT background, which is present in a vast majority. As an example, we mixed 90% WT polymerase expressing cells with 10% SPC9 Taq variant. The WT-Taq polymerase has low activity in the presence of 5% butanediol whereas SPC9 performs better under the same reaction conditions. We prepared both primary and double emulsions using Dolomite's pEncapsulator system. Primary emulsion image data shows that the droplet populations are monodisperse with total mean diameter 20±1.2 μm (FIG. 7A) whereas we recorded 30±3.4 μm for double emulsion (FIG. 7C). During sorting, we captured approximately 1.6 million DE droplets, out of which approximately 3-4% droplets were sorted, based on SYBR Green I intensity, to identify SYBR^{HIGH}, SYBR^{MEDIUM} and SYBR^{LOW} populations. Based on number of events of sorted samples, we recovered 72.9% SYBR^{HIGH} whereas 23.8% and 1.1% SYBR^{MEDIUM} and SYBR^{LOW}, respectively. The data are presented in FIG. 7(F-J). SYBR^{MEDIUM} corresponds to negative control whereas SYBR^{LOW} represents empty droplets. We sequenced 24 clones and found that eight clones had WT-Taq genotype (33%), while 15 clones had SPC9 genotype (64%), corresponding to an enrichment of ^{~}18 fold (1:9→2:1) of the SPC9 mutant over WT-Taq. FACS was also used as alternative to CSR selection for selection of active clones from libraries, as shown in FIG. 7K for the L5 library.

We identified mutants scattered across both the 5′→3′ exonuclease and polymerase domains (FIG. 6). Many of the amino acid sites identified here are novel, and the role of F73 and E434 in the context of organic solvent resistance (including enzyme activity) also remains to be elucidated. We note that several identified mutants with improved stability contained mutations to lysine (e.g., E832K); such mutations have been reported to often improve protein stability through entropic stabilization. The mutants that are present in the polymerase domain tend to cluster in and around the substrate binding site—e.g., V586 may be implicated in DNA binding in association with E742 and A743. The residue S612 belongs to Motif A (605-617) of the polymerase domain. In general Motif A residues are mutatable, except for residue D610 which is part of the catalytic triad. Residue F667 can tolerate only tyrosine substitution and has been implicated in nucleotide substrate discrimination enabling the polymerase to incorporate dNTPs. Interestingly, we identified mutants of F749 which reside near the 0 and 01 helices of the fingers subdomain and indirectly affect the function of the enzyme.

We can hence divide our mutants into two groups: 1) those present in the 5′→3′ exo domain; and 2) those belonging to the polymerase domain. Those in category 1) such as P10, L30, A54, A61, F73, T186 etc. (Table 3) primarily affect the stability, as deletion of the N-terminal 1-288 amino acids (as in the Stoffel fragment) leads to a more thermostable polymerase domain. In this regard, we note that whereas the Stoffel fragment was reported to have a half-life approximately 2× that of WT at 97.5° C. in water, our engineered polymerases displayed up to 100× higher half-life in the presence of 5% BD at 97.5° C. (Table 5B). Even in the exo-domain, the stability hotspot is not defined. We propose that our mutants are part of a growing list of the residue positions contributing to stability toward cosolvent concentration, elevated temperature, or both. On the other hand, mutants in category 2) are often centered around the substrate binding pocket (FIG. 6). Residues in this domain may play roles in both the stability of the polymerase and in catalysis through DNA binding, dNTP discrimination etc. Reetz and co-workers have shown that although a positive correlation exists between thermostability and the organic solvent resistance of the lipase activity, the phenomena are distinct.

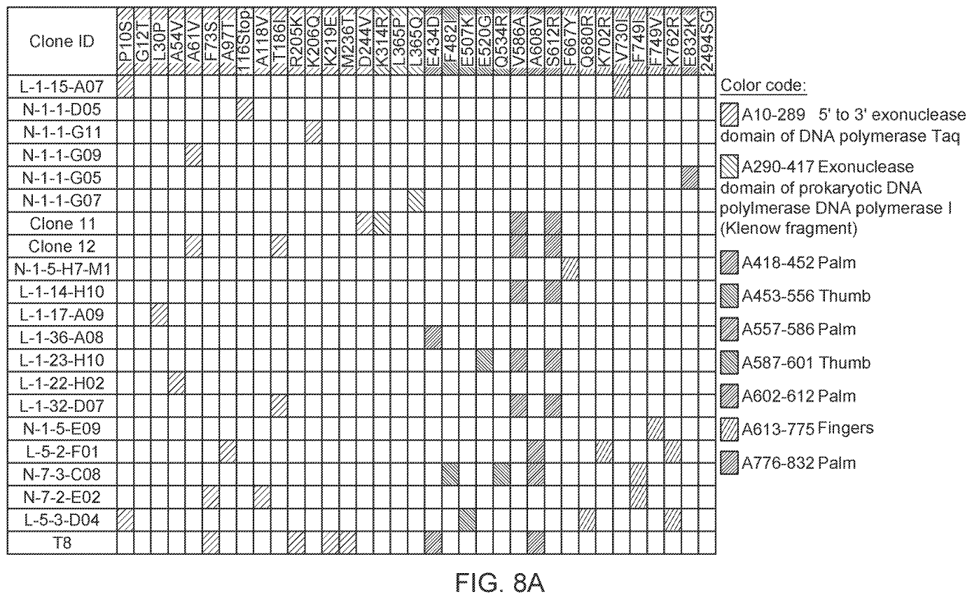

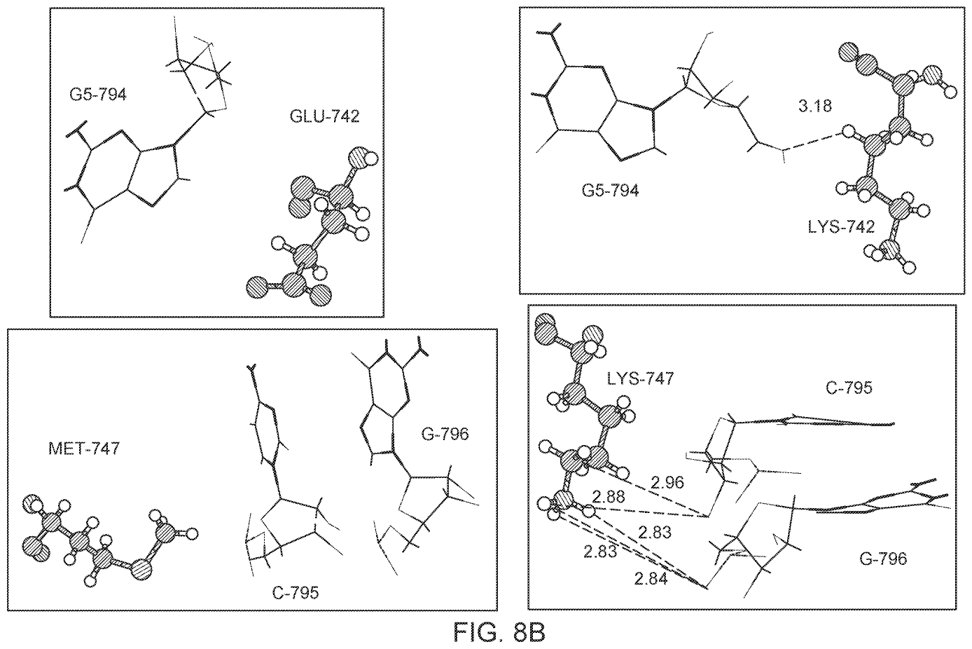

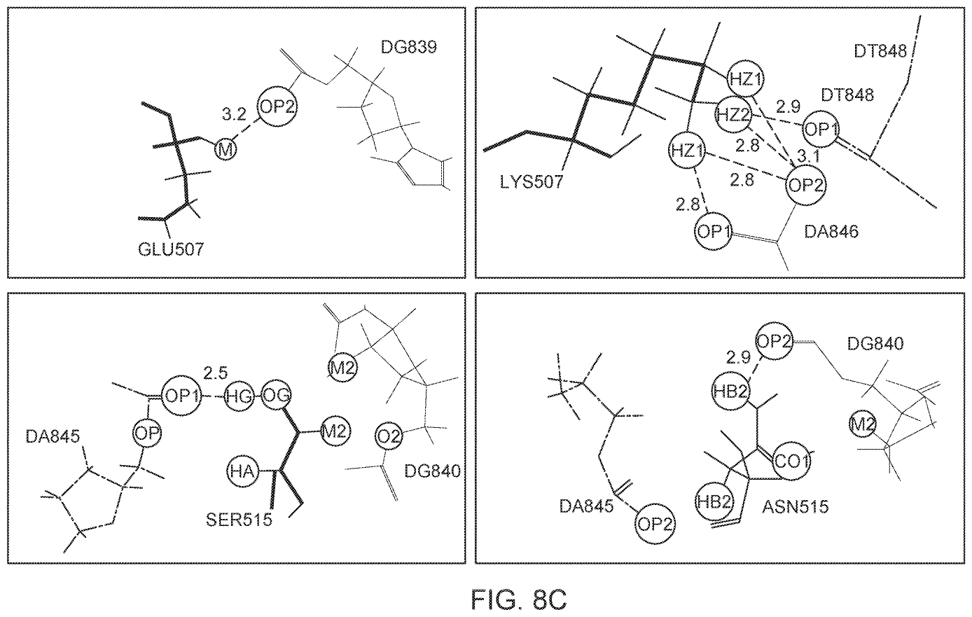

Protein purification and primer extension activity: We added N-terminal His-tag to the screening positive mutants and purified the proteins to homogeneity. We determined the polymerase activities of the WT and its mutant derivatives using self-annealing template-primer (SATP) at 72° C. We found that the specific activities of the mutants in aqueous buffer ranged from 12-210 mU/ng (Table 12). The wild-type specific activity in aqueous buffer was 119 mU/ng. In most cases we observed a loss of DNA-dependent DNA polymerase activity in 5% BD at 72° C. consistent with prior studies on wild type enzyme activity in BD, but for the engineered mutants these reductions were generally moderate as compared to the WT. One of the synthesized clones, SPC9, fully retained its activity in 5% cosolvent, demonstrating the effectiveness of synthetic design. Overall, the reduction in DNA-dependent DNA polymerase activity induced by organic cosolvents contrasts starkly with the enhancement of reverse transcriptase activity induced by the same cosolvents (Tables 1,2; FIG. 2).
Introduction of reverse transcriptase activity into engineered solvostable DNA polymerases: We synthetically introduced mutations in our best performing mutants to make them RT-active. Mutation A608V was previously identified as being conducive to RT activity, often in conjunction with F749 mutation. We observed (Table 3) A608V appearing together with E742K and F749I in more than one top screened clone from the terminal selection round of the L library (L-5-3-D10 and L-5-3-H08). The latter two mutations (often coupled) were also among the fastest enriching mutations observed in the NGS data (Table 4). Therefore, for the purpose of generating reverse transcriptases we synthetically added E742K to top clones L-5-2-F01, which also contained A608V, and N-7-3-B07, which also contained F749I. We also added M747K, which we observed through simulations to improve RNA binding (FIG. 8), together with E742K to these clones to produce the reverse transcriptases L-5-2-F01-RT1 and N-7-3-B07-RT. Separately, we added the single mutation D732N, which was reported to confer RT activity in isolation, to the top clone L-5-2-F01 to produce reverse transcriptase L-5-2-F01-RT2 (Table 1).

### Polymerase Characterization

Denaturation kinetics assessment: For stability, we used two independent methods to assess the thermal tolerance of the enzymes: 1) we calculated the t_{1/2} of the enzymes (denaturation kinetics) in an activity-based assay; and 2) we profiled thermal denaturation temperatures (T_{M}) using nanoDSF. We carried out t_{1/2} determination with His-Tag enzymes (WT and its mutant derivatives) at two different temperatures, 95° C. and 97.5° C., in the presence and absence of 5% BD by method 1. We found that the t_{1/2} of the WT in absence of organic solvent is about 41 min at 95° C. (Table 5B) which is close to the values reported previously. We observed a sharp decrease in t_{1/2} of the WT in presence of organic solvent at both the temperatures tested i.e., around 6.5-fold at 95° C. and 4.5-fold at 97.5° C. On the other hand, the mutants displayed up to three-fold higher t_{1/2} than WT at 95° C., in absence of BD, whereas up to five-fold change was recorded in the presence of BD (Table 5B).
Experimental and computational protein melting thermodynamics: Although the thermostability assay described above is well-established and widely accepted to assess the thermal tolerance of polymerases, it is a kinetic denaturation assay that depends on the primer extension ability of the enzyme. In order to assess the effect of the organic solvent on the thermodynamics of polymerase stability, we also assayed the thermal stability using nanoDSF. We determined the T_{M} of the WT and mutant polymerases (Table 6). The data show that the Taq polymerase unfolds in a two-step fashion. Our data are in agreement with the two-domain unfolding pattern reported previously for Taq polymerases, where the 5′-3′ exonuclease domain unfolds at lower temperature than the polymerase domain. As shown in Table 6, the T_{M}s of the engineered polymerases are overall higher than that of the WT, and the margins of improvement in 5% BD are highly significant. Therefore, based on two independent thermostability assays, we conclude that evolved polymerases have improved thermostability especially in cosolvents. In particular, the protein melting temperatures of four thermostable and solvostable reverse transcriptases were also measured by nanoDSF (Table 6A). The data demonstrate that the enzymes not specifically engineered for solvostability and activity in organic cosolvents display significantly lower melting temperatures—especially in the presence of organic cosolvents—than those that were engineered for function in mixed aqueous-organic media. The introduction of RT activity-inducing mutations is also observed in some cases to result in a small reduction in protein T, as well (Table 6A).

Using the MAESTROweb software, we predicted the functional consequence of the mutations on protein dynamics and stability, as shown in Table 7, which contains mutations determined to have enhanced thermostability and/or specific activity in 1,4-butanediol. Results with FoldX software were generally consistent (data not shown). To assess our ability to guide the design of synthetic solvostable polymerases via computational free energy calculations, we studied correlations between experimental T_{M} and in silico ΔΔG. Since these computational methods for folding free energy calculation assume a single unfolding step, whereas the polymerase domains unfold in separate steps, two methods for the evaluation of the predictive value of calculations were applied, to engineered polymerases from the final rounds of selection (Table 7) for which the polymerase T_{M}s were measured experimentally (Table 6). In method 1), the ΔΔG was calculated for all mutations in a sequence and then compared to the T_{M} of the polymerase domain, since the latter T_{M}s are much closer to the temperatures applied in the PCR denaturation step than the T_{M}s of the exonuclease domain are. In method 2), the ΔΔG was calculated only for the mutations in the polymerase domain under the approximation that this provides an estimate for the change in free energy of folding for the polymerase domain. The Pearson correlation between experiment and computation in method 1) was −0.57, whereas in method 2) it was −0.37 (both favorable). All but one ΔΔG computed using method 2) was predicted to be stabilizing, consistent with experimental results. For those sequences where method 1) predicted positive ΔΔGs, two predictions had statistically insignificant positive ΔΔG values (0.06 and 0.07 kJ/mol respectively, Table 7), and two such mutants (N-7-1-F06 and N-7-3-B07) were observed experimentally to destabilize the exodomain (Table 6). For the latter two mutants, we also calculated the ΔΔG of the exodomain mutations only and consistently found highly positive (unfavorable) ΔΔGs for that domain (2.92 kJ/mol and 2.00 kJ/mol, respectively). The correlation between experimentally measured exodomain T_{M}s and predicted ΔΔGs for exodomain mutations only was also favorable at −0.42. Thus like the binding affinity calculations, folding free energy calculations display good predictive accuracy and can be employed for rational library design.

Our stability data demonstrate that the selected mutations can affect thermostability quite differently in the absence and presence of cosolvent (Table 5B). One of the most heat-resistant clones, L-1-17-A09, which has mutations only in the 5′→3′ exonuclease domain, does not perform well in the presence of 5% BD. On the other hand, the clones which have moderate thermostability in 0% BD but contain mutations in the substrate binding area such as L-1-14-H10 performed better in presence of 5% BD. We observed this trend for many of the mutant polymerases.

We note that since reverse transcriptase activity of such enzymes is upregulated by organic cosolvents, the unfolding temperature of a polymerase sets an upper bound on the optimal temperature for RT activity. For example, from Table 6 it is observed that in 5% BD the melting temperature of the exodomain of Taq polymerases is around 80° C., whereas the melting temperature of the polymerase domain is generally much higher. This is consistent with the observation of highly effective RT activity at close to 80° C. (FIG. 2).

Ligand binding affinity evaluation by MM-GB(PB)SA: The impact of mutations on binding affinity to ligand (here, RNA or DNA) was evaluated by MM-GB(PB)SA. It was understood that mutations in close proximity to RNA or DNA are likely to influence ligand binding, hence all mutations in screened clones were first screened in-silico for their proximity to RNA or DNA. Five mutations were identified that had <6 Å distance from DNA/RNA—S515N, E507K, A516G, and E742K, and M747K. We prioritized four mutations—E742K and M747K for RNA, and S515N and E507K for DNA—based on proximity and nature of amino acid change.

The binding affinity energy evaluation for RNA was performed on E742K and M747K mutant complexes along with reference complex for relative comparison. The binding affinity difference calculated using MM-GBSA was determined to be −52.86 kcal/mol for the E742K mutation. The binding affinity difference calculated using MM-GBSA was determined to be −56.19 kcal/mole for the M747K mutation. Within the minimized M747K mutant complex, the lysine (K) residue was observed to be involved in forming five hydrogen bonds (hydrogen bond cut-off: 2.7-3.3 Å) and one salt bridge with the bound RNA ligand (FIG. 8B). In contrast, within the minimized reference complex, no hydrogen bond could be visualized between the reference Methionine (M) and the bound RNA ligand. In the minimized E742K complex, the mutation Lysine (K) was observed to form one hydrogen bond with the bound RNA, compared to no hydrogen bond for the native E.

M742K is located near the active site and forms a salt bridge with the nucleic acid backbone. The calculated binding affinity energy values for mutations M747K and E742K obtained by molecular dynamics and MM-GB(PB)SA, which were improved compared to WT, suggested that the in-silico evaluation was in good agreement with wet-lab results, given these mutations are found to improve RT activity by orders of magnitude. Along with M742K and E742K, A608V is one of the most potent RT activity-conferring mutations and near the active site, adjacent to D610 which binds to Mg²⁺ ions. This may explain the particularly high RT activity of L-5-2-F01-RT1, which contains both A608V and E742K mutations. Also, some of the above mutations identified from our screens have been selected for solvent compatibility in addition to their functions in polymerase activity; thus they may facilitate solvophilicity of the resulting reverse transcriptases along with other novel mutations identified.

The binding affinity energy evaluation for DNA was performed on E507K and S515N mutant complexes along with reference complex for relative comparison. The binding affinity difference calculated using MM-GBSA was determined to be −62.80 kcal/mol for the E507K mutation. The binding affinity difference calculated using MM-GBSA was determined to be −22.71 kcal/mole for the S515N mutation. Within the minimized S515N mutant complex, the asparagine (N) residue was observed to be involved in forming two hydrogen bonds (hydrogen bond cut-off: 2.7-3.3 Å) with the bound DNA ligand (FIG. 8C). In contrast, within the minimized reference complex, only one hydrogen bond could be visualized between the reference Serine (S) and the bound DNA ligand. In the minimized E507K complex, the mutation Lysine (K) was observed to form a salt bridge and five hydrogen bonds with the bound DNA, compared to one hydrogen bond for the native E.

We identified mutant S515N that has strong interactions with the nucleic acid substrate (FIG. 8C). The calculated binding affinity energy values for mutations S515N and E507K obtained by molecular dynamics and MM-GB(PB)SA, which were improved compared to WT, suggested that the in-silico evaluation was in good agreement with wet-lab results, given a) the effect of E507K results in orders of magnitude improvement in the polymerase-DNA dissociation constant; b) the mutation S515N was obtained in our mutant N-7-4-D06 clone, a hit of the terminal selection round (N1-7th) with among the highest specific activities in the presence of 5% BD. These results indicate that computational binding affinity calculations can assist in the rational design of libraries and synthetic sequences that include active site mutations.

(Wild type binding affinity difference for RNA vs DNA in the same MM-GBSA units was determined to be +278.37 kcal/mol.)

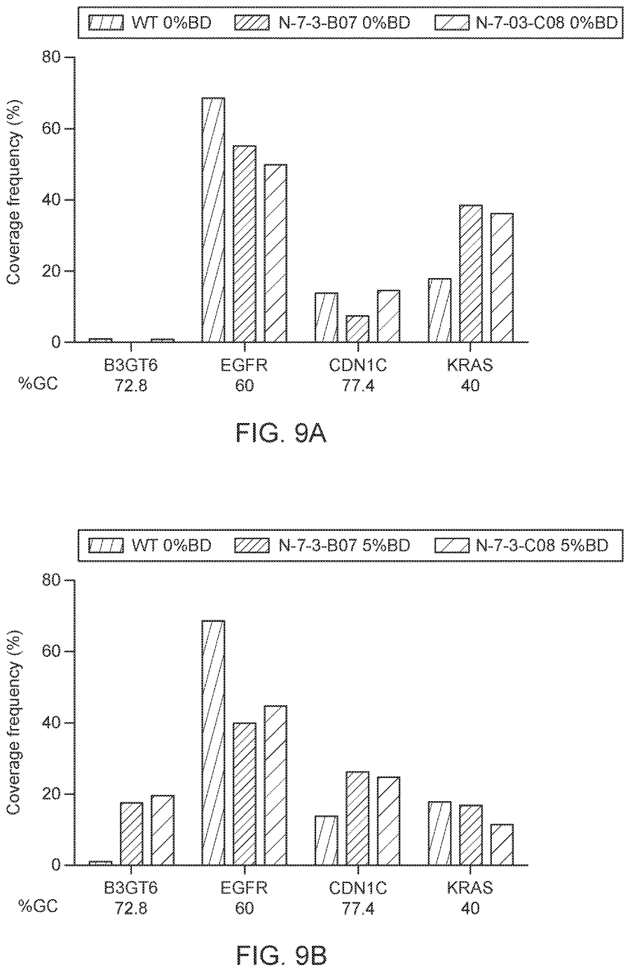

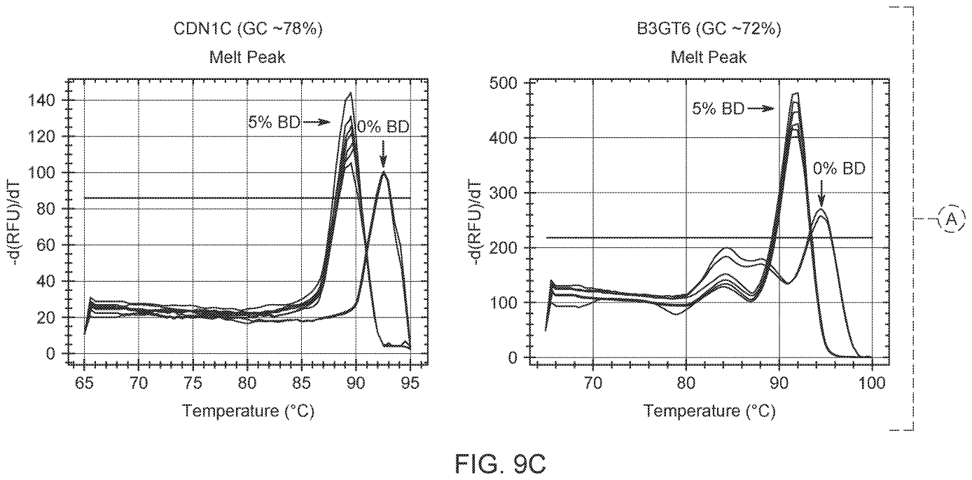

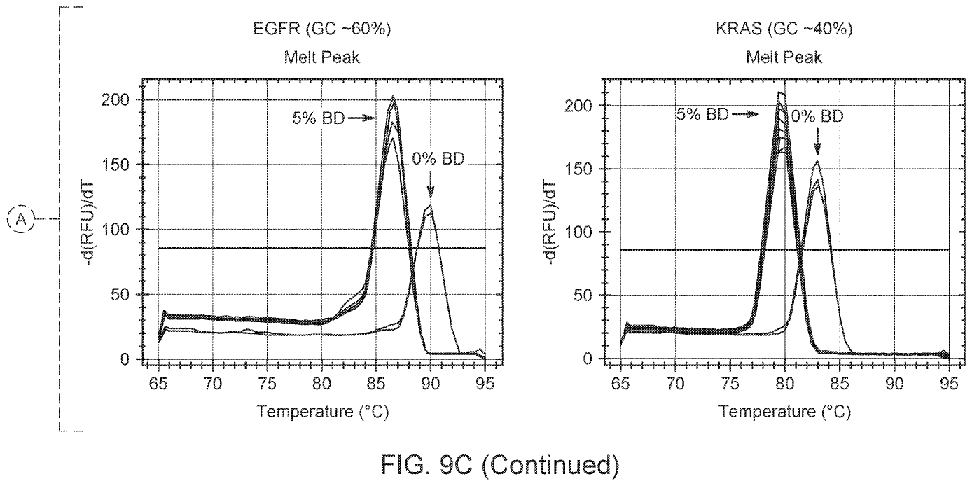

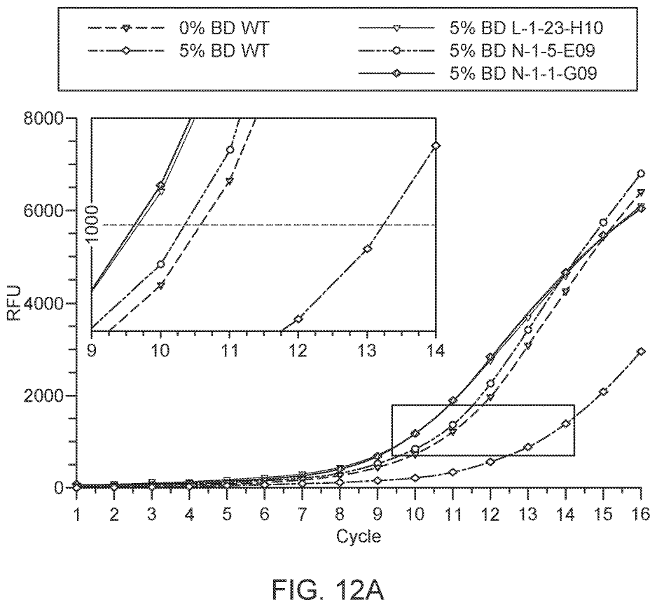

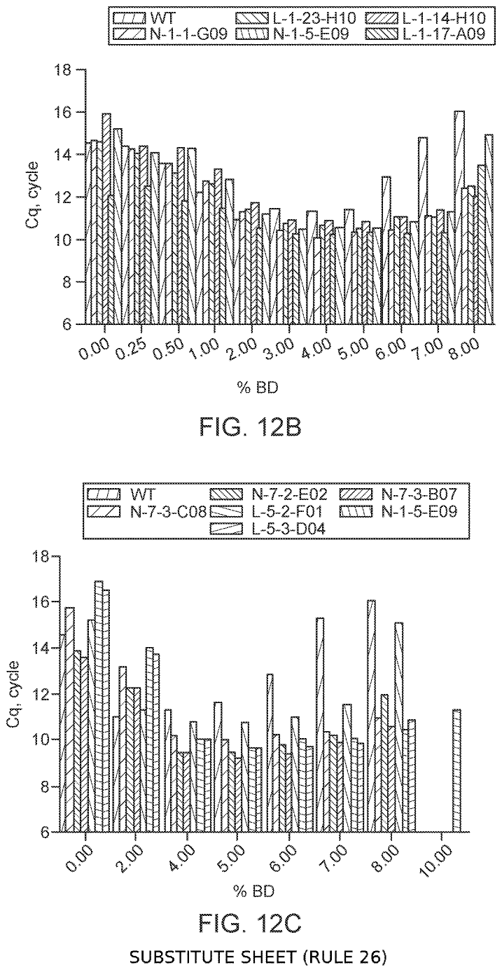

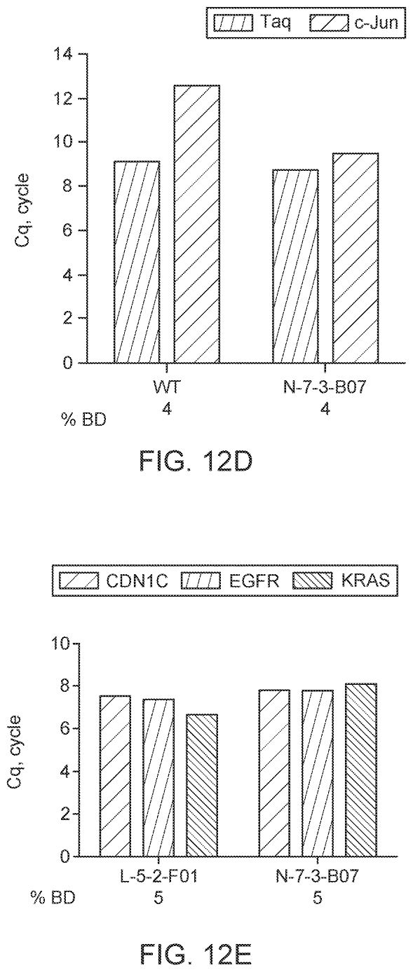

GC bias evaluation using NGS: We applied the Illumina target enrichment protocol to genes of widely varying GC content from genomic DNA. As shown in FIG. 9, without BD, there was no effect of the engineered enzymes N-7-3-B07 and N-7-3-C08 on increasing the mean coverage of GC-rich genes B3GT6 and CDN1C relative to the lower GC content genes EGFR and KRAS (FIG. 9A), with the bias being consistent with the Tₘs depicted in FIG. 9C. However, in the presence of 5% BD, while amplification efficiency of several of the genes from genomic DNA was significantly compromised for WT (data not shown), the engineered enzymes significantly increased the coverage of the GC-rich genes B3GT6 and CDN1C (FIG. 9B) in a manner consistent with the T_{M} reduction in 5% BD (FIG. 9C). This shows the effect of cosolvent in reducing GC bias when applied with the engineered polymerases. The significant reduction in GC bias is consistent with the similar Cqs reported in FIG. 12E for these genes using N-7-3-B07.

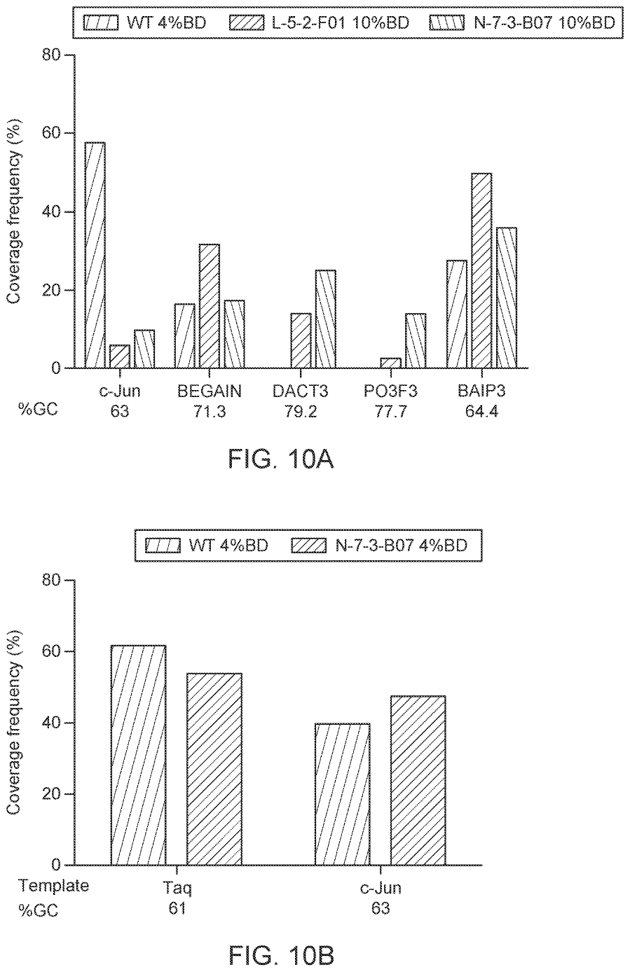

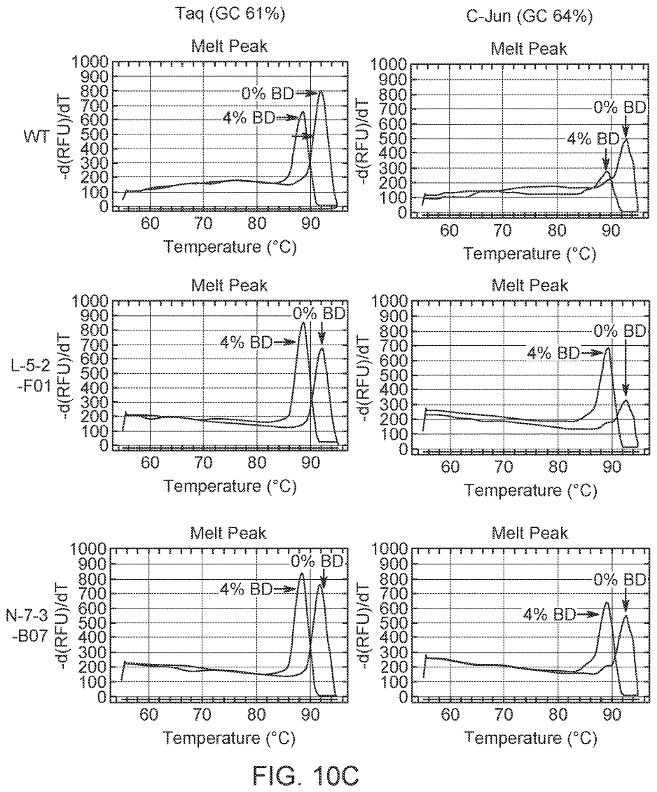

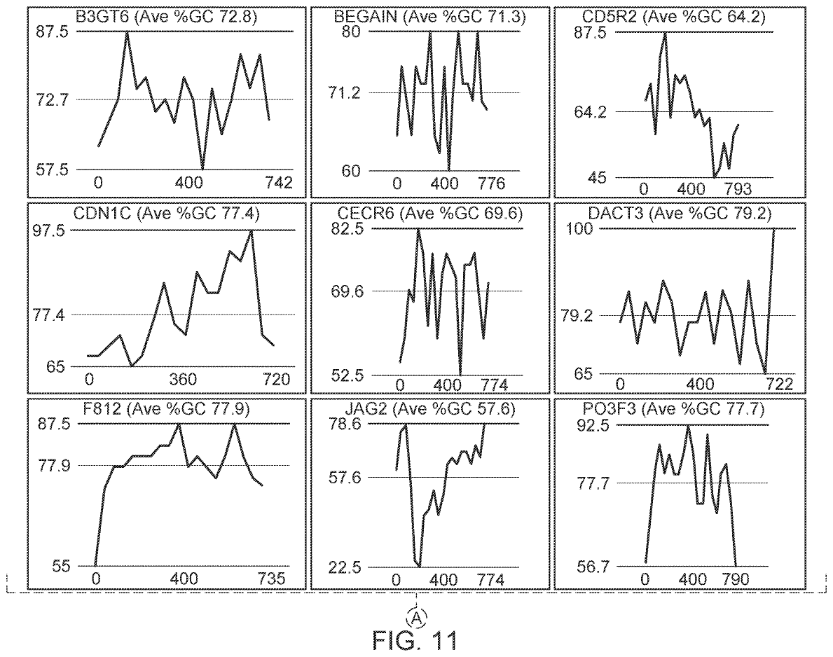

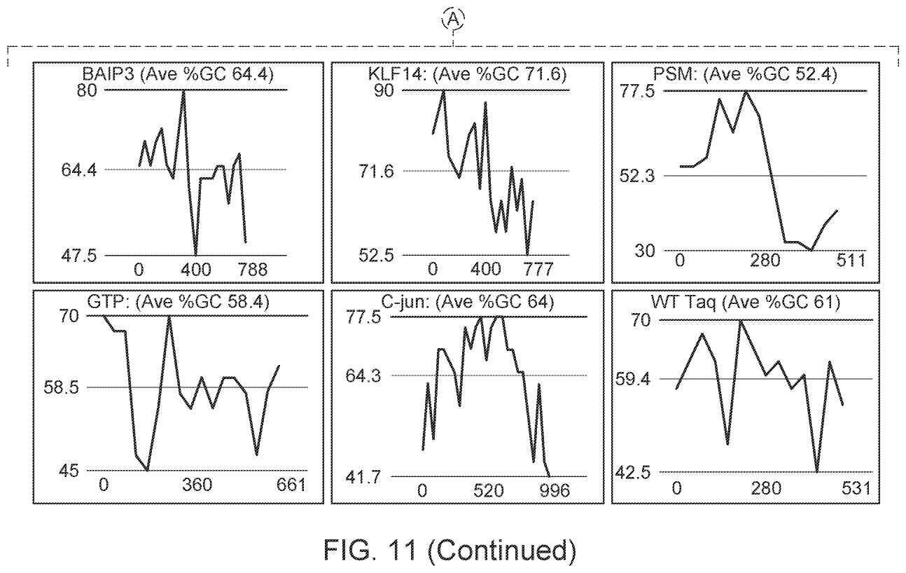

We further evaluated the GC bias of NGS target enrichment by our two best-performing mutants, L-5-2-F01 and N-7-3-B07, and compared with the GC bias of WT under high denaturation temperature conditions using an equimolar mixture of plasmid DNA along with gene specific FWD and REV primers corresponding to several of the aforementioned GC-rich genes. GC-rich templates (plasmid clones) were PCR amplified together and the purified PCR products were analyzed by NGS to see the frequency of each gene in the PCR pool (Table 8, FIG. 10). A concentration of 4% BD was added in the PCR pool when WT enzyme was used since there was no template amplification with WT over 5% BD under high denaturation temperature conditions. However, a concentration of 10% BD was added in the PCR reactions when L-5-2-F01 or N-7-3-B07 were used since as demonstrated in FIG. 5, L-5-2-F01 could amplify a number of GC-rich genes under these conditions. As shown in Table 8 Å, WT had difficulties (lower frequency) in amplifying the high GC genes (BEGAIN—the RNA for which was also very efficiently reverse transcribed by solvophilic reverse transcriptases, DACT3 and P03F3), whereas both engineered mutants were able to amplify the three high GC genes much better (higher frequency) than the WT. The higher frequencies of these genes in the PCR pool show that both engineered enzymes have the ability to lower the GC bias during amplification of GC-rich templates in applications including NGS. In a related experiment with NGS target enrichment of just the c-Jun and Taq genes (Table 8B), which have T_{M}s differing by several degrees (FIG. 10C) due to a 7% difference in max GC content (FIG. 11), N-7-3-B07 achieves several fold lower (almost negligible) bias than the WT polymerase. The much lower GC bias in NGS target enrichment for the engineered polymerases is consistent with the much smaller difference in Cqs for plasmid templates of differing GC content for N-7-3-B07 relative to WT (FIG. 12D).

It is well-accepted that GC content is the primary cause of sequence bias in NGS target enrichment by RT-PCR (in the case of RNA targets where the sequencing is referred to as RNA-Seq) or PCR (for DNA targets). Most available methods for addressing sequence bias correct for bias computationally. Use of organic cosolvents with highly thermostable (solvophilic) RTs and solvostable DNA-dependent DNA polymerases is noteworthy in that it corrects for GC bias chemically. Since it is not possible to achieve 100% DNA denaturation for many GC-rich genes in water regardless of temperature, use of engineered polymerases (including thermostable and preferably highly solvophilic RTs and solvostable DNA-dependent DNA polymerases) in the presence of cosolvents is the preferred approach for reduction of GC bias in NGS target enrichment using RT-PCR protocols. We employed two experimental methods, four mutants and a number of GC-rich genes and showed a dramatic reduction in GC bias through the use of our engineered polymerases and mixed aqueous-organic media (FIG. 9, Table 8 and FIG. 10). A polymerase's ability to reduce GC bias in NGS target enrichment is more important than high fidelity, due to error correction by unique molecular identifiers (UMIs). While molecular barcoding through UMIs (which can added to either RNA or DNA templates) is helpful for mitigating the effects of sequence bias by allowing digital counting of the frequencies of nucleic acid sequences present prior to amplification, without chemical bias correction sequence copy numbers can be incorrectly estimated by up to two orders of magnitude. Solvostable DNA polymerases reduce copy number estimation errors by orders of magnitude (FIG. 9, Table 8), and hence are expected to largely eliminate sequence bias when applied in conjunction with UMIs in genome sequencing applications.

Highly solvophilic RNA-dependent DNA polymerases L-5-2-F01-RT1 and N-7-3-B07-RT, which can be combined with solvostable DNA-dependent DNA polymerases L-5-2-F01 and N-7-3-B07, display the ability to enrich targets for NGS sequencing/RNA-Seq with dramatically reduced bias and to amplify hitherto intractable genes. For example, their ability to synthesize and amplify cDNA from GC-rich RNA like BEGAIN with much higher efficiency (FIG. 5I-L; Table 2) and the significantly lower sequencing bias against BEGAIN in NGS sequencing (FIG. 10; Table 8) demonstrate the significant utility of such enzymes and polar organic cosolvents in reducing sequence bias in RNA sequencing (RNA-seq).

PCR efficiency assays: Cq values obtained in a real-time PCR assay were used as a measure of PCR efficiency since they are unaffected by the choice of the total number of PCR cycles. A mutant in the presence of cosolvent that has the same or lower Cq value compared to the WT will be a better-performing variant. Nineteen variants from the early rounds of CSR (round 4 or earlier) that have lower Cq values in the presence of 5% and or 7% BD than the WT were identified. Representative qPCR traces are shown in FIG. 12A. The synthesized polymerase clones SPC3, 4, 5 and 9 were able to tolerate up to 7% BD and were among the best performing mutants from the early rounds.

For a GC-rich template, c-Jun, qPCR was carried out for early round mutants with increasing concentrations of BD (FIG. 12), since both qPCR and the desired property of reverse transcription in such media benefit from solvent resistance. The WT enzyme and the mutants were not able to amplify c-Jun template at 0% BD (Cq values were close to the end of the total number of cycles). In the case of the WT enzyme, lower Cq values were seen up to 4-5% BD; however, Cq values with some earlier round mutants were high beyond 5% BD (FIG. 12B) whereas some later round mutants were tolerant up to 7-8% BD (FIG. 12C), indicating that mutants were more tolerant to BD. Representative data are presented in Table 9, demonstrating further improvements in polymerases derived from later rounds compared to WT. Cq values showed all engineered mutants had much higher BD tolerance than the WT especially on the GC-rich c-Jun template with higher initial temperature treatment condition (Table 9).

FIG. 12D directly compares the dependence of Cq on GC content in the presence of 4% BD for WT and engineered polymerase N-7-3-B07. N-7-3-B07 showed very similar Cqs for Taq and c-Jun templates, whereas WT did not. Additionally, three therapeutically relevant templates (FIG. 12E) were used to assess the dependence of Cq on GC content in the presence of 5% BD with two of our high-performing mutants (N-7-3-B07 and L-5-2-F01). The selected mutants can amplify highly GC-rich templates (CDN1C 78%) with very similar efficiencies (Cq) as GC-poor templates (EGFR 60% and KRAS 40%), whereas WT cannot.

To test the efficacy and the tolerance limit of the engineered mutant polymerases (solvophilic reverse transcriptases and/or solvostable DNA polymerases) for cosolvents from other polar organic compound families in (RT-)PCR, the qPCR assays were carried out with one of the best performing mutants, L-5-2-F01 (related to the solvophilic reverse transcriptase L-5-2-F01-RT1) in the presence of different concentrations of potent cosolvents 2-pyrrolidone (amide family) or sulfolane (sulfone family) (Table 10). WT barely showed amplification of GC-rich c-Jun template in the presence of 2-pyrrolidone or sulfolane (Cq values were close to the cycle threshold), whereas L-5-2-F01 was found to have higher tolerance to both cosolvents (about 5% for 2-pyrrolidone and about 8% for sulfolane in PCR, representing enhancements of at least 7-10 fold in maximum tolerated concentration). L-5-2-F01-RT1 (Table 1) has even higher DNA-dependent DNA polymerase activity than L-5-2-F01, as well as significant reverse transcriptase activity. Moreover, given its solvophilicity, reverse transcription with this enzyme is possible at even higher cosolvent concentrations than the maximum concentrations compatible with PCR (FIG. 2; Table 1).

In summary, we assessed the PCR efficiency based on Cq values in non-optimized buffer in a limited number of 16 PCR cycles and even from early CSR rounds identified nineteen mutants which showed better efficiency than the wild type enzyme, in order to assess the benefits of the solvostabilizing mutations in the context of applications including RT-qPCR. We demonstrated that some of our mutants can efficiently amplify the c-Jun template—a template which cannot be amplified by Taq polymerase in the absence of BD—in the presence of up to 10% BD (FIG. 12C and Table 9). Overall, the results on GC-rich template amplification (FIGS. 5, 10, and Table 13) demonstrate that PCR using WT cannot be optimized to amplify many GC-rich genes, whereas the reported engineered polymerases are capable of amplifying templates of nearly any GC content. Regardless of the denaturation temperature used, the much greater inhibitory effect of cosolvent on WT enzyme activity limits the maximum % BD that can be used. In contrast, the engineered polymerases overcome these limitations that prohibit robust GC-rich template amplification.

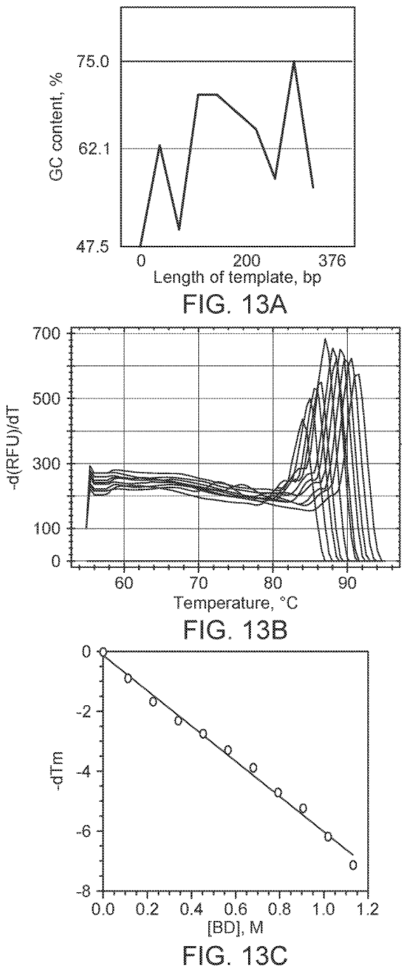

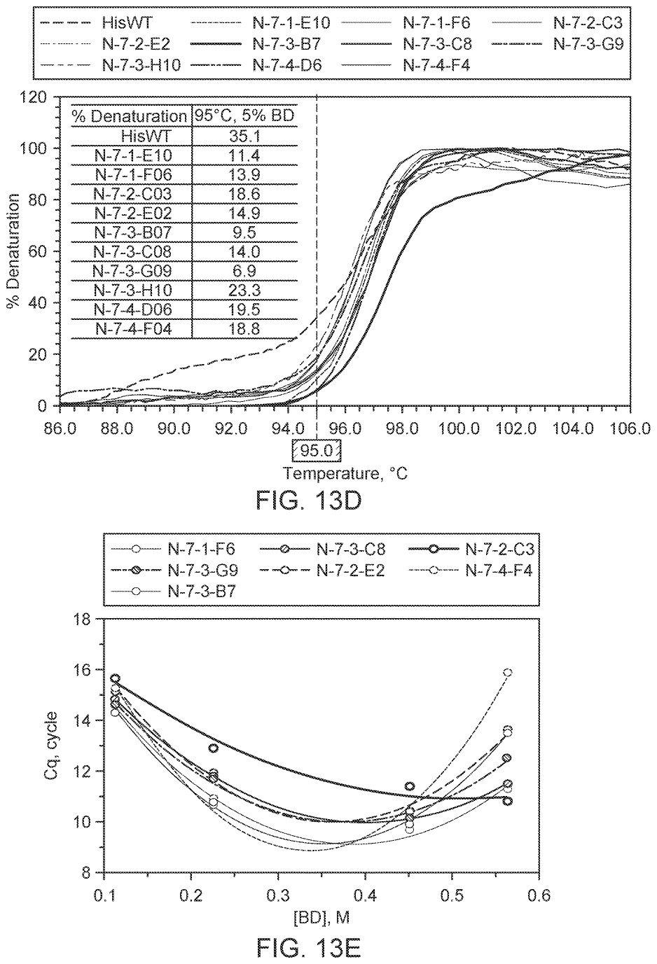

To deconstruct the net effect of BD on amplification by our polymerase variants, we first determined the reduction of DNA template Tₘ as a function of BD concentration. This analysis also has implications for RT-PCR using solvophilic RTs in the presence of the cosolvent(s). We amplified the c-Jun template. The average theoretical % GC was determined to be about 64%, but some regions were over 70% GC (FIG. 13A). We added BD ranging from 0-10% and ran melt-curve cycles. We found a linear correspondence between BD concentration and reduction in DNA melting temperature (Tₘ). The melt curve traces are shown in FIG. 13B. The slope of the plot (dTₘ/[BD]) is −5.9 K/M (FIG. 13C), which is within the range of the previously reported value for 1,4-butanediol. Furthermore, the protein melting T_{M}s of the WT and mutant polymerases respond to BD concentration negatively (Table 6) with a linear relationship and the % enzyme denatured is displayed for several mutants in 5% BD in FIG. 13D. In general, the melting temperature of the WT-Taq polymerase decreases more per unit BD concentration, compared to the mutants, consistent with the fact that the engineered polymerases resist the denaturation effect of BD. Enzyme activity data at 0 and 5% BD, demonstrate differential rates of activity loss. FIG. 13E depicts the net effect of these properties on Cq values as a function of BD concentration and the concentrations at which maximal PCR efficiency is achieved for each. The properties of the top hit clones (mutants from N series 7^{th} round, mutants from L series 5^{th} round and synthetic sequences) with the best performance in terms of specific activity, thermostability, and/or PCR efficiency in the presence of 1,4-butanediol are summarized in Table 1. Due to their improved butanediol organic solvent-tolerance and high thermal stability, the top hit clones were next systematically evaluated for their ability to improve GC-rich template amplification and to reduce GC bias in next-generation sequencing.

Nonlinear (RT-)PCR amplification dynamical models relate polymerase activity and thermostability as well as nucleic acid secondary structure and duplex melting temperatures to (RT-)PCR product yield and Cq value. The proposed model can be used to predict the Cq value as a function of cosolvent concentration, given the effects of cosolvent on each of these three properties, and thus to identify the optimal cosolvent concentration for amplification of a given template with a characterized polymerase enzyme. Furthermore, activity decline (of DNA-dependent DNA polymerases) and thermal denaturation, by cosolvent, increase the minimum extension time which in turn decreases product yield. In particular, the cosolvent concentration at which activity is extinguished or half-life becomes negligible determines the effective range of cosolvent concentrations because of the greater effect of the cosolvent on the enzyme activity at those concentrations. In order to determine the overall effect of BD on c-Jun PCR amplification, we plotted the values of Cq over a range of BD concentrations and interpreted the concentration of maximal efficiency (minimal Cq)—which depends on the DNA template—in terms of the competing effects of BD on DNA melting, polymerase activity and thermostability (FIG. 13). The measured Tₘ of c-Jun (which was chosen for illustrative purposes and not because it was the highest GC content template) reported in FIG. 13 is ^{~}92° C., which is very close to the predicted Tₘ of 90.5° C. (Table 13). For more GC-rich sequences, due to the higher template Tₘ, the optimal cosolvent concentration is higher. As shown in FIG. 13B with the measured DNA melting curves for c-Jun, and the fact that the Tₘ only reduces by <1° C./% BD (FIG. 13C), very little of the template DNA will be denatured at BD concentrations below the optimal BD concentration for WT. Since we engineered both more thermostable (Tables 5B, 6 and 7) and more active polymerases (Table 12), these enzymes show better product yield (as measured by Cq, FIG. 13E) and tolerate a wider range of solvent concentrations, which is consistent with the PCR model. BD concentrations above the optimal concentration reduced polymerase thermostability and activity to an extent that did not compensate for further marginal improvements in DNA melting for this template.

GC-rich template amplification with engineered polymerases: In addition to c-Jun, a broad set of GC-rich templates from genomic DNA (Table 13) was PCR-amplified with WT and one engineered polymerase (L-5-2-F01; Table 3), in the presence of BD. Two types of PCR cycling conditions (high and moderate denaturation temperature) were employed, with several BD concentrations. FIG. 5B shows that even under high denaturation temperature, WT is incapable of amplifying the seven GC-rich templates even in the presence of 7% BD. Interestingly, by increasing the BD concentration to 10% (FIG. 5D), the engineered polymerase variant is capable of amplifying all seven of the GC-rich templates (with some degree of nonspecificity for CD5R2 and DACT3, which have among the highest GC contents at 64% average/88% max and 79% average/~100% max, respectively, Table 13 and FIG. 11). With the engineered polymerase under these conditions, the BAIP3 template (GC %: 64% average/80% max) showed strong amplification, with only one nonspecific band, while KLF14 (GC %:72% average/90% max) showed significantly lower specificity (Table 13 and FIG. 11).

Finally, we compared PCR amplification using these polymerases under lower denaturation temperatures (FIG. 5 (E-G)), where four highly GC-rich templates were studied, including DACT3 and KLF14 and CDN1C and PO3F3 (GC contents 77% average/98% max and 78% average/93% max, respectively). Under these conditions, KLF14 was strongly amplified with high specificity using the engineered polymerase. CDN1C and P03F3 were also effectively amplified with high specificity. DACT3, containing the highest max GC content of all templates studied, could not be amplified under lower denaturation temperatures. WT can produce some amplification of only one out of four templates (KLF14) under these cycling conditions in 7% BD; for KLF14, the amplification yield was significantly less than that for the engineered polymerase under the same conditions. Thus, all GC-rich sequences studied were effectively amplified using the engineered polymerase, but almost none of them were effectively amplified using WT.

The overall results on GC-rich template amplification (FIGS. 5, 10, and Table 13) are also fully consistent with the first-principles model for PCR amplification that explains amplification yield in terms of cosolvent effects on enzyme thermostability/activity and DNA melting. This enables rational optimization of GC-rich template amplification including RT-PCR of GC-rich templates. The DNA-dependent DNA polymerase, which may or may not be the same enzyme as the reverse transcriptase, must be stable in the presence of both organic cosolvent concentrations and the denaturation temperatures used with such templates. Specifically, in FIG. 5 (high denaturation temperature), it is not possible to raise the BD concentration for the WT polymerase at this denaturation temperature to achieve a reduction in template Tₘ sufficient to amplify most of the GC-rich templates, whereas this is possible for the engineered polymerase. In this regard, note that 5% BD reduces the template Tₘ about 3-4° C. (FIG. 13C), which may be sufficient for some templates if the denaturation temperature is set to 97° C. or higher (due to the fact that only 50% of the template is denatured at the Tₘ), but the WT thermostability is negligible in 5% BD at these temperatures. In FIG. 5 (A-D) (high denaturation temperature, high % BD) and FIG. 5 (E-H) (lower denaturation temperature, high % BD), it is not possible to find a temperature/% BD combination for WT that amplifies DACT3 (or other GC-rich templates) by reducing Tₘ sufficiently without overly compromising stability at the chosen denaturation temperatures. By contrast, DACT3 was successfully amplified using the engineered polymerase in FIG. 5D because template Tₘ could be reduced by ^{~}6-7° C. by using 10% BD, which enables significant template denaturation at 98° C.—a temperature which the engineered polymerase can withstand in concentrated cosolvent. We note that while WT thermostability at 95° C. is reasonable in 5% BD (Table 5B), given that 5-7% BD reduces template Tₘ by only 4-5° C., the reduction in template sufficient Tₘ is not sufficient to amplify highly GC-rich genes like P03F3 and DACT3. Moreover, WT activity is significantly reduced at 5% BD. This analysis demonstrates that polymerase characterization can be used to predict optimal conditions to enable amplification of otherwise intractable GC-rich sequences. The principles directly extend to RT-PCR of such templates (see for example the results presented herein for the RT step with the GC-rich RNA template for the BEGAIN gene), with higher cosolvent concentrations reducing the secondary structure of structured RNA templates and also enhancing activity of the enzyme.

With respect to RT reactions, several factors besides the catalytic activity of the reverse transcriptase enzyme may play important roles in determining the effective range of such polar organic cosolvents: a) Due to primer-template destabilization by organic cosolvents, especially at higher cosolvent concentrations an initial incubation at lower temperature for cDNA primer annealing prior to high temperature reverse transcription to fully extend the primer may further increase reverse transcription product yield; b) For the higher molecular weight, potent polar organic cosolvents like BD, sulfolane, tetramethylene sulfoxide or 2-pyrrolidone at or above 20% v/v cosolvent, the melting temperature of the reverse transcriptase may begin to approach optimal extension temperatures of thermostable reverse transcriptases. Solvostabilizing mutations can broaden the window for effective high temperature catalytic activity of reverse transcriptases at these cosolvent concentrations (compare SFM4-3, 4-6 with L5-2-F01-RT1 and -RT2 in FIG. 2 as well as Table 6A). Protein stabilizing adjuvants like glycerol can also be helpful. The protein melting temperatures of four thermostable reverse transcriptases measured by nanoDSF (Table 6A) demonstrate that the enzymes not specifically engineered for solvostability and activity in organic cosolvents display significantly lower melting temperatures—especially in the presence of organic cosolvents—than those that were engineered for function in mixed aqueous-organic media. This significant reduction in stability may limit the utility of such enzymes in one enzyme RT-PCR, compared to certain solvostable reverse transcriptase enzymes; c) Finally, the Tₘ, of the RNA:DNA heteroduplex (reverse transcription product) is reduced by cosolvents. Such effects may play a role in limiting the effective range of RT activity-enhancing polar organic cosolvents.

With respect to fidelity, RTs generally have lower fidelity than DNA-dependent DNA polymerases and Taq variant RTs have been engineered to achieve fidelities close to several of the highest fidelity RTs reported to date without the need for a proofreading domain. Fidelity data on the engineered solvostable RTs (Table 11) show that they do not generally have lower fidelities than other Taq variants.

Fidelity of the mutants: Polymerases with high normalized peak area at 5% and/or 7% BD, including those which ranked most highly in the above computational and experimental activity and stability analyses, were selected for further characterization. As shown in Table 11, the fidelity of the engineered polymerases is similar to that of the wild type enzyme with the exception of SPC9, a synthetic variant which has slightly lower fidelity.

Reverse transcription within droplets, and reverse transcription and RT-PCR assays from cells enhanced by organic cosolvents: For illustration of reverse transcription within emulsion droplets, direct detection of pico-green fluorescence from emulsion droplets was applied. Droplets were generated after mixing EnzChek RT buffer with brain total mRNA and the BEGAIN primer. As shown in the upper panel of FIG. 7(L), a clear fluorescence signal could be detected by microscopy when the droplets were processed after PCR. Prior to the PCR reaction, all droplets appeared uniform, and no bright droplets were observed. As a negative control, both pre-PCR and post-PCR images of the droplets lacking brain total mRNA did not show any bright droplets, indicating the absence of DNA formation. This result suggests that fluorescence-activated cell sorting (FACS) could be used to sort the RT-PCR or RT-only products within the droplets.

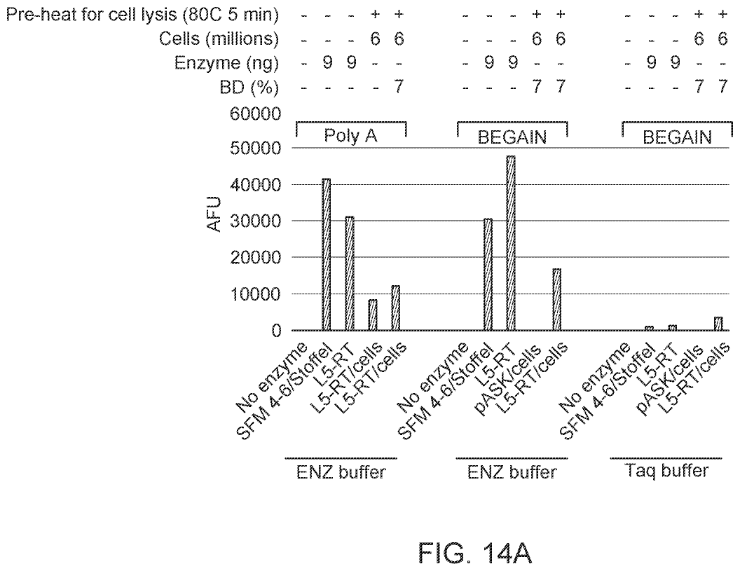

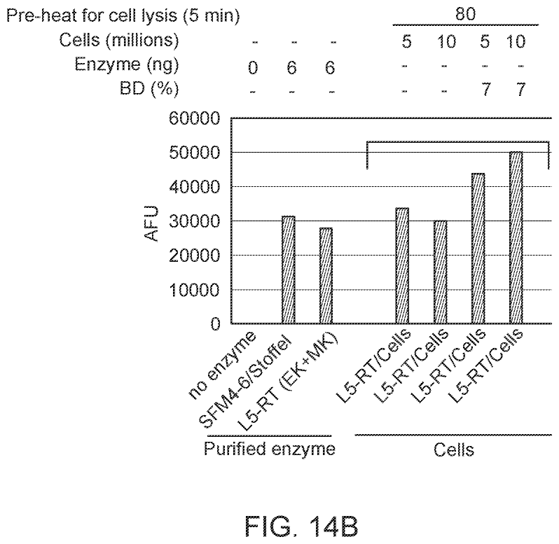

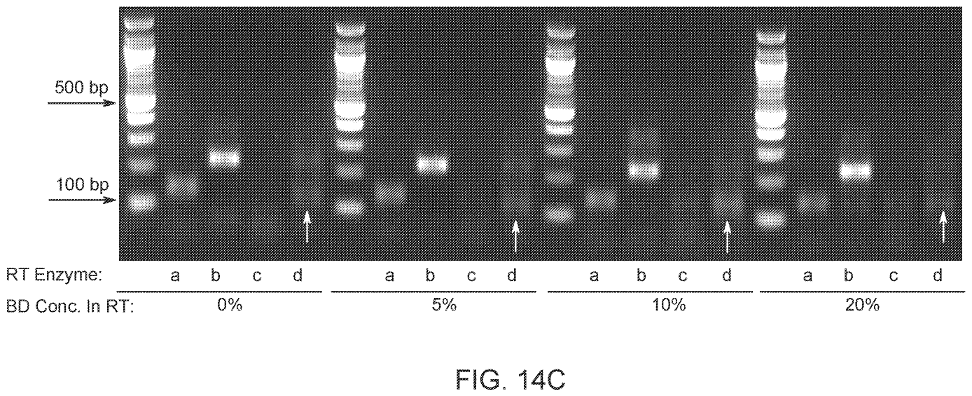

Reverse transcription and RT-PCR assays were also carried out from cells, as follows. As shown in FIG. 14, the performance of the reverse transcriptases (RTs) from induced bacterial cells carrying wild-type Taq, SFM4-6, and L-5-2-F01-RT1 was evaluated using a two-step RT-PCR assay in the presence of different concentrations of the organic cosolvent 1,4-butanediol. The results demonstrate that the activity of the L-5-2-F01-RT1 enzyme improved at higher BD concentrations, except at the highest concentration tested. This finding is consistent with the results previously reported in FIG. 2 for the purified L-5-2-F01-RT1 enzyme. As a positive control, the purified SFM4-6 RT generated the expected DNA band across all BD conditions. However, its activity decreased at high BD concentrations, as anticipated. While a non-specific band was observed in the negative control lanes containing the wild-type Taq cells, no KRAS-specific band was detected, confirming the validity of the negative control. The cells expressing the SFM4-6 RT produced faint bands, indicating that its RT activity was weaker than that of the L-5-2-F01-RT1 enzyme under these experimental conditions.

In summary, in addition to making the unanticipated discovery that highly thermostable reverse transcriptases are generally solvophilic, i.e. upregulated by polar organic cosolvents, we used droplet-based directed evolution to engineer solvostable polymerases even more suitable for RNA-dependent DNA polymerization (RT) and RT-PCR applications. These cosolvent-resistant engineered polymerases arguably solve the longstanding sequence bias problem of nucleic acid polymerization. These compositions and methods, including cosolvents and polymerases, are expected to work not only in standard RT and RT-PCR but in any related protocols such as NGS/RNA-seq, droplet digital RT-PCR (ddRT-PCR), and infectious pathogen RNA detection without sample preparation. Our top mutants, like L-5-2-F01-RT1, exhibited exceptional reverse transcriptase activity at high temperatures in the presence of organic cosolvents, significantly higher than in aqueous media. Moreover, with such solvent-tolerant polymerases, a broader range of cosolvent concentrations and a broader spectrum of cosolvents may be compatible with nucleic acid polymerization and amplification. In particular, due to the enhancement of reverse transcriptase catalytic activity by polar organic cosolvents, it is expected that higher molecular weight cosolvents (in addition to higher concentrations of the same cosolvents, as shown) are compatible with reverse transcription compared to DNA-dependent DNA polymerization and PCR, even for thermostable RTs that are not engineered for performance in aqueous-organic media.

A non-limiting listing of thermostable reverse transcriptases having properties described herein is provided in Table 15 below.

### Methods

Bacterial strains, plasmid, and chemicals: Electrocompetent Escherichia coli TG1 expression hosts were purchased from Lucigen (WI, USA). The codon-optimized WT-Taq gene was synthesized at Genscript (NJ, USA). The gene was cloned in pASK-IBA5C vector (IBA Lifesciences, Germany) between XbaI and SalI restriction sites to create the plasmid pASK-Taq. Restriction enzymes, Q5, Vent polymerase, T4 DNA ligase, Calf Intestinal Phosphatase (CIP), and M13 single stranded DNA were procured from New England Biolabs Inc (MA, USA). Diversify PCR random mutagenesis kits were purchased from Takara Bio USA Inc. (CA, USA). All primers were synthesized at Integrated DNA Technologies (Iowa, USA). Primer sequences used in this study are given in Table 14. DNA sequencing was carried out at GENEWIZ (NJ, USA). Chloramphenicol and anhydrotetracycline were purchased from Sigma-Aldrich (MO, USA). All other chemicals were of highest purity available, purchased either from Sigma-Aldrich or VWR International.
Reverse Transcriptase activity (RT) assay: We synthetically generated three mutant clones (among others) by introducing three mutations E742K+M747K and D732N in two of our best performing mutants. These clones were: 1) L-5-2-F01+E742K+M747K (L-5-2-F01-RT1), 2) N-7-3-B07+E742K+M747K (N-7-3-B07-RT), and 3) L-5-2-F01+D732N (L-5-2-F01-RT2). The enzymes of interest were tested for RT activity using EnzChek Reverse Transcriptase Assay Kit (E-22064) in the presence of varying concentrations of cosolvents (BD, 2-pyrrolidone, sulfolane and tetramethylene sulfoxide). WT-Taq was used as negative control. A Stoffel fragment mutant of Taq polymerase (SFM 4-6) reported previously to have RT activity and ProtoScript II RT were used as positive controls. Equal activities of the enzymes were applied in the master mixture (total volume 30 μl) and the mixture was incubated at 55° C., and the same quantities of each enzyme were also incubated at 68° C. and 76° C., for 30 min. The reactions were terminated by addition of 2 μl of 200 mM EDTA. 68 μl of the PicoGreen solution (as recommended by the kit for solution preparation) was added into the solution and incubated for 5 min at room temperature. The RT activity was measured as intensity of fluorescence using TeCan (Infinite 200Pro) microplate reader with excitation/emission wavelengths 480 nm and 520 nm respectively. The standard curve was measured at 37° C. using a commercially available reverse transcriptase (ProtoScript II RT, NEB) to calculate the relative activity. The data were collected and analyzed using GraphPad Prism 7.
RT-qPCR assay: RT-qPCR efficiencies of our two best mutants L-5-2-F01+E742K+M747K (L-5-2-F01-RT1) and N-7-3-B07+E742K+M747K (N-7-3-B07-RT) were tested in absence and presence of BD (7%) and compared with the SFM4-6.

In step 1, 0.2 μg of a synthesized RNA of human BEGAIN gene fragment (100 base) with 75% GC content (CCUGCGGGCCAAGCCGGGGACCGCCCGGCUCCCCGGGGAGGACAUGAGGGGCCAGUGGCGUCC CCUGAGCGUGGAGGACAUCGGCGCCUACUCCUACCCC) (SEQ ID NO: 146) was incubated with 0.5 μg of BEGAIN RT-REV at 80° C. for 10 min in 100 uL H₂O (RNase and Dnase free). In step 2, RT reaction mixture (a total volume of 20 μl) was prepared using 1×Taq buffer (−Mg), 0.25 mM dNTP, 3.5 mM MgCl₂, RNase inhibitor (NEB), 1×SYBR safe, BD (0% or 7%), template RNA-primer mix (10 μl) from step 1 and 30 ng of each enzyme. RT reaction was carried out at 55° C. or 68° C. for an hour followed by deactivation of the above enzymes at 98.3° C. for 1 min+95° C. for 6 min. In step 3, qPCR reaction mixture (20 μl) was prepared using 1×Taq buffer (−Mg), 0.25 mM dNTP, 3.5 mM MgCl₂, 1×SYBR safe, RT reaction mixture from step 2 (1 μl), BEGAIN RT-FWD, BD (7%) and L-5-2-F01 enzyme (3.55 ng/0.625U). The Bio-Rad CFX96™ Real-Time PCR Detection System was used to carry out the qPCRs using 1 min at 95° C. followed by 40 cycles of 30 s at 95° C., 30 sec at 57° C., and 40 sec at 72° C. The Cq values were determined to assess the efficiency of RT-qPCR. The melt curves of the PCR samples were run from 55° C. to 95° C.

Library construction: We generated error-prone PCR (epPCR) Taq library (also termed generation 1, pre-1^{st} CSR round) using codon optimized WT-Taq DNA sequence as a template. We define “generation” in terms of how many times diversity was introduced in the original epPCR library—e.g., when WT-Taq sequence was diversified by random mutagenesis first time, it is called “generation 1”—whereas the number “round” denotes the number of times the library has gone through CSR—e.g., post-1^{st} CSR round means that the library was selected after one round of CSR. We used the commercially available Diversify epPCR kit (Takara). We performed epPCR using primer pair CSR-Select-F and AU-Gen-Taq-R (Table 14) to generate 3-4 mutations per sequence. To assess the quality of the library, we subjected the post-epPCR library to NGS and confirmed with limited number of Sanger sequencings. Both of our approaches confirmed that the library is of good quality and that we generated an average 3-4 mutations per sequence. Similar mutational load has been used in existing literature on polymerase evolution. The PCR products were DpnI digested followed by column purification using Qiagen's PCR purification kit. The purified products were digested by XbaI and SalI and introduced into the pASK-IBA5C vector. The ligated products were electroporated into E. coli TG1 cells. After an hour of recovery, 5 μL cells were serially diluted to spread on the LB-chloramphenicol (50 μg/ml) plates to assess the library size. The remaining cultures were re-inoculated into 20 ml LB media supplemented with chloramphenicol in 50 ml conical flask overnight at 37° C. and shaking at 250 RPM to generate N-epPCR expresser cells.
We first established that during CSR, the water-in-oil compartments are separate, non-communicating, and unaffected by heat and organic solvent. Our data show that emulsion droplets are stable after 30 cycles of thermal cycling in up to 10% BD. The efficiency of our CSR procedure was assessed through a selection experiment wherein we mixed 90% cells expressing WT polymerase and 10% cells expressing a thermostable mutant T8, and following CSR recovered several fold more thermostable clones than WT clones.
Generation of expresser cells and compartmentalized self-replication: To generate expresser cells, post-transformation libraries were inoculated in 20 ml of LB-chloramphenicol in 50 ml conical flask overnight at 37° C. and shaking at 250 RPM. Following outgrowth, N-epPCR expresser cells (1%) were inoculated into 50 ml of LB-chloramphenicol. The cells were induced by anhydrotetracycline (300 ng/ml) to express the Taq polymerase once the OD₆₀₀ reached between 0.4-0.5. After four hours, the cells were harvested by centrifugation, washed, and resuspended in 1×Taq buffer (10 mM Tris-HCl pH 8.5, 50 mM KCl, 1.5 mM MgCl₂, and 0.1% Triton X-100). The CSR was performed as described by to select for thermostable and BD resistant mutants in 5% BD. The emulsions were pre-incubated at 95° C. for 6 min, followed by CSR PCR, 25 cycles at 94° C. for 1 min, 55° C. for 1 min, and 72° C. for 5 min. To add further diversity, the top hit clones were randomly recombined by StEP PCR. The resulting library (generation 2, pre-1^{st} CSR round) was subjected to higher selection pressure. The CSR-PCR was performed in the presence of 7% BD. The CSR-PCR cycle used was as follows: 98.3° C. for 1 min then 95° C. for 6 min, followed by 25 cycles at 94° C. for 1 min, 55° C. for 1 min, and 72° C. for 5 min.

The library series generated via error-prone PCR are referred to as the N-series (or epPCR libraries) for short. The initial error-prone PCR-generated library is labeled N-epPCR. Downstream CSR-treated libraries are labeled referring to the number of CSR rounds applied to this library. CSR-treated libraries are referred to N-1^{st}, N-2^{nd}, N-3^{rd}, N-4^{th}, N-5^{th}, N-6^{th}, and N-7^{th} corresponding to the total 7 rounds of CSR treatment applied to this library. We used staggered extension process (StEP-PCR) to generate diversity in top selected clones. We used high fidelity Vent polymerase for the StEP recombination as described previously. We isolated plasmids from top ranked clones after CSR, and restriction digested with XbaI and SalI to generate the StEP template. The reaction consisted of an equimolar mixture of each fragment (total 0.15 pmoles) supplemented with 25 pmoles of primers CSR-select-F and AU-Gen-Taq-R, 250 μM dNTP, and 1.5 units Vent polymerase in 1×Thermopol buffer. The PCR extension protocol was as follows: initial denaturation at 95° C. for 5 min; 150 cycles of 95° C. for 1 sec; 55° C. for 5 sec; 72° C. for 2 sec; and final extension at 72° C. for 2.5 min. The PCR product was treated with DpnI, precipitated with sodium acetate, and digested with XbaI and SalI to clone in to the pASK vector for the next round of the CSR. Even though we used high-fidelity Vent polymerase for StEP PCR, we noted extra mutations, possibly due to the abbreviated nature of PCR in StEP PCR. The library series generated via StEP shuffling are referred to the L-series, StEP, or shuffled libraries within this document. The initial StEP shuffling-generated library is labeled L-StEP. Downstream CSR-treated libraries are labeled referring to the number of CSR rounds applied to this library. CSR treated libraries are referred to L-1^{st}, L-2^{nd}, L-3^{rd}, L-4^{th}, and L-5^{th} corresponding to the total 5 rounds of CSR treatment applied to this library series.

Library enrichment and next-generation sequencing: To enrich and identify best-performing clones, we subjected the epPCR library series to seven consecutive rounds of CSR in the presence of 5% BD. After each cycle of the CSR enrichment rounds, the PCR product was re-amplified, cloned and then transformed in E. coli TG1 cells as mentioned above. After the 1^{st}, 3^{rd}, 5^{th} and 7^{th} rounds of enrichment CSR for the epPCR library, a fraction of the library was subjected to a screening protocol followed by Sanger sequencing (^{~}1500 clones screened for rounds 1 and 7 and ^{~}250 clones screened for rounds 3 and 5). A similar screening protocol was carried out for L series (^{~}1500 clones screened for rounds 1 and 5). Overall, 0.37% of all screened clones from the first round of CSR on the epPCR library performed better than the WT whereas this ratio was 0.13% and 40% for clones from the first round of CSR on the shuffled library and synthesized clones (see below), respectively—demonstrating the effectiveness of synthetic recombination. We isolated the plasmids after each enrichment round for NGS analysis. The library plasmids were digested with XbaI and HindIII, generating a slightly bigger fragment than the Taq ORF. The Taq gene (^{~}2.5 kbp) was arbitrarily divided into six fragments (Fragments 1-6 and the amplicon size ranged from 450 bp-468 bp) to make it compatible with Illumina sequencing platform. We used high-fidelity Q5 polymerase to amplify these fragments using six sets of overlapping primers, NGSR1_FWD and NGSR1_REV; NGSR2_FWD and NGSR2_REV; NGSR3_FWD and NGSR3_REV; NGSR4_FWD and NGSR4_REV; NGSR5_FWD and NGSR5_REV; NGSR6_FWD and NGSR6_REV (Table 14). The PCR products were gel purified and then subjected to the Illumina NGS protocol at GENEWIZ. Forward and reverse sequence reads were merged and filtered with a sequence quality score cutoff of 33, using the PEAR assembler. Filtered and assembled sequences were then aligned to the reference gene sequence (WT-Taq gene) using sequence matcher and pairwise2 modules of the Biopython software suite. We used alignment parameters 2, −1, −35, −0.1, for identical, non-identical, gap opening, and gap extending, respectively, for both alignment tools. Resulting unique merged sequences and alignment scores were recorded. Non-target, large frameshifted, and truncated data were removed based on an alignment score as a sequence length cutoff of amplicon length (−6 to +1 bp). Final sequences were translated to their corresponding in-frame amino acid sequences by the Biopython translate module; sequence changes were recorded and logged in a tabular format. Frequency of any mutation found in a read within a region was calculated with Equation 1: Dolomite

$$Frequency of Mutation = \frac{Summed Reads of Sequences that Contain Mutation}{Summed Total Region Reads} * 100 \qquad ( 1 )$$

Fold enrichment for any one amino acid position or individual mutation was calculated with Equation 2:

$$Fold Enrichment = \frac{Frequency of Mutation ( POST )}{Frequency of Mutation ( INITIAL )} \qquad ( 2 )$$

Although we see some loss in the frequency during the CSR rounds especially in the 6^{th} round of CSR, possibly due to total number of reads acquired, we observed a general trend that the most fit mutants are highly represented in the final library. We further observed that the enriched positions do not cluster in a hotspot but are distributed in both the exonuclease and polymerase domains.

Using one round of CSR followed by screening thousands of clones, we were unable to get genotype redundancy in Sanger sequencing. In early rounds of CSR, genotype redundancy and identification of highly performing mutants requires screening of a very large number of clones.

FACS of droplets was also used as an alternative method to enrich for high-fitness library variants.

Synthesis of mutant clones: To generate further diversity, we generated synthetic clones by recombining the top hits from the epPCR and StEP libraries. In general, we restricted the number of mutations per sequence to 6-10 to avoid mutational overload. A total of nine clones (Synthesized polymerase clones; SPC1-9) were synthesized at Genscript. SPC1-9 were based on mutations identified from wet lab screening after one round of CSR.
Microfluidic droplet preparation for CSR and polymerase screening by FACS: We noted one of the challenges with manual droplet emulsion preparation is the polydispersity. To circumvent polydispersity and further improve the selection of hit clones, we employed a computer-controlled microfluidics device to prepare a) water-in-oil (w/o) primary emulsion (PE) for use in CSR and b) water-oil-water (w/o/w) double emulsion (DE) compatible with FACS sorting, to sort mixtures of the best performing engineered polymerase clones obtained from CSR selection, based on their PCR performance against arbitrary templates—and thus validate their performance with a secondary screen using monodisperse droplets.
We prepared expresser cells as described above. We used FluoSurf (2% in HFE7500;) as the oil phase. We used Dolomite's pEncapsulator system and 30 μm fluorophilic chip to generate a 20 μm, monodispersed, primary emulsion (PE) following the manufacturer's protocol. For double emulsion preparation, we loaded one channel of the reservoir chip with PE, while the other channel was loaded with FluoSurf as spacer fluid. All the three P-pumps were loaded with outer carrier phase driving the PE and spacer fluid into the 30 μm hydrophilic chip. Typical flow rates for double emulsion preparation were as follows: 0.8 ul/min for P1 and P2-pumps whereas 8 ul/min for P3-pump, generating 30 μm DE. Droplet generation was monitored by an in-built, high-speed camera.

For CSR experiments (application a) above), this single emulsion was used directly in terminal rounds of CSR experiments as described above. Three P-pumps were connected to the reservoir chip driving oil phase (P3-pump) and two (P1 and P2 pumps) to push the samples into the sample chip. Typical flow rates, for PE generation, were 4 μL/min for P1 and P2 pumps whereas it was fixed at 36 μL/min for P3-pump.

To perform FACS-based experiments (application b) above) for the purpose of a secondary screen of polymerase performance, we mixed 90% WT polymerase expressing cells with 10% engineered polymerase-expressing cells. The mixed population of cells was used to generate the water-in-oil primary emulsion. The primary emulsions were subjected to PCR. We employed the following PCR cycles—95° C. for 6 minutes followed by 25 cycles of 95° C. for 1 min, 55° C. for 1 min, 72° C. for 5 min. To prepare the double emulsion, we used a 30 μm hydrophilic chip. We used an outer carrier phase containing 1% Tween-20, 2% Pluronix F68 in 1×PBS. Double emulsion encapsulation of PEs containing single bacterial cells was separately verified using GFP expressing cells. Double emulsion sorting was performed at Flowmetric Inc. (Doylestown, PA). We used FACSAria II sorter (Becton Dickinson Biosciences) to sort double emulsions. Prior to sorting, the double emulsions were diluted to 1:5 using FACS diluent (1% Tween-20 in PBS) and then stained with SYBR Green I. Using both positive and negative controls, the FACS machine was calibrated on forward scatter, FSC. The instrument was further calibrated to identify negative and positive droplets using negative and positive controls, respectively. Droplets were first gated using FSC-H and FSC-A followed by Alexa Fluor 488 to detect DNA-SYBR complexes. Sorting data was analyzed using FACSDiva software version 6.1.3. Based on SYBR intensities, the samples were collected as SYBR^{HIGH}, SYBR^{MEDIUM} and SYBR^{LOW} for downstream processing. Although the SYBR^{HIGH} population represents most of the droplets, SYBR^{MEDIUM} contributes to the sorted population possibly because SYBR Green I binds to bacterial chromosomal DNA. To isolate DNA from sorted samples, the excess sheath fluid was removed from SYBR^{HIGH} sample then mixed with 2× volume of 1H, 1H, 2H, 2H-Perfluoro-1-octanol (PFO) to break the droplets, followed by Sanger sequencing to quantify enrichment of each clone.

Real-time qPCR screening assay: A SYBR Green I assay based on real-time qPCR was used to screen the transformants obtained following CSR selection. Transformed colonies were picked and inoculated into a 96-deep well culture plate containing 500 μL LB-chloramphenicol medium. Cells were grown and induced by anhydrotetracycline (300 ng/ml) once the OD₆₀₀ reached between 0.4-0.5. Then the cells were harvested and resuspended in 200 μL of 1×Taq buffer (10 mM Tris-HCl, pH 8.0, 50 mM KCl, 1.5 mM MgCl₂, 0.1% Triton X-100). The PCR mix contained 10 μL of cell suspension and 40 μl master mix 1,4-Butanediol (5% (v/v), 0.25 mm dNTP, 1 mg/mL BSA, 3.5 mM MgCl₂, 0.5 μM of primers-Taq Q1, Taq Q2 (Table 14), and 0.5×SYBR Green I. We used Bio-Rad CFX96™ Real-Time PCR Detection System to carryout PCR using the following program—6 min at 95° C. followed by 16 cycles of 30 sec at 94° C., 30 sec at 57.8° C., and 30 sec at 72° C. Melting curve analysis was performed between 55 and 95° C. at 0.1° C./s melt rate. Melting peaks were visualized by plotting the relative fluorescent value (RFU) of the 1^{st} derivative against the temperature. We calculated the peak areas using GraphPad Prism software and normalized the peak area to cell number to rank the clones.

Hit clones from screening were denoted using the following notation: Library #-Round #-Plate #-Well #. For example, N-7-1-E10 refers to a clone isolated from the epPCR library (N) after 7 CSR rounds on plate 1 in well E10; whereas L-1-14-H10 refers to a clone isolated from the shuffling library (L) after 1 CSR round on plate 14 in well H10.

Protein purification: We introduced His-tag to the top ranked clones by PCR using Q5 site-directed mutagenesis kit (NEB). The primer (His-F and His-R) sequences are listed in Table 14. Following transformation, we confirmed the His-tag by DNA sequencing. Single colonies expressing either WT polymerase or mutant derivatives were grown overnight at 37° C. in 5 ml LB-chloramphenicol. The overnight grown cultures were re-inoculated into 200 ml of LB-chloramphenicol. The protein expression was induced by anhydrotetracycline (300 ng/ml) once the OD₆₀₀ reached between 0.4-0.5. The cells were harvested by centrifugation after 4 hours, washed once, and resuspended in 2.5 ml wash buffer (50 mM Tris-HCl, pH 7.9, 50 mM dextrose, 1 mM EDTA, 1 mM PMSF). The cell suspensions were subjected to two cycles of freeze-thaw. The partially lysed cells were incubated with 1 mg/ml lysozyme at room temperature for 15 min. Following incubation, an equal volume of lysis buffer (10 mM Tris-HCl, pH 7.9, 50 mM KCl, 1 mM EDTA, 1 mM DTT, 1 mM PMSF, 0.5% Tween-20, 0.5% Nonidet P40) was added; the sample was kept on ice for 30 min. The crude lysates were then incubated at 75° C. for 30 min followed by centrifugation to collect the supernatant. The nucleic acids were precipitated by streptomycin sulfate. The solution was centrifuged, and the supernatant was loaded onto an IMAC column. The column was washed with equilibration buffer (10 mM Tris-HCl, pH 7.9, 50 mM KCl, 20 mM imidazole), and eluted with 10 mM Tris-HCl, pH 7.9, 50 mM KCl, 300 mM imidazole. The proteins were dialyzed against dialysis buffer containing 20 mM Tris-HCl, pH 8.0, 1 mM DTT, 0.1 mM EDTA, 100 mM KCl, 0.5% NP40, 0.5% Tween-20 and 50% glycerol. The polymerases were quantified using Bio-Rad's DC protein assay. We resolved purified proteins on SDS-PAGE to assess the purity of the preparations. To produce non-His-tag proteins, we introduced a protease cleavable His-tag at the N-terminus of the WT-Taq using primer set 6His-TEV-R and 6His-TEV-Ser-F (Table 14). After PCR using Q5 DNA polymerase, we transformed, and then sequenced to verify the cleavable His-tag. Once confirmed, the protein was expressed as described above. The cleavable, purified protein was subjected to the TEV protease (NEB). The cleaved His-tag was removed by loading on to IMAC column, collecting the flow-through followed by dialysis. We confirmed the His-tag removal by InVision His-Tag In-Gel Stain (Invitrogen).
Nucleotide incorporation and denaturation kinetics assays: Organic cosolvents affect enzyme activity and thermostability via different mechanisms. To determine the activity of the purified enzymes, we used EvaGreen-based fluorescence assay. We used a 59 nucleotide long self-annealing template-primer (SATP). Previously, SATP has been used for DNA binding and activity assays. The SATP used in this study has 25 nucleotide overhangs for primer extension (Table 14). Briefly, 0.2 ng purified WT and its mutant derivatives were incubated with 100 nM SATP, 3 mM MgCl₂, 250 μM dNTPs, 1×EvaGreen, and 0.5 μg/μL BSA in 1×Taq buffer. The primer extension reactions were carried out at 72° C. We used commercially available Taq DNA polymerase (Invitrogen) to calculate the relative activity.

Half-lives of the enzymes were determined as described previously, except that we used EvaGreen based assay and utilized SATP to measure the remaining activity as described above. We used 10 mU (in 2 μL) of either wild type or mutant enzymes, pre-incubated in 1×Taq buffer at either at 95° C. or 97.5° C. for a) 0, 1, 3, 5, 10, 20, 40, 60 min in the presence of 5% BD, and b) 0, 2, 5, 10, 20, 40, 60, 90 min in the absence of BD. Heat treated samples were kept on ice until the reaction was started by adding 18 μL of substrate mix containing 3 mM MgCl₂, 100 nM SATP, 250 μM of each the dNTPs, and 1× EvaGreen in 1× Taq buffer. After determining the remaining activity, the half-life (t_{1/2}) was calculated by plotting the percent activity remaining versus heat exposure time at specific temperature.

Since it was suggested that the N-terminal His-tag reduces the thermostability of the Taq polymerase by 5-fold, both His-tagged and non His-tagged WT were tested for t_{1/2}. The half-life of His-tagged and non His-tagged WT was 3.7 and 3.6 min respectively at 97.5° C. when the protein alone was exposed to heat (method 1). The t_{1/2} of the His-tagged and non His-tagged WT was 3.9 and 4.2 min, respectively when ternary complex was heat treated (method 2). Based on our data Table 5A), we concluded that the differences between the t_{1/2} of the two proteins are insignificant. We found that the t_{1/2} of the WT in absence of organic solvent is about 41 min at 95° C. (Table 5B) which is close to the values reported previously.

In another variation of the assay we ensured that the enzyme is in a ternary complex [E-TP-dNTP]prior to the heat treatment. 10 mU of enzymes were pre-incubated in a reaction mix containing 3 mM MgCl₂, 100 nM SATP, 250 μM each of three dNTPs (dTTP, dGTP, and dCTP) in 1× Taq buffer (18 μL) on ice followed by heat treatment protocol as described above. After heat exposure for a specific time, samples were held on ice then incubated with 0.5 μL of 40×EvaGreen for 15 min. In the SATP sequence, the first three incoming nucleotides are dGTP followed by dATP. It is conceivable that the enzyme will form an active ternary complex when incubated with correct incoming dNTP. This is an assay we used previously to assess the stability of the polymerase ternary complex. The polymerase reaction was started by adding 1.5 μL of mix containing 250 μM dATP along with 0.5 μg/μL BSA at 72° C. followed by EvaGreen fluorescence quantification.

Thermal unfolding analysis: The thermal unfolding experiments of wild type polymerases as well as the variants (5 μM, in 20 mM Tris-HCl pH 8.0, 1 mM DTT, 0.1 mM EDTA, 100 mM KCl, and 5% glycerol) were performed using nano-scale Differential Scanning Fluorimetry (nanoDSF) on a Prometheus NT.48 instrument, with a high-temperature package and back-scatter optics, which allowed analysis of thermal unfolding and aggregation up to 110° C. Thermal denaturation of each protein was determined in triplicate by measuring changes in fluorescence at 330 and 350 nm over varying temperature, from 30° C. to 110° C., with a heating speed of 1° C./min and with a 10% sensitivity setting (fluorescence excitation power). These measurements were completed in the presence of 5% BD. Experiments were performed, in triplicate, at 2Bind GmbH (https://2bind.com, Regensburg, Germany). The ratio of 350/330 nm and scattering data were analyzed using the PR. Stability Analysis software (v. 1.1, Nanotemper Technologies, Munich Germany).

### Computational Prediction of Mutational Effects on Protein Stability and Structure:

The mutations in screened clones were modelled in silico using in-house written python scripts and minimized by PyRosetta4 toolkit using default parameters. The closest centroid distances of all-atoms and sidechain atoms of mutated residues to DNA or RNA were calculated to find mutations in screened clones that are in close proximity to the DNA or RNA ligand. The initial threshold centroid distance-cutoff was taken as 10 angstroms. Mutation residues were selected for binding affinity evaluation based on proximity to DNA or RNA and the nature of amino acid change to further apply stringency on the selection criteria and to take into account the contribution of electrostatic interactions, if any, on DNA or RNA binding affinity. Selected mutation residues were modelled in silico. To perform MM-GBSA-based binding affinity evaluation, an all-atom molecular dynamics (MD) simulation was set-up using AmberTools22 package. The DOC residue was replaced with DC in the reference and mutated complexes along with removal of ZN and EDO molecules. The reference and mutated model complexes were loaded in tleap, solvated using TIP3P water model, and charges were neutralized followed by generation of parameter files (prmtop, inpcrd). An MD simulation was performed as a three-stage process—1) minimization, 2) heating (NVT) and 3) production run (NPT). The minimization was performed for a total of 20,000 steps with the first 1000 steps of steepest descent followed by conjugate gradient algorithm for the remaining steps, within which the total energy of the complex was observed to become stable. The heating run was performed for 20,000 steps, with gradual heating from OK to 300K in first 8000 steps and continuing the remaining steps at 300K with the following parameters—dt=0.0005, ntf=1, ntc=1, ntb=1, ntt=3, gamma_In=2.0. The production run was performed for 20,000 steps with the following parameters—dt=0.0005, ntf=1, ntc=1, temp0=300K, ntb=2, ntp=1, ntt=3, gamma_In=2.0. The total simulation time accounted for 20 ps. After the production run, MM-GBSA analysis was done on 101 frames of the production run (start frame=10, end frame=110) using MMPBSA.py (of AmberTools22) and the binding affinity energy in terms of Δ[complex—(receptor+ligand)] was retrieved from the MMPBSA.py output file for further analysis.

To assess the effect of mutations on the stability of hit clones, we calculated Gibbs folding free energy change (DDG) as an indicator of protein stability. We used freely available computational programs, FoldX v4.0,1 and MAESTROweb. The output is presented in kJ/mol. Since full length Taq ternary complex (E-DNA-dNTP) is unavailable, we first modelled Taq ternary complex to include 5′-3′ exonuclease domain using MODELLER recommended DOPE-score profile and PROCHECK. We followed MAESTROweb execution protocols to calculate the free energy change, whereas to calculate DDG for FoldX, we used the following formula:

$$ΔΔ {G}_{Mutant} ( FoldX ) = Δ {G}_{Mutant / Mutation combination} - Δ {G}_{WT - Taq Ternary complex} \qquad ( 3 )$$

GC bias evaluation using NGS: To evaluate the mutant enzymes' performance in the presence of BD (1,4-butanediol) in reducing GC bias, we used them for NGS library preparation of DNA templates with different GC contents. Two different DNA inputs were used in the NGS library preparation: a) using plasmid DNA—five high GC templates (c-Jun 63%, BEGAIN 71.3%, DACT3 79.2%, P03F3 77.7%, and BAIP3 64.4%) were cloned into PUC18 plasmid and 5 ng of each prepared construct were pooled. The five templates were coamplified with 7.25 U of either WT, L-5-2-F01 or N-7-3-B07 mutant under the same conditions. 4% BD was used for WT enzyme and 10% BD was used for the mutants. The reaction mixture contained 0.75 mM dNTP, 1 mg/mL BSA, 3.5 mM MgCl₂, and 0.5 μM of each primer. The following PCR programs were used: 98.3° C. for 1 min and 95° C. for 6 min, 25 cycles of 94° C. for 30 sec, 57.8° C. for 30 sec, 72° C. for 50 sec. The PCR products were run on 1% agarose gel and purified using Qiagen DNA Gel Extraction and purification Kit. Purified PCR products were sent to GENEWIZ for sequencing and library preparation as described above. The Fastq raw sequencing data from GENEWIZ on Illumina platform were aligned to the targeted templates and read frequencies were determined for each template; b) using genomic DNA—The mutant enzymes N-7-3-B07 and N-7-3-C08 and WT were tested in NGS library preparation using 150 ng genomic DNA in absence and presence of 5% BD. Three high GC templates (B3GT6 72%, CDN1C 78%, EGFR 60% and one low GC template (KRAS 40%) were coamplified using gene-specific PCR primers under the same conditions. The following PCR cycles were used: 98° C. for 3 min followed by 30 cycles of 30 sec at 95° C., 30 sec at 58.5° C., and 30 sec at 72° C. The PCR products were purified with Monarch DNA Gel Extraction Kit (New England BioLabs) and eluted with 10 μL elution buffer. The purified products were mixed and prepared with the Nextera XT library prep kit (Illumina) according to manufacturer's instructions. The indexed libraries were subsequently purified with Illumina Purification Beads included in Nextera XT library prep kit (Illumina), and quantified using the Qubit High Sensitivity dsDNA Assay (Thermo Fisher Scientific, Waltham, MA). The average fragment size, defined as insert length plus adapter length, for each sample was calculated prior to pooling. The pooled libraries were sequenced on an Illumina iSeq100 using a 2×150 bp paired-end sequencing protocol. The raw sequencing data were demultiplexed and converted to Fastq files by iSeq100 Local Run Manager DNA Enrichment Analysis Module v2.0.1.5. The reads were then aligned to human genome assembly 37/hg19 reference sequence by BWA-MEM.
qPCR efficiency and amplification of GC-rich templates: To confirm the screening rank obtained from library screening and characterize the ability of engineered polymerases to amplify GC-rich templates, we used purified enzymes and performed the qPCR assay. The PCR mix contained 1.25 U of each enzyme in the presence of different concentrations of cosolvents (1,4-Butanediol, or Pyrrolidone or Sulfolane), and 5 ng of Taq or GC-rich template. The reaction mixture contained, 0.25 mM dNTP, 1 mg/mL BSA, 3.5 mM MgCl₂, 0.5 μM of each primer—(Q1 and Q2 for Taq template; primers listed in Table 14 for GC-rich templates), and 0.5×SYBR Green I. We used Bio-Rad CFX96™ Real-Time PCR Detection System to carry out PCR using either 6 min at 95° C. followed by 16 cycles of 30 sec at 94° C., 30 sec at 57.8° C., and 30 sec at 72° C. or 98.3° C. for 1 min followed by 6 min at 95° C. followed by 16 cycles of 30 sec at 94° C., 30 sec at 57.8° C., and 30 sec at 72° C. We determined the Cq values to assess the efficiency of the polymerases.

FIG. 12: (A,B) The following PCR cycles were used: 95° C. for 6 min followed by 16 cycles of 30 sec at 94° C., 30 sec at 57.8° C., and 30 sec at 72° C.; (C) 98.3° C. for 1 min, 95° C. for 6 min followed by 16 cycles of 30 sec at 94° C., 30 sec at 57.8° C., and 30 sec at 72° C.; (D) The following PCR cycles were used: 98.3° C. for 1 min+95° C. for 6 min followed by 20 cycles of 30 sec at 94° C., 30 sec at 57.8° C., and 50 sec at 72° C. (E) Each enzyme (5U) was tested under identical conditions. The following PCR cycles were used: 95° C. for 6 min followed by 30 cycles of 30 sec at 94° C., 30 sec at 59° C., and 30 sec at 72° C.

FIG. 5: High denaturation temperature (98.3° C. for 1 min+95° C. for 6 min followed by 25 cycles of 94° C. for 30 sec, 57° C. for 30 sec, 72° C. for 50 sec). Moderate denaturation temperature (94° C. for 2 min followed by 30 cycles of 95° C. for 30 sec, 57° C. for 30 sec, 72° C. for 50 sec). A final extension was done at 72° C. for 2 min before holding at 4° C. In 50 μL reaction volume, the PCR mix included 1×PCR buffer (Invitrogen), 1.5 mM MgCl₂, 0.25 mM dNTPs, 25 ng human gDNA (Promega #G1471), 0.5 μM each forward and reverse primers, and 2.5 U of the polymerase. The PCR products were resolved on 1% agarose gel.

Reverse transcription within droplets: Total RNA from brain tissue (ThermoFisher AM7962) 5 μg was mixed with forward and reverse primers targeting the BEGAIN gene in Enzchek RT buffer. The RNA-primer mixture was then subjected to a dolomite microfluidic device to generate water-in-oil emulsion droplets. The device produced approximately 3 million droplets with an average diameter of 20 μm and a total input volume of 100 μL. Half of the generated droplets (50 μL) were transferred to a thermocycler and underwent reverse transcription and PCR amplification using the following protocol: 55° C. for 30 minutes, followed by 35 cycles of 95° C. for 30 seconds, 55° C. for 30 seconds, and 72° C. for 40 seconds. After thermal cycling, 2 μL of the post-PCR droplets were mixed with 2 μL of 2× concentrated Picogreen dye on a microscope slide. Following a 10-minute incubation at room temperature, the sample was examined under a fluorescence microscope. The same microscopy procedure was applied to pre-PCR droplets as a comparison to highlight the reverse transcription and amplification, with the post-PCR droplets expected to exhibit fluorescence while the pre-PCR droplets would not. A negative control was also included, where everything was performed the same but no brain total RNA was added from the beginning.
Reverse transcription and RT-PCR from cells in organic cosolvents: Two-Step RT-PCR on Synthesized Partial KRAS RNA. A 119-nucleotide partial KRAS RNA (sequence: AGCUAAUUCAGAAUCAUUUUGUGGACGAAUAUGAUCCAACAAUAGAGGAUUCCUACAGGAAG CAAGUAGUAAUUGAUGGAGAAACCUGUCUCUUGGAUAUUCUCGACACAGCAGGUCAA) was synthesized by Genscript. Fifty picomoles of the KRAS RNA were mixed with 50 pmol of forward and reverse primers, and 0.5 mM dNTPs in EnzChek RT buffer. Different reverse transcriptases, either purified enzymes or unpurified preparations, were then added to the reaction mixture. These reverse transcriptases included: A—SFM4-6 (40 ng, positive control), B, C, D—5 million bacterial cells expressing wild-type Taq, SFM4-6, and L-5-2-F01-RT1, respectively. Four sets of the above mixtures were assigned different concentrations of 1,4-butanediol (BD) (0%, 5%, 10%, 20%). The RT reaction mixture (final volume 20 μL) was then transferred to a PCR thermocycler and incubated at 80° C. for 10 minutes followed by 55° C. for 60 minutes. After the RT reaction, the samples were centrifuged before being used for the subsequent PCR step. PCR was carried out using the NEB LUNA PCR mix (Cat. 3003S, 20 μL final volume) with 0.5 μL of the completed RT reaction mixture as the template. The PCR products were analyzed on a 2.5% agarose gel.
Data Tables Tables referenced in the specification hereinabove are provided below:

### Thermostable and Solvostable Reverse Transcriptases: Solvophilicity

TABLE 1
The performance of the top engineered polymerases in terms of DNA-dependent DNA polymerase activity, reverse transcriptase
(RT) activity, thermal stability, and amplification efficiency. The top hit clones were selected from combined screening
of epPCR and shuffled libraries. The effect of 1,4-butanediol (BD) on specific activity, half life (Table 5B), PCR efficiencies (Cqs) on
templates of varying GC contents (WT-Taq and c-Jun templates) (Table 9), and reverse transcriptase activity (Table 2) was measured;
several properties were measured in other cosolvents as well, including tetramethylene sulfoxide (TMSO), sulfolane, and 2-pyrrolidone.

| | | Specific DNA | | | | | | | | | | |
|---|---|---|---|---|---|---|---|---|---|---|---|---|
| | | Polymerase | | | | Amplification | | | | | | |
| | | Activity | | Thermal stability | | Efficiency | | Molar RT Activity | | | | |
| | | (mU/ng) | | (t_{1/2}, min) | | in 7% BD | | (mU/μmol ×10⁸, 68° C.) | | | | |
| | | 72° C., | | 97.5° C., | | (Cq, cycles) | | 0% | | | | |
| | | 0% | 72° C., | 0% | 97.5° C., | WT- | | Co- | 10% | 20% | 14% | 20% |
| Clone | Mutations | cosolvent | 5% BD | cosolvent | 5% BD | Taq | c-Jun | solvent | BD | BD | TMSO | TMSO |
| N-7-3-C08 | F4821, Q534R, | 172.7 | 55.8 | 57.4 ± 7.9 | 22.8 ± 1.2 | 8.7 ± 0.6 | 10.3 ± 0.2 | NA | NA | NA | NA | NA |
| | A609V, F749R | | | | | | | | | | | |
| N-7-2-E02 | F73S, A118V, | 167.3 | 50.4 | 46.4 ± 8.0 | 19.6 ± 2.0 | 8.4 ± 0.6 | 10.2 ± 0.2 | NA | NA | NA | NA | NA |
| | F749I | | | | | | | | | | | |
| N-7-3-B07 | A23P, L162P, | 149.4 | 41 | 49.6 ± 2.5 | 14.7 ± 2.5 | 8.4 ± 0.5 | 9.8 ± 0.2 | NA | NA | NA | NA | NA |
| | I228V, L461R, | | | | | | | | | | | |
| | A521V, E734G, | | | | | | | | | | | |
| | F749I, L768M | | | | | | | | | | | |
| N-7-3-G09 | L5Q, A23P, F749I | 158.8 | 58 | ND | ND | ND | ND | NA | NA | NA | NA | NA |
| L-5-2-F01 | A97T, A608V, | 139.8 | 94.7 | 101 ± 14.6 | 111 ± 12.6 | 8.1 ± 0.5 | 10.0 ± 0.2 | NA | NA | NA | NA | NA |
| | K702R, K762R | | | | | | | | | | | |
| L-5-2-F08 | E434D, E507K, | 193.6 | 122 | ND | ND | ND | ND | NA | NA | NA | NA | NA |
| | K762R | | | | | | | | | | | |
| L-5-3-A08 | E434D, A608V, | 193.1 | 95.7 | ND | ND | ND | ND | NA | NA | NA | NA | NA |
| | K762R | | | | | | | | | | | |
| L-5-3-D04 | P10S, E507K, | 99.8 | 74.7 | 68.0 ± 4.1 | 31.3 ± 3.4 | 8.0 ± 0.4 | 9.9 ± 0.1 | NA | NA | NA | NA | NA |
| | Q680R ,K762R | | | | | | | | | | | |
| SPC9 | P10S, A61V, | 39.1 | 42.6 | ND | ND | 8.3 ± 0.5 | 10.2 ± 0.2 | NA | NA | NA | NA | NA |
| | T186I, D244V, | | | | | | | | | | | |
| | K314R, E520G, | | | | | | | | | | | |
| | V586A, S612R, | | | | | | | | | | | |
| | V730I, F749V | | | | | | | | | | | |
| WT Taq | | 119.5 | 30.4 | 3.7 ± 1.5 | 0.8 ± 0.0 | >20 | >20 | NA | NA | NA | NA | NA |
| L-5-2- | L-5-2-F01 + | 176.6 | 72 | ND | ND | ND | ND | 142.0 | 768.1 | 2707.6 | 1852.2 | 2413.1 |
| F01-RT1 | E742K + | | | | | | | | | | | |
| | M747K | | | | | | | | | | | |
| N-7-3- | N-7-3-B07 + | 94.6 | 13.5 | ND | ND | ND | ND | 56.3 | 75.5 | ND | ND | ND |
| B07-RT | E742K + | | | | | | | | | | | |
| | M747K | | | | | | | | | | | |
| L-5-2- | L-5-2-F01 + | 141.9 | 66.4 | ND | ND | ND | ND | 34.3 | 47.8 | 70.1 | 38.1 | 181.4 |
| F01-RT2 | D732N | | | | | | | | | | | |
| ProtoScript | | 0 | 0 | 0 | 0 | NA | NA | 0 | 0 | 0 | 0 | 0 |
| II RT | | | | | | | | | | | | |

ND: not determined; NA: not applicable under the conditions of the experiments.

TABLE 2
RT and RT-qPCR activity of thermostable reverse transcriptases in organic cosolvents. Based on RT-qPCR results with engineered
Taq polymerases (L-5-2-F01-RT1, N-7-3-B07-RT, SFM4-6; FIG. 5I, K), it was observed that reverse transcription in BD/2-
pyrrolidone/Sulfolane/Tetramethylene sulfoxide succeeded for enzymes engineered in BD (L-5-2-F01-RT1, N-7-3-B07-RT).
Hence comparison to NEB Protoscript II employed 7% BD for L-5-2-F01-RT1, N-7-3-B07-RT but 0% BD for NEB
Protoscript II (FIG. 5). Target peak height is expressed as 100%*[-d(RFU)/dT-baseline]/max, where max denotes-d(RFU)/
dT-baseline for the highest peak. ΔTₘ denotes the difference in peak T_{M} between the peak and the max height peak (FIG. 5K).
Target and nonspecific bands refer to primary and secondary peaks in FIG. 5L. RT-qPCR conditions employed in FIG. 5I, K
led to a single major peak with nonspecificity producing peak broadening and a downshift in Tₘ, whereas conditions
employed in FIG. 5J, L led to two peaks with nonspecificity producing a larger secondary peak at lower T_{M}.

| | | | | | | RT-qPCR Target | | | |
|---|---|---|---|---|---|---|---|---|---|
| | Molar RT Activity, mU/μmol × 10⁸ | | | | | Peak Height* | | | |
| | Poly A template | | | | | BEGAIN template | | RT-qPCR Peak Height** | |
| | 55° C. | | 68°C | | BEGAIN | ΔTm | | BEGAIN template | |
| | | 10% BD | | 10% BD | template | 55° C. | | Target, Nonspecific Peaks | |
| | | 10% 2-pyrrolidone | | 14% 2-pyrrolidone | 55° C. | RT with | | 55° C. | 68° C. |
| | 0% co- | 10% sulfolane | 0% co- | 14% sulfolane | 0% | 0% | RT with | L-5-2-F01-RT: RT 7% BD | |
| Clone | solvent | 14% TMSO | solvent | 14% TMSO | cosolvent | cosolvent | 7% BD | Protoscript II: RT 0% BD | |
| L-5-2-F01-RT1 | 405.1 | 942.6 | 141.9 | 768.1 | ND | 72.4% | 100%; | 100%, 16.1% | 83.4%, 10.3% |
| A97T, A608V, | | 751.7 | | 1229.8 | | −1.5° C. | 0° C. | | |
| K702R, K762R, | | 1016.3 | | 576.8 | | | | | |
| E742K, M747K | | 1358.1 | | 1852.2 | | | | | |
| N-7-3-B07-RT | 81.3 | | 56.3 | 75.5 | ND | 56.7%; | 73.2%; | ND | ND |
| A23P, L162P, | | 122.4 | | | | −1.5° C. | 0° C. | | |
| 228V, L461R, | | ND | | ND | | | | | |
| A521V, E734G, | | | | | | | | | |
| F749I, L768M, | | ND | | ND | | | | | |
| E742K, M747K | | ND | | ND | | | | | |
| SFM 4-6 | 446.1 | 616.3 | 309.1 | 414.7 | 128.6 | 37.4%; | 30.3%; | ND | ND |
| I614E, E615G, | | 269.3 | | 358.2 | | −2.5° C. | −7.5° C. | | |
| D655N, L657M, | | 471.8 | | 310.9 | | | | | |
| E681K, E742N, | | 272.5 | | 400.7 | | | | | |
| M747R | | | | | | | | | |
| NEB Protoscript II | 419.9 | ND | ND | ND | 83.9 | ND | ND | 89.7%, 21.0% | 0%, 27.4% |

ND = Not Determined.
*PCR by L-5-2F01 (FIG. 5K);
**PCR by L-5-2-F01 (FIG. 5L)

TABLE 3
Combined screening of epPCR and shuffled libraries. Top hit clones from 1^{st}, 7^{th} CSR epPCR and
5^{th} CSR of shuffled library clones were grown in a single 96-well plate to compare the PCR performance
in two different conditions. Cells were grown as described in Methods. PCR was run in 5% BD -
95° C. for 6 min, followed by 16 cycles of 94° C. for 30 sec, 57.8° C. for 30
sec, 72° C. for 30 sec, or in 7% BD- 98.3° C. for 1 min, 95° C. for 6 min, followed
by 16 cycles of 94° C. for 30 sec, 57.8° C. for 30 sec, 72° C. for 30 sec. In
both cases, final extension was done at 72° C. for 2 min before holding at 4° C. We
used 30 uL PCR product and mixed with 1X SYBR Green I to run melt-curve to determine the DNA
product peak area. The melt-curve area was normalized by the total number of cells.

| CSR | | | Normalized peak | Normalized peak |
|---|---|---|---|---|
| Rounds | Clones | Mutation positions relative to WT-Taq | area (5% BD) | area (7% BD) |
| | WT | None | 100.44 ± 23.59 | 21.99 ± 2.60 |
| Top | N-1-1-G09 | A61V | 197.18 ± 0.80 | 31.85 ± 8.50 |
| clones- | L-1-23-H10 | E520G, V586A, S612R | 188.48 ± 6.24 | 25.81 ± 3.32 |
| Round 1 | N-1-5-E09 | F749V | 327.26 ± 41.38 | 38.40 ± 6.89 |
| | L-1-14-H10 | V586A, S612R | 195.06 ± 33.17 | 35.24 ± 0.62 |
| | L-1-17-A09 | L30P | 189.54 ± 26.73 | 53.11 ± 23.79 |
| Top | N-7-1-E10 | A29T, G200S, D237G, F749I | 271.54 ± 15.71 | 33.03 ± 12.34 |
| clones - | N-7-1-F06 | L16P, F73S, E388D, G396D, Q680R, F749I | 282.03 ± 35.08 | 63.90 ± 3.93 |
| epPCR | N-7-3-C08 | F482I, Q534R, A608V, F749I | 295.32 ± 40.64 | 207.41 ± 19.39 |
| (Gen 1) | N-7-2-E02 | F73S, A118V, F749I | 313.65 ± 31.51 | 195.56 ± 3.13 |
| Round 7 | N-7-4-D06 | F73S, N220D, I503T, S515N, F749V | 275.38 ± 23.64 | 9.29 ± 11.84 |
| | N-7-4-F04 | A29T, S290G, L461R, D551G, L606M, S739G, F749I | 321.12 ± 14.36 | 12.91 ± 15.39 |
| | N-7-3-B07 | A23P, L162P, I228V, L461R, A521V, E734G, F749I, | 349.27 ± 0.00 | 14.94 ± 18.70 |
| | | L768M | | |
| Top | L-5-3-A11 | E434D, E507K, E742K, F749I | 174.01 ± 27.44 | 177.24 ± 54.78 |
| clones - | L-5-1-C02 | P10S, P382T, E434D, E507K | 257.57 ± 1.33 | 245.31 ± 56.70 |
| Shuffling | L-5-3-H08 | R205K, K219E, E434D, V474I, A608V, INS661R, | 256.17 ± 12.40 | 219.67 ± 16.54 |
| (Gen 2) | | E742K, F749I | | |
| Round 5 | L-5-2-F01 | A97T, A608V, K702R, K762R | 281.77 ± 6.32 | 297.06 ± 37.81 |
| | L-5-2-A03 | F8L, P10S, E434D, E507K, K762R, K767R | 290.93 ± 24.94 | 289.57 ± 12.38 |
| | L-5-3-D04 | P10S, E507K, Q680R, K762R | 309.26 ± 27.01 | 297.72 ± 1.54 |
| | L-5-3-E10 | E507K, A608V, Q782H, F749I | 276.52 ± 25.20 | 278.88 ± 11.69 |
| | L-5-3-D10 | E434D, A608V, E742K, F749I | 250.06 ± 34.69 | 233.10 ± 23.69 |
| Synthetic | SPC9 | P10S, A61V, T186I, D244V, K314R, E520G, V586A, | 241.20 ± 2.11 | 75.62 ± 12.05 |
| | | S612R, V730I, F749V | | |

NGS-Based Identification of Taq Polymerase Mutations that May Confer Reverse Transcriptase Activity

TABLE 4
Frequency of top 15 fragments as scored by next-generation sequencing after each CSR round of the
(A) N-epPCR libraries and (B) L-StEP libraries. We conducted next-generation sequencing after each
enrichment CSR round of the epPCR and StEP libraries. Frequency of each unique mutation species within
a region was calculated by dividing the number of reads of specific mutation by the total number of
detected reads of sequences within that region and multiplying by 100 to yield percentage.
(A) N-epPCR libraries

| | N-epPCR | 1st CSR | 2nd CSR | 3rd CSR | 4th CSR | 5th CSR | 6th CSR | 7th CSR |
|---|---|---|---|---|---|---|---|---|
| MUTATIONS | (gen1) | Round | Round | Round | Round | Round | Round | Round |
| [‘F749I’] | 0.0495 | 0.0542 | 1.4743 | 2.9412 | 13.3875 | 18.2843 | 10.8835 | 35.1010 |
| [‘F749V’] | 0.1551 | 0.1478 | 0.7399 | 3.0963 | 8.5384 | 8.4025 | 3.5531 | 8.0670 |
| [‘A29T’] | 0.0190 | 0.0253 | 0.1466 | 4.6592 | 2.9559 | 4.6426 | 1.4713 | 5.8463 |
| [‘F73S’] | 0.0705 | 1.8868 | 4.8544 | 4.0631 | 1.8668 | 2.5533 | 0.8363 | 3.3034 |
| [‘L5Q’, ‘A23P’] | 0.0000 | 0.0000 | 0.0000 | 0.2677 | 0.1393 | 0.4275 | 0.2286 | 1.7939 |
| [‘D320N’] | 1.1154 | 1.3776 | 0.8515 | 1.5723 | 1.1351 | 0.3776 | 0.1880 | 1.2802 |
| [‘E734G’, ‘F749I’] | 0.0000 | 0.0010 | 0.0011 | 0.0011 | 0.2149 | 0.3516 | 0.2810 | 1.2705 |
| [‘L461R’] | 0.0749 | 1.2154 | 1.2214 | 1.2582 | 0.8578 | 0.6414 | 0.3218 | 1.2198 |
| [‘E601D’] | 0.1238 | 0.0585 | 0.0219 | 0.1701 | 1.9568 | 0.1176 | 0.5816 | 1.1822 |
| [‘L287P'] | 0.0600 | 0.0174 | 0.0112 | 0.0336 | 0.4198 | 0.4972 | 0.5785 | 1.0236 |
| [‘A23P’] | 0.0054 | 0.0053 | 0.0036 | 0.0664 | 0.2631 | 0.2905 | 0.1327 | 0.9732 |
| [‘T509A’] | 0.0606 | 0.0123 | 0.0239 | 0.0223 | 0.5072 | 0.7204 | 0.2994 | 0.9730 |
| [‘R205K’, ‘K219E’] | 0.0000 | 0.0000 | 0.0020 | 0.1531 | 0.5036 | 0.5795 | 0.5224 | 0.8984 |
| [‘E465D’] | 0.7665 | 0.9061 | 0.0286 | 0.8826 | 1.1877 | 0.0638 | 0.6720 | 0.8788 |
| [‘E634A’] | 0.0413 | 0.0227 | 0.0360 | 0.0463 | 1.2159 | 0.1904 | 0.2747 | 0.8333 |

(B) L-StEP libraries

| | | 1st CSR | 2nd CSR | 3rd CSR | 4th CSR | 5th CSR |
|---|---|---|---|---|---|---|
| MUTATION | L-StEP | Round | Round | Round | Round | Round |
| [‘E434D’, ‘E507K’] | 0.0000 | 0.0012 | 0.1757 | 9.2082 | 29.4415 | 39.1755 |
| [‘A608V’] | 0.3715 | 1.0732 | 4.0182 | 16.1176 | 19.4364 | 19.0714 |
| [‘E742K’, ‘F749I’] | 0.0000 | 0.0000 | 0.0000 | 0.0320 | 1.6439 | 16.9840 |
| [‘F749L’] | 0.0274 | 0.0339 | 0.2103 | 6.5591 | 14.4111 | 16.9422 |
| [‘P10S’] | 6.4485 | 3.6107 | 4.1062 | 14.1814 | 18.6698 | 15.1147 |
| [‘E434D’] | 0.3730 | 2.3270 | 6.6165 | 28.7690 | 20.4126 | 13.0905 |
| [‘F73S’] | 0.2784 | 0.7479 | 3.5363 | 3.9523 | 4.6754 | 5.4740 |
| [‘R205K’, ‘K219E’, M236T’] | 0.1439 | 0.5668 | 2.5233 | 4.4001 | 3.3920 | 3.7638 |
| [‘K767R’] | 0.0147 | 0.0882 | 0.6446 | 12.5791 | 12.7619 | 3.7321 |
| [‘K762R’] | 0.0105 | 0.0058 | 0.0869 | 0.0362 | 0.6161 | 3.7319 |
| [‘M236T’] | 0.0762 | 0.3506 | 0.7372 | 2.9340 | 2.7491 | 3.2943 |
| [‘P382T’] | 0.0080 | 0.0213 | 0.0335 | 0.0432 | 0.4291 | 2.3432 |
| [‘K219E’, ‘M236T’] | 0.0402 | 0.1655 | 0.6475 | 2.4056 | 1.9611 | 2.3004 |
| [‘R205K’, ‘K219E’] | 0.0572 | 0.1441 | 0.5579 | 1.7251 | 1.7519 | 2.1240 |
| [‘E507K’] | 0.1461 | 0.0121 | 0.8365 | 3.1618 | 3.7016 | 1.8211 |

### Stability-Enhancing Taq Polymerase Mutations

TABLE 5
Stability of the WT and engineered Taq polymerases. (A): Comparison of half-life between His-
tagged WT versus cleaved His-wild type Taq polymerase (ND = not determined). In method 1, the
polymerase alone was heat treated whereas in method 2, we made Taq ternary complex (E-TP-dNTP) then
exposed to temperature. (B) shows the half-lives of the engineered polymerases.
(A)

| Temp | t_{1/2} (min) Method 1 | | t_{1/2} (min) Method 2 | |
|---|---|---|---|---|
| (° C.) | His-Taq | Non-His Taq | His-Taq | Non-His Taq |
| 95° C. | 41.2 ± 1.9 | 39.6 | ND | ND |
| 97.5° C. | 3.7 ± 1.5 | 3.6 | 3.9 | 4.2 |

(B) Half-life of the WT and engineered Taq polymerases. We determined the thermostability (half-life) of
wild type and its variants as described in Materials and Methods at 95° C. and 97.5° C., in the presence and
absence of 5% BD. The half-life (t_{1/2}) of each variant was calculated using method 2; the binary complex
[E-TP] was exposed to heat at specified time followed by activity assay at 72° C.

| | 95° C. | | | | 97.5° C. | | | |
|---|---|---|---|---|---|---|---|---|
| Rank | 0% BD | t_{1/2}, min | 5% BD | t_{1/2}, min | 0% BD | t_{1/2}, min | 5% BD | t_{1/2}, min |
| 1 | L-5-2-F01 | >300 | L-5-2-F01 | ≥120 | L-5-2-F01 | 101.2 ± 14.6 | L-5-2-F01 | 111 ± 12.6 |
| 2 | L-5-3-D04 | >300 | L-5-3-D04 | 113.8 ± 6.3 | L-5-3-D04 | 68.0 ± 4.1 | L-5-3-D04 | 31.3 ± 3.4 |
| 3 | N-7-3-C08 | 288 ± 9.5 | N-7-3-C08 | 68.5 ± 8.8 | N-7-3-C08 | 57.4 ± 7.9 | N-7-3-C08 | 22.8 ± 1.2 |
| 4 | N-7-2-E02 | 287.5 ± 20.8 | N-7-2-E02 | 66.2 ± 3.5 | N-7-2-E02 | 46.4 ± 8.0 | N-7-2-E02 | 19.6 ± 2.0 |
| 5 | N-7-3-B07 | 277.2 ± 7.5 | N-7-3-B07 | 61.9 ± 6.1 | N-7-3-B07 | 49.6 ± 2.5 | N-7-3-B07 | 14.7 ± 2.5 |
| 6 | L-1-17-A09 | >120 | SPC8 | 31.5 ± 3.5 | L-1-17-A09 | 30.4 ± 8.5 | SPC8 | 5.8 ± 0.4 |
| 7 | N-1-01-D05 | 102.1 ± 19.2 | L-1-15-A07 | 27.8 ± 14.0 | SPC8 | 26.8 ± 6.0 | L-1-32-D07 | 4.1 ± 2.0 |
| 8 | N-1-05-E09 | 99.4 ± 12.6 | L-1-17-A09 | 23.8 ± 11.9 | L-1-36-A08 | 19.5 ± 3.1 | N-1-2-G02 | 3.3 ± 0.6 |
| 9 | L-1-15-A07 | 91.3 ± 10.3 | L-1-36-A08 | 21.8 ± 5.0 | L-1-23-H10 | 15.2 ± 2.7 | N-1-05-E09 | 3.0 ± 1.8 |
| 10 | L-1-36-A08 | 89.0 ± 4.6 | N-1-2-G02 | 21.5 ± 6.5 | L-1-15-A07 | 14.4 ± 6.5 | L-1-15-A07 | 2.9 ± 1.4 |
| 11 | L-1-23-H10 | 88.8 ± 16.0 | N-1-1-D05 | 21.2 ± 2.7 | N-1-01-D05 | 13.5 ± 4.9 | L-1-36-A08 | 2.9 ± 0.3 |
| 12 | L-1-22-H02 | 82.3 ± 3.2 | L-1-23-H10 | 19.0 ± 6.1 | N-1-1-G11 | 11.7 ± 4.3 | L-1-14-H10 | 2.9 ± 1.5 |
| 13 | L-1-32-D07 | 77.7 ± 2.5 | L-1-14-H10 | 17.3 ± 1.8 | L-1-32-D07 | 10.7 ± 7.1 | L-1-23-H10 | 2.4 ± 1.6 |
| 14 | N-1-1-G09 | 76.4 ± 1.2 | L-1-22-H02 | 17.0 ± 1.5 | N-1-2-G02 | 10.2 ± 5.2 | N-1-01-D05 | 2.2 ± 0.4 |
| 15 | SPC8 | 71.8 ± 23.0 | L-1-32-D07 | 16.7 ± 4.2 | L-1-22-H02 | 9.9 ± 3.0 | N1-01-G11 | 2.1 ± 0.2 |
| 16 | N-1-1-G11 | 68.3 ± 17.1 | N-1-5-E09 | 15.8 ± 5.3 | L-1-14-H10 | 9.5 ± 3.7 | L-1-22-H02 | 2.0 ± 0.0 |
| 17 | L-1-14-H10 | 65.5 ± 9.8 | N1-1-G11 | 15.4 ± 2.9 | N-1-05-E09 | 8.2 ± 2.8 | L-1-17-A09 | 2.0 ± 1.1 |
| 18 | N-1-2-G02 | 64.3 ± 9.8 | N-1-1-G09 | 13.8 ± 0.4 | SPC4 | 6.8 ± 0.4 | SPC4 | 1.9 ± 0.4 |
| 19 | SPC4 | 44.8 ± 9.5 | SPC4 | 11.0 ± 2.8 | N-1-1-G09 | 4.8 ± 3.0 | N-1-01-G09 | 1.9 ± 0.2 |
| 20 | WT | 41.2 ± 1.9 | WT | 6.4 ± 2.1 | WT | 3.7 ± 1.5 | WT | 0.8 ± 0.0 |

TABLE 6
(A) Melting temperature (T_{M}) profile of the wild type and top clones. We tested the T_{M} of His-tag WT
and top hit clones from N1 series 1^{st}, 7^{th} round and L5 series enrichment in a buffer containing 20 mM
Tris-HCl pH 8.0, 100 mM KCl, 0.1 mM EDTA, 1 mM DTT and 5% glycerol. [BD = 1,4-butanediol]

| | | | Exo-domain | Pol-Domain |
|---|---|---|---|---|
| Taq | | | T_{M} | T_{M} |
| Polymerase | Mutations | 5% BD | (° C.) | (° C.) |
| His-tag WT | NONE | No | 88.86 ± 1.29 | 96.49 ± 0.07 |
| | | Yes | 81.84 ± 0.60 | 94.45 ± 0.03 |
| N-7-1-E10 | A29T, G200S, D237G, F749I | Yes | 86.36 ± 0.17 | 96.66 ± 0.03 |
| N-7-1-F06 | L16P, F73S, E388D, Q680R, F749I | Yes | 78.88 ± 0.64 | 96.6 ± 0.03 |
| N-7-2-E02 | F73S, A118V, F749I | Yes | 90.15 ± 0.27 | 96.61 ± 0.03 |
| N-7-3-B07 | A23P, L162P, I228V, L461R, A521V, | Yes | 81.56 ± 0.09 | 97.36 ± 0.08 |
| | E734G, F749I, L768M | | | |
| N-7-3-C08 | K31R, F482I, Q534R, A608V, F749I | Yes | 86.53 ± 0.21 | 96.81 ± 0.02 |
| N-7-4-D06 | N220D, I503T, S515N, F749V | Yes | 87.00 ± 0.85 | 96.45 ± 0.11 |
| N-7-4-F04 | A29T, F73S, S290G, L461R, D551G, | Yes | 88.46 ± 0.39 | 96.14 ± 0.05 |
| | L606M, S739G, F749I | | | |
| L-5-2-F01 | A97T, A608V, K702R, K762R | Yes | 82.33 ± 0.19 | 100.18 ± 0.11 |
| L-5-3-D04 | P10S, E507K, Q680R, K762R | Yes | 82.72 ± 0.08 | 96.32 ± 0.03 |
| N-1-5-E09 | F749V | Yes | 101.45 ± 0.19 | ND |
| L-5-2-F01-RT1 | A97T, A608V, K702R, K762R, E742K, | No | ND | 92.75 ± 0.06 |
| | M747K | Yes | ND | 91.61 ± 0.00 |
| L-5-2-F01-RT2 | A97T, A608V, K702R, K762R, D732N | No | 86.74 ± 0.08 | 95.91 ± 0.09 |
| | | Yes | 86.89 ± 0.00 | 93.95 ± 0.00 |
| SFM4-6 | I614E, E615G, D655N, L657M, E681K, | No | ND | 87.47 ± 0.06 |
| | E742N, M747R (Stoffel) | Yes | ND | 84.72 ± 0.05 |
| SFM4-3 | I614E, E615G, V518A, N583S, D655N, | No | ND | 87.20 ± 0.04 |
| | E681K, E742Q, M747R (Stoffel) | Yes | ND | 84.93 ± 0.01 |

(B) Polymerase melting temperature dependence on cosolvent concentration for wild type, early round
and selected synthetic clones.

| Polymerase | 0% BD | 1% BD | 2% BD | 3% BD | 5% BD | 7.5% BD | 10% BD |
|---|---|---|---|---|---|---|---|
| WT | 102.96 ± 0.12 | 102.30 ± 0.01 | 101.78 ± 0.18 | 101.13 ± 0.15 | 99.75 ± 0.40 | 99.04 ± 0.24 | 97.75 ± 0.19 |
| His-WT | 103.40 ± 0.2 | 102.48 ± 0.14 | 101.70 ± 0.06 | 101.11 ± 0.17 | 99.96 ± 0.04 | 98.91 ± 0.11 | 97.31 ± 0.15 |
| N-1-1-G9 | 104.05 ± 0.21 | 103.18 ± 0.03 | 102.49 ± 0.09 | 102.17 ± 0.15 | 101 ± 0.25 | 99.92 ± 0.22 | 98.98 ± 0.41 |
| N-1-5-E9 | 105.03 ± 0.33 | 104.04 ± 0.06 | 103.19 ± 0.08 | 102.85 ± 0.23 | 101.45 ± 0.19 | 100.3 ± 0.29 | 99.23 ± 0.19 |
| N-1-1-D5 | 104.48 ± 0.20 | 103.7 ± 0.13 | 103.02 ± 0.08 | 102.39 ± 0.17 | 101.36 ± 0.05 | 100.2 ± 0.07 | 99.53 ± 0.41 |
| L-1-14-H10 | 103.86 ± 0.11 | 103.27 ± 0.30 | 102.11 ± 0.65 | 101.9 ± 0.06 | 100.85 ± 0.06 | 99.66 ± 0.06 | 98.38 ± 0.23 |
| L-1-17-A09 | 104.75 ± 0.08 | 103.78 ± 0.19 | 101.96 ± 0.77 | 102.27 ± 0.18 | 101.22 ± 0.27 | 99.67 ± 0.12 | 98.06 ± 0.07 |
| L-1-23-H10 | 104.89 ± 0.57 | 103.75 ± 0.09 | 102.86 ± 0.06 | 102.14 ± 0.08 | 101.18 ± 0.28 | 99.94 ± 0.28 | 98.4 ± 0.03 |
| SPC1 | 105.96 ± 0.30 | ND | ND | ND | 103.31 ± 0.12 | ND | ND |
| SPC2 | 104.58 ± 0.19 | ND | ND | ND | 101.87 ± 0.13 | ND | ND |
| SPC3 | 105.66 ± 0.11 | ND | ND | ND | 103.12 ± 0.06 | ND | ND |
| SPC4 | 105.01 ± 0.23 | ND | ND | ND | 102.26 ± 0.25 | ND | ND |
| SPC5 | 104.31 ± 0.14 | 103.61 ± 0.12 | 103.04 ± 0.12 | 102.99 ± 0.37 | 101.27 ± 0.31 | 100.05 ± 0.03 | 98.34 ± 0.04 |
| SPC6 | 106.61 ± 1.10 | ND | ND | ND | 104.08 ± 0.35 | ND | ND |
| SPC7 | 104.51 ± 0.39 | ND | ND | ND | 101.76 ± 0.24 | ND | ND |
| SPC8 | 105.53 ± 0.22 | ND | ND | ND | 102.88 ± 0.20 | ND | ND |
| SPC9 | 103.76 ± 51 | 103.46 ± 0.21 | 101.91 ± 0.00 | 100.78 ± 1.33 | 101.52 ± 0.08 | 99.88 ± 0.12 | 98.7 ± 0.11 |

TABLE 7
ΔΔG Calculation. Stability of mutant clones from the final rounds of selection were
computed using MAESTROweb software by introducing mutations into coordinate file PDB: 1TAU.

| | | ΔΔG | ΔΔG (kJ/mol), |
|---|---|---|---|
| | | (kJ/mol), | pol domain |
| Clone ID | Mutations Relative to WT-Taq | all mutations | mutations |
| L-5-2-F01 | A97T, A608V, K702R, K762R | −2.22 | −0.54 |
| N-7-3-C08 | K31R, F482I, Q534R, A608V, F749I | −1.36 | −0.51 |
| L-5-3-D04 | P10S, E507K, Q680R, K762R | −0.64 | −0.11 |
| N-7-4-F04 | A29T, F73S, S290G, L461R, D551G, L606M, S739G, F749I | −0.17 | −0.20 |
| N-7-1-E10 | A29T, G200S, D237G, F749I | 0.06 | −0.40 |
| N-7-1-F06 | L16P, F73S, E388D, Q680R, F749I | 0.07 | −0.14 |
| N-7-4-D06 | N220D, I503T, S515N, F749V | 0.30 | −0.10 |
| N-7-2-E02 | F73S, A118V, F749I | 0.68 | −0.40 |
| N-7-3-B07 | A23P, L162P, I228V, L461R, A521V, E734G, F749I, L768M | 1.43 | 0.20 |

TABLE 8
GC bias of GC-rich templates with engineered polymerases.
GC-rich templates were PCR amplified together with either WT or L-5-2-F01 or N-7-3-B07 mutants under
the same conditions. Gene-specific primers (one set of FWD and REV for one template) were added in the
PCR reactions. A) The PCR mix contained 7.25U of each enzyme in the presence of BD and 5 ng of each
template. The reaction mixture contained 0.75 mM dNTP, 1 mg/mL BSA, 3.5 mM MgCl₂, 0.5 μM of each
primer. B) The PCR mix contained 1.25U of each enzyme in the presence of BD and 5 ng of each template.
The reaction mixture contained 0.25 mM dNTP, 1 mg/mL BSA, 3.5 mM MgCl₂, 0.5 UM of each primer. The
following PCR program were used for both Tables A and B: 98.3° C. for 1 min and 95° C. for 6 min, 25 cycles
of 94° C. for 30 sec, 57.8° C. for 30 sec, 72° C. for 50 sec. PCR products were isolated from 1% agarose gel
electrophoresis and subjected to NGS analysis. Percent frequency of each gene in the PCR pool was
calculated from the total number of read sequences. More details of GC content (%) is presented in FIG. 11.

| | | | | Frequency read sequence in percent (%) | | | |
|---|---|---|---|---|---|---|---|
| | | | Gene | WT-Taq | L-5-2-F01 | | |
| Name of | GC content (%) | | size | polymerase | polymerase | N-7-3-B07 polymerase | |
| Gene | Ave | Max | (~) | 4% BD | 10% BD | 10% BD | 4% BD |

A

| | | | | | | | |
|---|---|---|---|---|---|---|---|
| c-Jun | 64 | 77.5 | 375 | 57.09 | 3.77 | 9.39 | — |
| BEGAIN | 71.3 | 80 | 750 | 15.91 | 31.46 | 16.98 | — |
| DACT3 | 79.2 | 100 | 750 | 0.02 | 13.4 | 24.66 | — |
| PO3F3 | 77.7 | 92.5 | 750 | 0.07 | 2.19 | 13.56 | — |
| BAIP3 | 64.4 | 80 | 750 | 26.91 | 49.17 | 35.42 | — |

B

| | | | | | | | |
|---|---|---|---|---|---|---|---|
| pASK-Taq | 61 | 70 | 531 | 61 | — | — | 53 |
| c-Jun | 64 | 77.5 | 375 | 39 | — | — | 47 |

TABLE 9
Efficiency of top-ranked Taq mutants on templates of varying GC contents, in the presence of
varying concentrations of 1,4-butanediol (BD). We used equal activities of the WT and engineered
polymerases to assess the amplification efficiencies of the enzymes. The Cq values for the WT and the top
clones selected from generation 1 library (one clone from 1^{st} enrichment and three clones from 7th
enrichment round), two clones from generation 2 after the 5^{th} enrichment, and a synthetic clone SPC9
(Table 3). The following PCR programs were used to amplify (A) WT-Taq template: 95° C. for 6 min, 16
cycles of 94° C. for 30 sec, 57.8° C. for 30 sec, 72° C. for 60 sec, using Q1 and Q2 primers. (B) c-Jun template:
95° C. for 6 min, 16 cycles of 94° C. for 30 sec, 57.8° C. for 30 sec, 72° C. for 60 sec, using J1 and J3 primers. (C)
WT-Taq template: 98.3° C. for 1 min, 95° C. for 6 min, 17 cycles of 94° C. for 30 sec, 57.8° C. for 30 sec, 72° C.
for 60 sec, using Q1 and Q2 primers, and (D) c-Jun template: 98.3° C. for 1 min, 95° C. for 6 min, 16 cycles of
94° C. for 30 sec, 57.8° C. for 30 sec, 72° C. for 60 sec, using J1 and J3 primers. Cq values are from triplicate experiment.

| | | | | | | | | |
|---|---|---|---|---|---|---|---|---|
| % BD | WT enzyme | N-7-3-C08 | N-7-2-E02 | N-7-3-B07 | N-1-5-E09 | SPC-9 | L-5-2-F01 | L-5-3-D04 |

(A) Template: WT-Taq

| | | | | | | | | |
|---|---|---|---|---|---|---|---|---|
| 0 | 9.46 ± 0.52 | 9.44 ± 0.57 | 9.39 ± 0.57 | 9.23 ± 0.68 | 9.38 ± 0.53 | 9.15 ± 0.50 | 9.35 ± 0.40 | 9.35 ± 0.39 |
| 2 | 8.36 ± 0.53 | 8.10 ± 0.49 | 7.93 ± 0.51 | 8.08 ± 0.52 | 8.19 ± 0.66 | 7.74 ± 0.49 | 7.62 ± 0.47 | 7.23 ± 0.45 |
| 4 | 8.48 ± 0.47 | 8.11 ± 0.37 | 7.84 ± 0.47 | 7.83 ± 0.43 | 8.03 ± 0.43 | 7.81 ± 0.46 | 7.56 ± 0.52 | 7.24 ± 0.39 |
| 5 | 8.84 ± 0.73 | 8.10 ± 0.48 | 8.1 ± 0.48 | 8.07 ± 0.56 | 8.30 ± 0.50 | 7.92 ± 0.55 | 7.63 ± 0.44 | 7.52 ± 0.51 |
| 6 | 12.52 ± 1.59 | 8.34 ± 0.45 | 8.19 ± 0.52 | 8.13 ± 0.45 | 8.53 ± 0.40 | 8.08 ± 0.38 | 7.92 ± 0.41 | 7.64 ± 0.38 |
| 7 | NA | 8.66 ± 0.55 | 8.43 ± 0.61 | 8.39 ± 0.48 | 9.14 ± 0.57 | 8.30 ± 0.47 | 8.08 ± 0.51 | 8.00 ± 0.38 |
| 8 | NA | 8.91 ± 0.50 | 8.97 ± 0.43 | 8.70 ± 0.39 | 9.96 ± 0.92 | 8.73 ± 0.41 | 8.26 ± 0.41 | 8.32 ± 0.29 |
| 10 | NA | 10.12 ± 1.38 | 10.9 ± 0.10 | 10.03 ± 0.78 | NA | 9.53 ± 0.73 | 9.45 ± 0.16 | 8.72 ± 0.44 |

(B) Template: c-Jun

| | | | | | | | | |
|---|---|---|---|---|---|---|---|---|
| 0 | NA | 16.89 ± 0.04 | 15.68 ± 0.65 | 15.28 ± 0.56 | NA | 16.80 ± 0.05 | NA | NA |
| 2 | 13.36 ± 0.90 | 12.85 ± 1.34 | 11.99 ± 0.77 | 11.92 ± 0.55 | 12.51 ± 0.70 | 12.66 ± 1.23 | 13 ± 1.36 | 12.62 ± 1.30 |
| 4 | 10.21 ± 0.47 | 9.82 ± 0.41 | 9.28 ± 0.11 | 9.19 ± 0.05 | 9.78 ± 0.14 | 9.75 ± 0.47 | 9.76 ± 0.89 | 9.31 ± 0.52 |
| 5 | 10.53 ± 0.27 | 9.83 ± 0.22 | 9.49 ± 0.05 | 9.30 ± 0.09 | 10.02 ± 0.06 | 9.58 ± 0.22 | 9.51 ± 0.40 | 9.37 ± 0.40 |
| 6 | 13.99 ± 1.37 | 10.09 ± 0.22 | 9.60 ± 0.07 | 9.46 ± 0.09 | 10.28 ± 0.03 | 9.79 ± 0.26 | 9.84 ± 0.37 | 9.52 ± 0.44 |
| 7 | NA | 10.50 ± 0.21 | 10.00 ± 0.11 | 9.96 ± 0.18 | 11.36 ± 0.32 | 10.12 ± 0.11 | 9.99 ± 0.33 | 9.82 ± 0.39 |
| 8 | NA | 10.94 ± 0.12 | 10.90 ± 0.35 | 10.43 ± 0.13 | 14.56 ± 1.42 | 10.85 ± 0.19 | 10.21 ± 0.34 | 10.52 ± 0.53 |
| 10 | NA | NA | 14.21 | 16.47 | NA | 16.29 | 11.73 ± 0.83 | 12.73 ± 0.60 |

(C) Template: WT-Taq

| | | | | | | | | |
|---|---|---|---|---|---|---|---|---|
| 0 | 9.46 ± 0.52 | 9.44 ± 0.57 | 9.39 ± 0.57 | 9.23 ± 0.68 | 9.38 ± 0.53 | 9.15 ± 0.50 | 9.35 ± 0.40 | 9.35 ± 0.39 |
| 2 | 8.36 ± 0.53 | 8.10 ± 0.49 | 7.93 ± 0.51 | 8.08 ± 0.52 | 8.19 ± 0.66 | 7.74 ± 0.49 | 7.62 ± 0.47 | 7.23 ± 0.45 |
| 4 | 8.48 ± 0.47 | 8.11 ± 0.37 | 7.84 ± 0.47 | 7.83 ± 0.43 | 8.03 ± 0.43 | 7.81 ± 0.46 | 7.56 ± 0.52 | 7.24 ± 0.39 |
| 5 | 8.84 ± 0.73 | 8.10 ± 0.48 | 8.1 ± 0.48 | 8.07 ± 0.56 | 8.30 ± 0.50 | 7.92 ± 0.55 | 7.63 ± 0.44 | 7.52 ± 0.51 |
| 6 | 12.52 ± 1.59 | 8.34 ± 0.45 | 8.19 ± 0.52 | 8.13 ± 0.45 | 8.53 ± 0.40 | 8.08 ± 0.38 | 7.92 ± 0.41 | 7.64 ± 0.38 |
| 7 | NA | 8.66 ± 0.55 | 8.43 ± 0.61 | 8.39 ± 0.48 | 9.14 ± 0.57 | 8.30 ± 0.47 | 8.08 ± 0.51 | 8.00 ± 0.38 |
| 8 | NA | 8.91 ± 0.50 | 8.97 ± 0.43 | 8.70 ± 0.39 | 9.96 ± 0.92 | 8.73 ± 0.41 | 8.26 ± 0.41 | 8.32 ± 0.29 |
| 10 | NA | 10.12 ± 1.38 | 10.9 ± 0.10 | 10.03 ± 0.78 | NA | 9.53 ± 0.73 | 9.45 ± 0.16 | 8.72 ± 0.44 |

(D) Template: c-Jun

| | | | | | | | | |
|---|---|---|---|---|---|---|---|---|
| 0 | 15.35 ± 0.95 | 15.71 ± 0.33 | 13.89 ± 0.53 | 13.55 ± 0.20 | 15.26 ± 1.64 | 15.99 ± 0.34 | 16.91 | 16.53 |
| 2 | 12.76 ± 0.48 | 13.21 ± 0.23 | 12.22 ± 0.07 | 12.28 ± 0.21 | 12.14 ± 0.06 | 13.28 ± 0.17 | 14.01 ± 0.15 | 13.7 ± 0.06 |
| 4 | 12.23 ± 0.59 | 10.17 ± 0.13 | 9.44 ± 0.08 | 9.34 ± 0.02 | 10.06 ± 0.17 | 10.28 ± 0.17 | 10.02 ± 0.33 | 10.05 ± 0.29 |
| 5 | NA | 9.96 ± 0.15 | 9.45 ± 0.13 | 9.21 ± 0.14 | 10.15 ± 0.16 | 9.86 ± 0.21 | 9.67 ± 0.16 | 9.68 ± 0.18 |
| 6 | NA | 10.23 ± 0.14 | 9.81 ± 0.14 | 9.36 ± 0.19 | 11.70 ± 0.83 | 9.95 ± 0.18 | 10.03 ± 0.16 | 9.73 ± 0.14 |
| 7 | NA | 10.32 ± 0.21 | 10.23 ± 0.18 | 9.84 ± 0.19 | NA | 10.15 ± 0.17 | 10.02 ± 0.15 | 9.87 ± 0.14 |
| 8 | NA | 10.95 ± 0.13 | 11.95 ± 1.85 | 10.51 ± 0.35 | NA | 11.01 ± 0.37 | 10.38 ± 0.36 | 10.86 ± 0.55 |
| 10 | NA | NA | NA | NA | NA | NA | 11.32 ± 0.41 | NA |

NA = we did not observe Cq value up to 16th PCR cycle.

TABLE 10
Efficiency of WT-Taq and the mutant, L-5-2-F01 in presence of
varying concentration of cosolvent 2-pyrrolidone or sulfolane:
We used equal activities of the WT and a mutant, L-5-2-F01 to
assess the amplification efficiencies of the enzymes. Cq values
were averaged ±SD from triplicate experiment. NA = we
did not observe Cq value above background. TOP: 2-pyrrolidone;
c-Jun template. BOTTOM: sulfolane; c-Jun template. In both the
cases, PCR was conducted either at 95° C. for 6 min or
98.3° C. for 1 min + 95° C. for 6 min followed
by 16 cycles of 94° C. for 30 sec, 57.8° C. for 30
sec, 72° C. for 60 sec, using J1 and J3 primers.

| | | | 98.3° C. for 1 min, | |
|---|---|---|---|---|
| 2-pyrrolidone | 95° C. for 6 min | | 95° C. for 6 min | |
| (%) | WT | L-5-2-F01 | WT | L-5--2-F01 |
| 0 | 15.42 ± 0.17 | 16.25 + 0.11 | 13.26 | 14.73 + 0.06 |
| 0.5 | 14.45 + 0.19 | 14.92 + 0.22 | NA | 13.64 + 0.47 |
| 1 | NA | 12.21 + 0.13 | NA | 11.98 + 0.15 |
| 1.5 | NA | 12.01 + 0.16 | NA | 11.57 + 0.17 |
| 2 | NA | 10.59 + 0.06 | NA | 10.58 + 0.59 |
| 2.5 | NA | 10.55 + 0.14 | NA | 10.34 + 0.22 |
| 3 | NA | 10.65 + 0.15 | NA | 10.07 + 0.15 |
| 3.5 | NA | 10.66 + 0.07 | NA | 10.26 + 0.08 |
| 4 | NA | 10.60 + 0.06 | NA | 10.19 + 0.15 |
| 4.5 | NA | 10.45 + 0.23 | NA | 10.22 + 0.20 |
| 5 | NA | 10.73 + 0.07 | NA | 10.45 + 0.10 |

| | | | 98.3° C. for 1 min, | |
|---|---|---|---|---|
| sulfolane | 95° C. for 6 min | | 95° C. for 6 min | |
| (%) | WT | L-5-2-F01 | WT | L-5-2-F01 |
| 0 | NA | NA | 15.35 + 0.79 | 16.36 + 0.11 |
| 1 | 12.93 + 0.30 | 13.81 + 0.25 | NA | 13.68 + 0.59 |
| 2 | NA | 11.64 + 0.18 | NA | 11.84 + 0.30 |
| 3 | NA | 11.44 + 0.10 | NA | 11.48 + 0.23 |
| 4 | NA | 11.69 + 0.17 | NA | 11.76 + 0.16 |
| 5 | NA | 12.07 + 0.13 | NA | 12.12 + 0.20 |
| 6 | NA | 12.6 + 0.39 | NA | 12.81 + 0.55 |
| 7 | NA | 13.77 + 0.28 | NA | 16.55 |
| 8 | NA | 15.9 | NA | NA |
| 9 | NA | NA | NA | NA |
| 10 | NA | NA | NA | NA |

TABLE 11
Fidelity of the WT polymerase and mutant derivatives. We determined
the fidelity of the top clones apparent error was calculated
using the formula described in Materials and Methods. We used
WT and variant Taq polymerase to amplify the entire LacZ gene,
followed by apparent error rate assessment. T and W represents
the number of total and white colonies respectively. Apparent
error (Error app.) is given in changes per base pair incorporated
e.g., in case of WT, one nucleotide change is expected per
19173 nucleotides incorporated. Fidelity of the WT was assessed
twice independently.

| Clones | Total | Blue | White | (W/T)*100 | Error app. |
|---|---|---|---|---|---|
| WT | 2414 | 2016 | 398 | 16.5 | 1/19173 |
| WT | 4111 | 3654 | 457 | 12.5 | 1/14062 |
| N-1-5-E09 | 5927 | 5535 | 392 | 7.1 | 1/17802 |
| SPC9 | 4578 | 3797 | 781 | 10.6 | 1/11006 |
| N-7-3-C08 | 5058 | 4671 | 387 | 8.3 | 1/18004 |
| N-7-2-E02 | 4259 | 3937 | 322 | 8.2 | 1/19697 |
| N-7-3-B07 | 5022 | 4599 | 423 | 9.2 | 1/16564 |
| L-5-2-F01 | 5027 | 4659 | 368 | 7.9 | 1/15522 |
| L-5-3-D04 | 5972 | 5550 | 422 | 7.6 | 1/16366 |

TABLE 12
Effect of 1,4-butanediol (BD) on specific activities of the WT and engineered Taq polymerases.
Proteins were purified and quantified as described. Equal amount of proteins were used to
assess the primer extension activity of the enzymes in absence and in presence of 5% BD.

| Libraries and | | | Specific | | |
|---|---|---|---|---|---|
| enrichment rounds/ | | | Activity (mU/ng) | | |
| CLONE ID | | Unique Mutations | 0% BD | 5% BD | % Loss |
| Gen 1, | WT | NONE | 119.5 | 30.39 | 74.57 |
| Round 1 | N1-1-G11 | K206Q | 75.28 | 54.09 | 28.14 |
| | N-1-1-G09 | A61V | 55.91 | 38.90 | 30.43 |
| | N-1-1-G05 | E832K | 115.85 | 82.66 | 28.65 |
| | N-1-1-G07 | L365Q | 104.52 | 65.03 | 37.78 |
| | N-1-2-G04 | T186I | 119.87 | 95.42 | 20.39 |
| | N-1-2-C11 | P10S | 58.47 | 46.19 | 21 |
| | N-1-1-B01 | A54V | 86.24 | 69.29 | 19.66 |
| | N-1-5-E09 | F749V | 67.97 | 54.70 | 19.52 |
| | N-1-2-G02 | D244V, K314R, V586A, S612R | 62.13 | 44.98 | 27.6 |
| | N-1-5-H7- | F667Y | 69.07 | 45.59 | 34 |
| | M1 | | | | |
| | N-1-5-H7- | F749Y | 66.51 | 46.19 | 30.55 |
| | M2 | | | | |
| Gen 2, | L-1-36-A08 | E434D | 42.39 | 33.43 | 21.14 |
| Round 1 | L-1-15-A07 | P10S, V730I | 65.78 | 40.11 | 39.02 |
| | L-1-22-H02 | A54V | 82.60 | 62.22 | 23.46 |
| Gen 2, | L-5-2-F01 | A97T, A608V, K702R, K762R | 139.80 | 94.70 | 32.26 |
| Round 5 | L-5-3-D04 | P10S, E507K, Q680R, K762R | 99.80 | 74.70 | 25.15 |
| | L-5-2-F08 | E434D, E507K, K762R | 193.57 | 121.95 | 37.00 |
| | L-5-3-A08 | P10S, A608V, K762R | 193.05 | 95.69 | 50.43 |
| Gen 1, | N-7-1-E10 | A29T, G200S, D237G, F749I | 210.19 | 90.44 | 56.97 |
| Round 7 | N-7-1-F06 | L16P, F73S, E388D, G396D, Q680R, F749I | 121.45 | 38.79 | 68.04 |
| | N-7-3-C08 | F482I, Q534R, A608V, F749I | 172.66 | 55.77 | 67.7 |
| | N-7-2-E02 | F73S, A118V, F749I | 167.29 | 50.41 | 69.87 |
| | N-7-4-D06 | F73S, N220D, I503T, S515N, F749V | 99.65 | 57.92 | 41.88 |
| | N-7-4-F04 | A29T, S290G, L461R, D551G, L606M, S739G, | 84.23 | 37.55 | 55.42 |
| | | F749I | | | |
| | N-7-3-B07 | A23P, L162P, I228V, L461R, A521V, E734G, | 149.42 | 41.03 | 72.54 |
| | | F749I, L768M | | | |
| | N-7-3-G09 | L5Q, A23P, F749I | | | |
| Synthetic | SPC1 | P10S, A61V, T186I, V586A, S612R, 2494ΔG | 64.68 | 32.82 | 49.26 |
| clones | SPC2 | P10S, A61V, T186I, V586A, S612R | 61.03 | 41.33 | 32.28 |
| | SPC4 | P10S, A61V, D244V, S612R, E832K | 59.93 | 43.76 | 26.98 |
| | SPC5 | A54V, A61V, T186I, K314R, E520G, S612R | 50.80 | 31.61 | 37.78 |
| | SPC6 | G12T, A54V, T186I, D244V, F667Y, F749V | 61.39 | 51.66 | 15.85 |
| | SPC7 | P10S, L30P, A61V, L365P, V586A, S612R, | 91.36 | 66.25 | 27.49 |
| | | E832K | | | |
| | SPC8 | L30P, A54V, E434D, K206Q, S612R, V730I, | 43.85 | 40.72 | 7.14 |
| | | F749V | | | |
| | SPC9 | P10S, A61V, T186I, D244V, K314R, E520G, | 39.10 | 42.55 | −8.81 |
| | | V586A, S612R, V730I, F749V | | | |

TABLE 13
Tₘ and GC contents of GC-rich targets employed in the
present work. Templates 12-14, which have lower GC
contents than most of the other templates, were studied
in reference4 without engineered polymerases.

| | | Ave GC | Predicted | |
|---|---|---|---|---|
| No. | Human Gene | content (%) | Tm (° C.) | Gene size (bp) |
| 1 | B3GT6 | 72.8 | 93.8 | 742 |
| 2 | BEGAIN | 71.3 | 93.3 | 776 |
| 3 | CD5R2 | 64.2 | 90.4 | 793 |
| 4 | CDN1C | 77.4 | 95.7 | 720 |
| 5 | CECR6 | 69.6 | 92.5 | 774 |
| 6 | DACT3 | 79.2 | 96 | 722 |
| 7 | F8I2 | 77.9 | 96 | 735 |
| 8 | JAG2 | 57.6 | 87.3 | 774 |
| 9 | PO3F3 | 77.7 | 96 | 790 |
| 10 | BAIP3 | 64.4 | 90.5 | 788 |
| 11 | KLF14 | 71.6 | 93.5 | 777 |
| 12 | FOLH1 (PSM) | 52.4 | 85.1 | 511 |
| 13 | c-Jun | 64.4 | 90.6 | 996 |
| 14 | bovine GTP | 58.4 | 87.8 | 661 |
| 15 | WT-Taq* | 61 | 88.1 | 531 |

*Taq polymerase from Thermus aquaticus.

TABLE 14
Oligonucleotides used in this study.

| Name | Sequence (5′→3′) | Sequence Listing |
|---|---|---|
| Taq-CSR-P1 | CAGGAAACAGCTATGACAAAAATCTAGATAA | SEQ ID NO: 86 |
| | CGAGGGCAA | |
| Taq-CSR-P2 | GTAAAACGACGGCCAGTAGCTTAGTTAGATAT | SEQ ID NO: 87 |
| | CAGAGACCATGGT | |
| Taq-ReAmp-P3 | CAGGAAACAGCTATGAC | SEQ ID NO: 88 |
| Taq-ReAmp-P4 | GTAAAACGACGGCCAGT | SEQ ID NO: 89 |
| CSR-Select-F | GAATAGTTCGACAAAAATCTAGATAACGAGG | SEQ ID NO: 90 |
| | GCAAAAAATG | |
| AU-Gen-Taq-R | CCTGCAGGTCGACTTATTCTTTCGCGCTCAGC | SEQ ID NO: 91 |
| | CAGTC | |
| Primer J1 | ATGACTGCAAAGATGGAAACG | SEQ ID NO: 92 |
| Primer J2 | TCAAAATGTTTGCAACTGCTGCG | SEQ ID NO: 93 |
| Primer J3 | TGTTCTGGCTGTGCAGTT | SEQ ID NO: 94 |
| His-F | CACCACCACCGTGGTATGCTGCCGCTG | SEQ ID NO: 95 |
| His-R | ATGATGATGCATTTTTTGCCCTCGTTATCTAGA | SEQ ID NO: 96 |
| | TTTTTGTC | |
| 6His-TEV-R | TCGTGGTGGTGATGATGATGCATTTTTTGCC | SEQ ID NO: 97 |
| | CTCGTTATCTAGATTTTTGTC | |
| 6His-TEV-Ser-F | GAACCTGTACTTCCAGTCCCGTGGTATGCTG | SEQ ID NO: 98 |
| | CCGCTG | |
| Taq-Q1 | GGTCACCCGTTCAACCTGAACAG | SEQ ID NO: 99 |
| Taq-Q2 | GTCAACCGCCTTCACGCGGAAC | SEQ ID NO: 100 |
| 40M13LFF | FAM- | SEQ ID NO: 101 |
| | GTTTTCCCAGTCACGACGTTGTAAAACGACG | |
| | GCC | |
| NGSR1_FWD | AAA TCT AGA TAA CGA GGG CAA AAA | SEQ ID NO: 102 |
| NGSR1_REV | GTC TGC GGT CAG AAT ACG | SEQ ID NO: 103 |
| NGSR2_FWD | GAG AAA GAA GGT TAC GAG GTT | SEQ ID NO: 104 |
| NGSR2_REV | ACC GAA CTC CAG ACG TTC | SEQ ID NO: 105 |
| NGSR3_FWD | CTG CGT GCG TTC CTG | SEQ ID NO: 106 |
| NGSR3_REV | ACC CCA CAG GTT CGC | SEQ ID NO: 107 |
| NGSR4 FWD | CTG AGC GAA CGT CTG TTC | SEQ ID NO: 108 |
| NGSR4_REV | GGT ACG CGG GTG AAT CAG | SEQ ID NO: 109 |
| NGSR5_FWD | GAC CCG CTG CCG GAC | SEQ ID NO: 110 |
| NGSR5_REV | GTA ACG TTC GAT GAA CGC TTG | SEQ ID NO: 111 |
| NGSR6_FWD | GCG ATT CCG TAC GAG GAA | SEQ ID NO: 112 |
| NGSR6_REV | CCC CTG CAG GTC GAC | SEQ ID NO: 113 |
| SATP* | tagcgaaggatgtgaacctaatcccTGCTCCCGCGGC | SEQ ID NO: 114 |
| | CGatctgcCGGCCGCGGGAGCA | |
| B3GT6_FWD | GACGCCTACGAAAACCTCAC | SEQ ID NO: 115 |
| B3GT6_REV | CCGAGAAGAAGCCCCAGTAG | SEQ ID NO: 116 |
| B3GT6_1_FWD | GCGACGCCTACGAAAACCTC | SEQ ID NO: 117 |
| B3GT6_1_REV | GACGCAGCGACCACAAGC | SEQ ID NO: 118 |
| EGFR_FWD | TCTGGCCACCATGCGAAGC | SEQ ID NO: 119 |
| EGFR_REV | ACCAGTTGAGCAGGTACTGGG | SEQ ID NO: 120 |
| CDN1C_FWD | CCGCAGCACATCCACGAT | SEQ ID NO: 121 |
| CDN1C_REV | CGCAGCGGCATGTCCTGCT | SEQ ID NO: 122 |
| CDN1C_1_REV | ATCCCCGAGTGCAGCTGG | SEQ ID NO: 123 |
| KRAS_FWD | GTATTAAAAGGTACTGGTGGA | SEQ ID NO: 124 |
| KRAS_REV | CTATTGTTGGATCATATTCGTCC | SEQ ID NO: 125 |
| BEGAIN_FWD | CGTCCCCACCTGCGTCA | SEQ ID NO: 126 |
| BEGAIN_REV | CAGCGCTCACGGGGTAGG | SEQ ID NO: 127 |
| BEGAIN_RT- | TGCGGGCCAAGCCGGGGA | SEQ ID NO: 128 |
| FWD | | |
| BEGAIN_RT- | GGTAGGAGTAGGCGCCGATGTCCT | SEQ ID NO: 129 |
| REV | | |
| CD5R2_FWD | TCGTGGAGCCCGACAAG | SEQ ID NO: 130 |
| CD5R2_REV | GGCAGATGGGGACACAGGT | SEQ ID NO: 131 |
| CECR6_FWD | GCTTATCTACTCCATCGCCTTCAC | SEQ ID NO: 132 |
| CECR6_REV | GCCACAGCCAGGGTGTTGA | SEQ ID NO: 133 |
| F812_FWD | GCTGGTATCGAACAAGCTGAAG | SEQ ID NO: 134 |
| F812_REV | GACACCTCGCAGCGGACC | SEQ ID NO: 135 |
| JAG2_FWD | CCCCAAAGTGGACAACCG | SEQ ID NO: 136 |
| JAG2_REV | GCTGGAGCGGGCGTTCT | SEQ ID NO: 137 |
| KLF14_FWD | GCAGGCTCGGAGGTGGG | SEQ ID NO: 138 |
| KLF14_REV | GTCGATGCGGGGAGTTCG | SEQ ID NO: 139 |
| BAIP3_FWD | AGTGCATGGAGGCGGACC | SEQ ID NO: 140 |
| BAIP3_REV | GCCAAGAAGCCCCTTGTGAG | SEQ ID NO: 141 |
| PO3F3_FWD | CGTCCTCTGTCAAGATGGTCC | SEQ ID NO: 142 |
| PO3F3_REV | TTGAACTGCTTGGCGAACTG | SEQ ID NO: 143 |
| DACT3_FWD | GTCGTCGTGGGAGTCGGA | SEQ ID NO: 144 |
| DACT3_REV | CGCTGGAGGCAGAGCTGAA | SEQ ID NO: 145 |

*Underlined lowercase forms the overhang for the primer extension.

TABLE 15
Reverse Transcriptases Developed Herein
Table 15 provided non-limiting examples of reverse
transcriptases having properties described herein.

| Sequence Listing | Amino acid alterations/mutations |
|---|---|
| SEQ ID NO: 1 | WT-Taq DNA polymerase |
| SEQ ID NO: 2 | SEQ ID NO: 1 incorporating the amino acid alterations amino |
| | acid alterations A97T, A608V, K702R, K762R, E742K, M747K |
| SEQ ID NO: 3 | SEQ ID NO: 1 incorporating the amino acid alterations amino |
| | acid alterations A97T, A608V, K702R, K762R, and D732N |
| SEQ ID NO: 4 | SEQ ID NO: 1 incorporating the amino acid alterations amino |
| | acid alterations A23P, L162P, 228V, L461R, A521V, E734G, |
| | F749I, L768M, E742K, and M747K |
| SEQ ID NO: 5 | SEQ ID NO: 1 incorporating the amino acid alterations amino |
| | acid alterations L30P, A54V, E434D, K206Q, S612R, V730I, |
| | F749V, and D732N |
| SEQ ID NO: 6 | SEQ ID NO: 1 incorporating the amino acid alterations amino |
| | acid alterations L30P, A54V, E434D, K206Q, S612R, V730I, |
| | F749V, E742K, and M747K. |
| SEQ ID NO: 7 | SEQ ID NO: 1 incorporating the amino acid alterations amino |
| | acid alterations L30P, A54V, E434D, K206Q, S612R, V730I, |
| | F749V, E742R, and M747R. |
| SEQ ID NO: 8 | SEQ ID NO: 1 incorporating the amino acid alterations amino |
| | acid alterations L30P, A54V, E434D, K206Q, S612R, V730I, |
| | F749V, E742N, and M747N. |
| SEQ ID NO: 9 | SEQ ID NO: 1 incorporating the amino acid alterations amino |
| | acid alterations L30P, A54V, E434D, K206Q, S612R, V730I, |
| | F749V, E742K, and M747R. |
| SEQ ID NO: 10 | SEQ ID NO: 1 incorporating the amino acid alterations amino |
| | acid alterations L30P, A54V, E434D, K206Q, S612R, V730I, |
| | F749V, E742K, and M747N. |
| SEQ ID NO: 11 | SEQ ID NO: 1 incorporating the amino acid alterations amino |
| | acid alterations L30P, A54V, E434D, K206Q, S612R, V730I, |
| | F749V, E742R, and M747K. |
| SEQ ID NO: 12 | SEQ ID NO: 1 incorporating the amino acid alterations amino |
| | acid alterations L30P, A54V, E434D, K206Q, S612R, V730I, |
| | F749V, E742R, and M747N. |
| SEQ ID NO: 13 | SEQ ID NO: 1 incorporating the amino acid alterations amino |
| | acid alterations L30P, A54V, E434D, K206Q, S612R, V730I, |
| | F749V, E742N, and M747K. |
| SEQ ID NO: 14 | SEQ ID NO: 1 incorporating the amino acid alterations amino |
| | acid alterations L30P, A54V, E434D, K206Q, S612R, V730I, |
| | F749V, E742N, and E747R. |
| SEQ ID NO: 15 | SEQ ID NO: 1 incorporating the amino acid alterations P10S, |
| | A61V, T186I, D244V, K314R, E520G, V586A, S612R, V730I, |
| | F749V, and D732N. |
| SEQ ID NO: 16 | SEQ ID NO: 1 incorporating the amino acid alterations P10S, |
| | A61V, T186I, D244V, K314R, E520G, V586A, S612R, V730I, |
| | F749V, E742K, and M747K. |
| SEQ ID NO: 17 | SEQ ID NO: 1 incorporating the amino acid alterations P10S, |
| | A61V, T186I, D244V, K314R, E520G, V586A, S612R, V730I, |
| | F749V, E742R, and M747R. |
| SEQ ID NO: 18 | SEQ ID NO: 1 incorporating the amino acid alterations P10S, |
| | A61V, T186I, D244V, K314R, E520G, V586A, S612R, V730I, |
| | F749V, E742N, and M747N. |
| SEQ ID NO: 19 | SEQ ID NO: 1 incorporating the amino acid alterations P10S, |
| | A61V, T186I, D244V, K314R, E520G, V586A, S612R, V730I, |
| | F749V, E742K, and M747R. |
| SEQ ID NO: 20 | SEQ ID NO: 1 incorporating the amino acid alterations P10S, |
| | A61V, T186I, D244V, K314R, E520G, V586A, S612R, V730I, |
| | F749V, E742K, and M747N. |
| SEQ ID NO: 21 | SEQ ID NO: 1 incorporating the amino acid alterations P10S, |
| | A61V, T186I, D244V, K314R, E520G, V586A, S612R, V730I, |
| | F749V, E742R, and M747K. |
| SEQ ID NO: 22 | SEQ ID NO: 1 incorporating the amino acid alterations P10S, |
| | A61V, T186I, D244V, K314R, E520G, V586A, S612R, V730I, |
| | F749V, E742R, and M747N. |
| SEQ ID NO: 23 | SEQ ID NO: 1 incorporating the amino acid alterations P10S, |
| | A61V, T186I, D244V, K314R, E520G, V586A, S612R, V730I, |
| | F749V, E742N, and M747K. |
| SEQ ID NO: 24 | SEQ ID NO: 1 incorporating the amino acid alterations P10S, |
| | A61V, T186I, D244V, K314R, E520G, V586A, S612R, V730I, |
| | F749V, M742M, and E747R. |
| SEQ ID NO: 25 | SEQ ID NO: 1 incorporating the amino acid alterations G12T, |
| | A54V, T186I, D244V, F667Y, F749V, and D732N. |
| SEQ ID NO: 26 | SEQ ID NO: 1 incorporating the amino acid alterations G12T, |
| | A54V, T186I, D244V, F667Y, F749V, E742K, and M747K. |
| SEQ ID NO: 27 | SEQ ID NO: 1 incorporating the amino acid alterations G12T, |
| | A54V, T186I, D244V, F667Y, F749V, E742R, and M747R. |
| SEQ ID NO: 28 | SEQ ID NO: 1 incorporating the amino acid alterations G12T, |
| | A54V, T186I, D244V, F667Y, F749V, E742N, and M747N. |
| SEQ ID NO: 29 | SEQ ID NO: 1 incorporating the amino acid alterations G12T, |
| | A54V, T186I, D244V, F667Y, F749V, E742K, and M747R. |
| SEQ ID NO: 30 | SEQ ID NO: 1 incorporating the amino acid alterations G12T, |
| | A54V, T186I, D244V, F667Y, F749V, E742K, and M747N. |
| SEQ ID NO: 31 | SEQ ID NO: 1 incorporating the amino acid alterations G12T, |
| | A54V, T186I, D244V, F667Y, F749V, E712R, and M747K. |
| SEQ ID NO: 32 | SEQ ID NO: 1 incorporating the amino acid alterations G12T, |
| | A54V, T186I, D244V, F667Y, F749V, E742R, and M747N. |
| SEQ ID NO: 33 | SEQ ID NO: 1 incorporating the amino acid alterations G12T, |
| | A54V, T186I, D244V, F667Y, F749V, E742N, and M747K. |
| SEQ ID NO: 34 | SEQ ID NO: 1 incorporating the amino acid alterations G12T, |
| | A54V, T186I, D244V, F667Y, F749V, E742N, and E747R. |
| SEQ ID NO: 35 | SEQ ID NO: 1 incorporating the amino acid alterations P10S, |
| | A61V, F73S, T186I, R205K, K219E, M236T, A608V, S612R, |
| | 2494ΔG, and D732N. |
| SEQ ID NO: 36 | SEQ ID NO: 1 incorporating the amino acid alterations P10S, |
| | A61V, F73S, T186I, R205K, K219E, M236T, A608V, S612R, |
| | 2494ΔG, E742K and M747K. |
| SEQ ID NO: 37 | SEQ ID NO: 1 incorporating the amino acid alterations P10S, |
| | A61V, F73S, T186I, R205K, K219E, M236T, A608V, S612R, |
| | 2494ΔG, E742R, and M747R. |
| SEQ ID NO: 38 | SEQ ID NO: 1 incorporating the amino acid alterations P10S, |
| | A61V, F73S, T186I, R205K, K219E, M236T, A608V, S612R, |
| | 2494ΔG, and E742N, and M747N. |
| SEQ ID NO: 39 | SEQ ID NO: 1 incorporating the amino acid alterations P10S, |
| | A61V, F73S, T186I, R205K, K219E, M236T, A608V, S612R, |
| | 2494ΔG, E742K, and M747R. |
| SEQ ID NO: 40 | SEQ ID NO: 1 incorporating the amino acid alterations P10S, |
| | A61V, F73S, T186I, R205K, K219E, M236T, A608V, S612R, |
| | 2494ΔG, E742K, and M747N. |
| SEQ ID NO: 41 | SEQ ID NO: 1 incorporating the amino acid alterations P10S, |
| | A61V, F73S, T186I, R205K, K219E, M236T, A608V, S612R, |
| | 2494ΔG, E742R, and M747K. |
| SEQ ID NO: 42 | SEQ ID NO: 1 incorporating the amino acid alterations P10S, |
| | A61V, F73S, T186I, R205K, K219E, M236T, A608V, S612R, |
| | 2494ΔG, E742R, and M747N. |
| SEQ ID NO: 43 | SEQ ID NO: 1 incorporating the amino acid alterations P10S, |
| | A61V, F73S, T186I, R205K, K219E, M236T, A608V, S612R, |
| | 2494ΔG, E742N, and M747K. |
| SEQ ID NO: 45 | SEQ ID NO: 1 incorporating the amino acid alterations P10S, |
| | A61V, F73S, T186I, R205K, K219E, M236T, A608V, S612R, |
| | 2494ΔG, E742N and E747R. |
| SEQ ID NO: 46 | SEQ ID NO: 1 incorporating the amino acid alterations P10S, |
| | L30P, A61V, L365P, V586A, S612R, E832K, and D732. |
| SEQ ID NO: 47 | SEQ ID NO: 1 incorporating the amino acid alterations P10S, |
| | L30P, A61V, L365P, V586A, S612R, E832K, E742K, and M747K. |
| SEQ ID NO: 48 | SEQ ID NO: 1 incorporating the amino acid alterations P10S, |
| | L30P, A61V, L365P, V586A, S612R, E832K, E742R, and M747R. |
| SEQ ID NO: 49 | SEQ ID NO: 1 incorporating the amino acid alterations P10S, |
| | L30P, A61V, L365P, V586A, S612R, E832K, E742N, and M747N. |
| SEQ ID NO: 50 | SEQ ID NO: 1 incorporating the amino acid alterations P10S, |
| | L30P, A61V, L365P, V586A, S612R, E832K, E742K, and M747R. |
| SEQ ID NO: 51 | SEQ ID NO: 1 incorporating the amino acid alterations P10S, |
| | L30P, A61V, L365P, V586A, S612R, E832K, E742K, and M747N. |
| SEQ ID NO: 52 | SEQ ID NO: 1 incorporating the amino acid alterations P10S, |
| | L30P, A61V, L365P, V586A, S612R, E832K, E742R, and M747K. |
| SEQ ID NO: 53 | SEQ ID NO: 1 incorporating the amino acid alterations P10S, |
| | L30P, A61V, L365P, V586A, S612R, E832K, E742R, and M747N. |
| SEQ ID NO: 54 | SEQ ID NO: 1 incorporating the amino acid alterations P10S, |
| | L30P, A61V, L365P, V586A, S612R, E832K, E742N, and M747K. |
| SEQ ID NO: 55 | SEQ ID NO: 1 incorporating the amino acid alterations P10S, |
| | L30P, A61V, L365P, V586A, S612R, E832K, E742N, and E747R. |
| SEQ ID NO: 56 | SEQ ID NO: 1 incorporating the amino acid alterations P10S, |
| | A61V, D244V, S612R, E832K, and D732N. |
| SEQ ID NO: 57 | SEQ ID NO: 1 incorporating the amino acid alterations P10S, |
| | A61V, D244V, S612R, E832K, E742K, and M747K. |
| SEQ ID NO: 58 | SEQ ID NO: 1 incorporating the amino acid alterations P10S, |
| | A61V, D244V, S612R, E832K, M742R, and M747R. |
| SEQ ID NO: 59 | SEQ ID NO: 1 incorporating the amino acid alterations P10S, |
| | A61V, D244V, S612R, E832K, E742N, and M747N. |
| SEQ ID NO: 60 | SEQ ID NO: 1 incorporating the amino acid alterations P10S, |
| | A61V, D244V, S612R, E832K, E742K, and M747R. |
| SEQ ID NO: 61 | SEQ ID NO: 1 incorporating the amino acid alterations P10S, |
| | A61V, D244V, S612R, E832K, E742K, and M747N. |
| SEQ ID NO: 62 | SEQ ID NO: 1 incorporating the amino acid alterations P10S, |
| | A61V, D244V, S612R, E832K, E742R, and M747K. |
| SEQ ID NO: 63 | SEQ ID NO: 1 incorporating the amino acid alterations P10S, |
| | A61V, D244V, S612R, E832K, E742R, and M747N. |
| SEQ ID NO: 64 | SEQ ID NO: 1 incorporating the amino acid alterations P10S, |
| | A61V, D244V, S612R, E832K, E742N, and M747K. |
| SEQ ID NO: 65 | SEQ ID NO: 1 incorporating the amino acid alterations P10S, |
| | A61V, D244V, S612R, E832K, E742N, and E747R. |
| SEQ ID NO: 66 | SEQ ID NO: 1 incorporating the amino acid alterations L30P, |
| | 2494ΔG, and D732N. |
| SEQ ID NO: 67 | SEQ ID NO: 1 incorporating the amino acid alterations L30P, |
| | 2494ΔG, E742K, and M747K. |
| SEQ ID NO: 68 | SEQ ID NO: 1 incorporating the amino acid alterations L30P, |
| | 2494ΔG, E742R, and M747R. |
| SEQ ID NO: 69 | SEQ ID NO: 1 incorporating the amino acid alterations L30P, |
| | 2494ΔG, E742N, and M747N. |
| SEQ ID NO: 70 | SEQ ID NO: 1 incorporating the amino acid alterations L30P, |
| | 2494ΔG, E742K, and M747R. |
| SEQ ID NO: 71 | SEQ ID NO: 1 incorporating the amino acid alterations L30P, |
| | 2494ΔG, E742K, and M747N. |
| SEQ ID NO: 72 | SEQ ID NO: 1 incorporating the amino acid alterations L30P, |
| | 2494ΔG, E742R, and M747K. |
| SEQ ID NO: 73 | SEQ ID NO: 1 incorporating the amino acid alterations L30P, |
| | 2494ΔG, E742R and M747N. |
| SEQ ID NO: 74 | SEQ ID NO: 1 incorporating the amino acid alterations L30P, |
| | 2494ΔG, E742N, and M747K. |
| SEQ ID NO: 75 | SEQ ID NO: 1 incorporating the amino acid alterations L30P, |
| | 2494ΔG, E742N, and E747R. |
| SEQ ID NO: 76 | SEQ ID NO: 1 incorporating the amino acid alterations A29T, |
| | G200S, D237G, F749I, and D732N. |
| SEQ ID NO: 77 | SEQ ID NO: 1 incorporating the amino acid alterations A29T, |
| | G200S, D237G, F749I, E742K, and M747K. |
| SEQ ID NO: 78 | SEQ ID NO: 1 incorporating the amino acid alterations A29T, |
| | G200S, D237G, F749I, E742R, and M747R. |
| SEQ ID NO: 79 | SEQ ID NO: 1 incorporating the amino acid alterations A29T, |
| | G200S, D237G, F749I, E742N, and M747N. |
| SEQ ID NO: 80 | SEQ ID NO: 1 incorporating the amino acid alterations A29T, |
| | G200S, D237G, F749I, E742K, and M747R. |
| SEQ ID NO: 81 | SEQ ID NO: 1 incorporating the amino acid alterations A29T, |
| | G200S, D237G, F749I, E742K, and M747N. |
| SEQ ID NO: 82 | SEQ ID NO: 1 incorporating the amino acid alterations A29T, |
| | G200S, D237G, F749I, E742R, and M747K. |
| SEQ ID NO: 83 | SEQ ID NO: 1 incorporating the amino acid alterations A29T, |
| | G200S, D237G, F749I, E742R, and M747N. |
| SEQ ID NO: 84 | SEQ ID NO: 1 incorporating the amino acid alterations A29T, |
| | G200S, D237G, F749I, E742N, and M747K. |
| SEQ ID NO: 85 | SEQ ID NO: 1 incorporating the amino acid alterations A29T, |
| | G200S, D237G, F749I, E742N, and E747R. |

1.-134. (canceled)

135. A composition for performing a reverse transcriptase reaction comprising:
a thermostable reverse transcriptase or a fragment thereof;
a reverse transcriptase (RT) buffer;
one or more template RNAs and deoxyribonucleoside triphosphates (dNTPs);
one or more low molecular weight polar organic solvents; and
optionally a DNA-dependent DNA polymerase enzyme or a fragment thereof.

136. The composition of claim 135, wherein the one or more low molecular weight polar organic solvents are selected from the group consisting of an amide, a sulfoxide, a sulfone, and a diol, the one or more low molecular weight organic solvents being present in the PCR buffer at a concentration ranging between about 0.05 molar and 7.5 molar.

137. The composition of claim 135, wherein molecular weight of the polar organic cosolvent is less than or equal to 150 g/mol.

138. The composition of claim 137, wherein the one or more polar organic solvents display a rate of change of duplex DNA, DNA secondary structure or RNA secondary structure melting temperature with respect to cosolvent concentration (dTm/d[solvent]) between −1 K/M and −15 K/M.

139. The composition of claim 138, wherein the duplex DNA corresponds to the c-jun DNA segment flanked by primers with SEQ IDs 92 and 94, and where the DNA and RNA secondary structures correspond to the most stable secondary structures in the single-stranded BEGAIN DNA and RNA fragments flanked by primers with SEQ IDs 126 and 127, respectively.

140. The composition of claim 135, wherein the thermostable reverse transcriptase enzyme has an optimal RT temperature above 37° C. and preferably above 48° C.

141. The composition of claim 135, wherein presence of the one or more organic cosolvents increases RT activity (rate of nucleotide incorporation) of the thermostable reverse transcriptase or the fragment thereof.

142. The composition of claim 141, wherein the RT activity rate is increased by at least 5%.

143. The composition of claim 135, wherein the one or more low molecular weight organic solvents is of the formula: 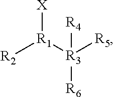
wherein:
R₁ is C or S; and
when R₁ is C, X is ═O, R₃ is N and R₆ is absent;
when R₁ is S, X is ═O or [symbol]
and R₃ is C;
R₂ is H or CH₃ only when one or more of R₄, R₅ and R₆ is not H, and otherwise
R₂ is an unsubstituted or halogen-, hydroxy- or alkoxy-substituted alkyl or cycloalkyl of length m, wherein m is selected such that the total number of carbons in the compound is between 3 and 8 when R₁ is C and between 2 and 8 when R₁ is S; wherein any two of R₂, R₃, R₄, R₅ and R₆ optionally form a cyclic structure in which cyclization is effected through a bond between them; and R₄, R₅ and R₆ each is H, alkyl, cycloalkyl or halogen-, hydroxy- or alkoxy-substituted alkyl or cycloalkyl of length n, wherein n is selected such that the total number of carbons in the compound is between 3 and 8 when R₁ is C and between 2 and 8 when R₁ is S and when R₄ and R₅ are CH₃, R₂ cannot be H or CH₃.

144. The composition of claim 143, wherein the one or more polar organic solvents comprises a cyclic compound, wherein the cyclization is effected through a bond between any two of R₂, R₃, R₄, R₅ and R₆.

145. The composition of claim 143, wherein the one or more low molecular weight polar organic solvents comprises a compound in which R₁ is S, X is ═O or [symbol]
, and R₃ is C.

146. The composition of claim 145, wherein the compound is cyclic.

147. The composition of claim 146, wherein the compound is selected from the group consisting of tetramethylene sulfone and tetramethylene sulfoxide.

148. The composition of claim 145, wherein the compound is acyclic.

149. The composition of claim 135 where the thermostable reverse transcriptase is a mutant of a thermostable DNA polymerase bearing at least 90% sequence similarity to the wild-type DNA polymerase or to a fragment thereof.

150. The composition of claim 149, wherein the thermostable reverse transcriptase comprises one or more non-natural amino acid alterations conferring stability and/or activity in the one or more polar organic cosolvents.

151. The composition of claim 150, wherein one or more non-natural amino acid alterations confers an increase in half-life of the thermostable reverse transcriptase of at least 50 percent at 95° C.

152. The composition of claim 150, wherein one or more non-natural amino acid alterations confers an increase in reverse transcriptase activity of at least 50% at 72° C.

153. The composition of claim 149, wherein the one or more amino acid alterations are selected from the group consisting of L5Q, F8L, P10S, L16P, A23P, A29T, K31R, G38D, A61V, P89S, A97T, A118V, L162P, K171T, T186I, E201K, R205K, K206Q, G208S, K219E, N220D, I228V, M236T, D244E, D244V, R261H, D273G, L287Q, S290G, V310L, H333R, K346R, L351M, P382T, E388D, E434D, A454E, L461Q, L461R, V474I, F482I, I503T, E507K, S515N, A521V, Q534R, S543G, D551G, D551N, Q592R, L606M, A608V, S612R, H676L, Q680R, K702R, D732N, E734G, S739G, E742K, F749I, F749V, F749L, K762R, K767R, L768M, Q782H, and E832K of SEQ ID NO:1.

154. The composition of claim 135, wherein the DNA-dependent DNA polymerase enzyme is present and is a solvostable DNA polymerase which is active, and stable at greater than 95° C., at at least one concentration between 1.0 molar to 3.0 molar of the one or more polar organic solvents.

155. The composition of claim 154, wherein the thermostable reverse transcriptase and DNA-dependent DNA polymerase enzyme are the same enzyme, and the RT buffer comprises a reverse transcriptase polymerase chain reaction (RT-PCR) buffer.

156. The composition of claim 154, wherein the solvostable DNA-dependent DNA polymerase is a mutant of a thermostable DNA polymerase bearing at least 90% sequence similarity to wild-type.

157. The composition of claim 156, wherein the mutant DNA polymerase comprises one or more amino acid alterations enhancing stability of the mutant polymerase in the one or more low molecular weight polar organic solvents.

158. The composition of claim 135 wherein the organic solvent is selected from the group consisting of 1,3-propanediol at a concentration of about 0.5 molar to about the concentration at which the melting temperature (T_{M}) of the reverse transcriptase is 5° C. higher than the optimal activity temperature of the reverse transcriptase in the absence of 1,3-propanediol; 1,4-butanediol at a concentration of about 0.5 molar to about the concentration at which the melting temperature (T_{M}) of the reverse transcriptase is 5° C. higher than the optimal activity temperature of the reverse transcriptase in the absence of 1,4-butanediol; 1,5-pentanediol at a concentration of about 0.5 molar to about the concentration at which the melting temperature (T_{M}) of the reverse transcriptase is 5° C. higher than the optimal activity temperature of the reverse transcriptase in the absence of 1,5-pentanediol; N,N-Dimethylformamide (DMF) at a concentration of about 0.5 molar to about the concentration at which the melting temperature (T_{M}) of the reverse transcriptase is 5° C. higher than the optimal activity temperature of the reverse transcriptase in the absence of DMF; isobutyramide at a concentration of about 0.1 molar to about the concentration at which the melting temperature (T_{M}) of the reverse transcriptase is 5° C. higher than the optimal activity temperature of the reverse transcriptase in the absence of isobutyramide; 2-pyrrolidone at a concentration of about 0.1 molar to about the concentration at which the melting temperature (T_{M}) of the reverse transcriptase is 5° C. higher than the optimal activity temperature of the reverse transcriptase in the absence of 2-pyrrolidone; N-methylpyrrolidone at a concentration of about 0.1 molar to about the concentration at which the melting temperature (T_{M}) of the reverse transcriptase is 5° C. higher than the optimal activity temperature of the reverse transcriptase in the absence of N-methylpyrrolidone; dimethylsulfoxide (DMSO) at a concentration range of about 0.5 molar to about the concentration at which the melting temperature (T_{M}) of the reverse transcriptase is 5° C. higher than the optimal activity temperature of the reverse transcriptase in the absence of DMSO; tetramethylene sulfoxide at a concentration range of about 0.1 molar to about the concentration at which the melting temperature (T_{M}) of the reverse transcriptase is 5° C. higher than the optimal activity temperature of the reverse transcriptase in the absence of tetramethylene sulfoxide; or tetramethylenesulfone (sulfolane) at a concentration of about 0.1 molar to about the concentration at which the melting temperature (T_{M}) of the reverse transcriptase is 5° C. higher than the optimal activity temperature of the reverse transcriptase in the absence of sulfolane.

159. The composition of claim 135, wherein the reverse transcriptase enzyme or a fragment thereof is a solvostable DNA polymerase which is active, and stable at greater than 90° C., in the presence of at least one concentration between 1.0 molar to 3.0 molar of the one or more polar organic solvents.

160. The composition of claim 159, wherein the solvostable DNA polymerase is a mutant of Taq polymerase bearing at least 90% sequence similarity to wild-type Taq polymerase of SEQ ID NO:1 or to the Stoffel fragment of Taq polymerase.

161. The composition of claim 156, wherein the mutant DNA polymerase includes one or more amino acid alterations conferring reverse transcriptase activity.

162. The composition of claim 135 wherein the reverse transcriptase is a modified DNA polymerase having an amino acid sequence that is at least 90% identical to an amino acid sequence comprised of the sequence of wild-type DNA polymerase or fragment thereof, wherein the amino acid sequence comprises a first set of non-natural amino acid alterations stabilizing and rendering the modified DNA polymerase more active in an aqueous-organic medium, and a second set of non-natural amino acid alterations conferring confer reverse transcriptase activity to the modified DNA polymerase.

163. The modified DNA polymerase of claim 162, wherein the first set of non-natural amino acid alterations are selected from the group consisting of L5Q, F8L, P10S, L16P, A23P, A29T, K31R, G38D, A61V, P89S, A97T, A118V, L162P, K171T, T186I, E201K, R205K, K206Q, G208S, K219E, N220D, I228V, M236T, D244E, D244V, R261H, D273G, L287Q, S290G, V310L, H333R, K346R, L351M, P382T, E388D, E434D, A454E, L461Q, L461R, V474I, F482I, I503T, E507K, S515N, A521V, Q534R, S543G, D551G, D551N, Q592R, L606M, A608V, S612R, H676L, Q680R, K702R, D732N, E734G, S739G, F749I, F749V, F749L, K762R, K767R, L768M, Q782H, and E832K of SEQ ID NO: 1,
and wherein the second set of non-natural amino acid alterations are selected from the group consisting of D732N, E742K, E742R, M747K, M747R, E742N, E742Q, E742Y, E742M, E742A and M747N of SED ID NO: 1.

164. The composition of claim 136, wherein the amide is selected from the group consisting of formamide, N-methyl formamide, N,N-dimethyl formamide (DMF), acetamide, N-methylacetamide, N,N-dimethylacetamide, propionamide, isobutyramide, 2-pyrrolidone, N-methylpyrrolidone (NMP), N-hydroxyethyl pyrrolidone(HEP), N-formyl pyrrolidine, and N-Formyl morpholine, wherein the sulfoxide is selected from the group consisting of dimethyl sulfoxide (DMSO), n-propyl sulfoxide, n-butyl sulfoxide, methyl sec-butyl sulfoxide, and tetramethylene sulfoxide; the sulfone is selected from the group consisting of dimethyl sulfone, diethylsulfone, di(n-isopropyl) sulfone, tetramethylene sulfone (sulfolane), 2,4-dimethylsulfolane, and butadienesulfone (sulfolene), and wherein the diol is selected from the group consisting of 1,2-propanediol, 1,3-propanediol, 1,2-butanediol, 1,3-butanediol, 1,4-butanediol, 1,2-pentanediol, 2,4-pentanediol, 1,5-pentanediol, 1,2-cyclopentanediol, 1,2-hexanediol, 1,6-hexanediol, and 2-methyl-2,4-pentanediol.

165. A reverse transcription kit or reverse transcription-PCR kit comprising the composition of claim 135, wherein the kit a) prescribes use of reverse transcription temperatures above 48° C. and below the melting temperature of the RNA:DNA heteroduplex in the presence of the employed concentration of the cosolvent, or below the melting temperature of the reverse transcriptase protein in the presence of the employed concentration of the cosolvent; or b) prescribes preincubation at a temperature more than 5° C. below the melting temperature of the primer:template complex calculated in the absence of cosolvent.

166. A method of reverse transcribing RNA into DNA employing the composition of claim 135.

167. A method of next-generation sequencing of RNA, wherein reverse transcription of RNA and/or DNA amplification is administered according to the method as in claim 166, and wherein RNA sequencing is carried out with or without the incorporation of unique molecular identifiers (UMI) in the adapter sequences.

168. The method of claim 167, wherein accuracy with which the copy numbers of sequences are determined from RNA sequencing is improved due to the inclusion of the polar organic cosolvent.
